(19) United States
(12) Patent Application Publication
(10) Pub. No.: US 2024/0199688 A1
(43) Pub. Date: Jun. 20, 2024

(54) METHODS FOR REDUCING LIPASE ACTIVITY

(71) Applicant: Regeneron Pharmaceuticals, Inc., Tarrytown, NY (US)

(72) Inventors: Xiaolin Tang, Tarrytown, NY (US); Leonid Breydo, Bronx, NY (US); John Mattila, Nyack, NY (US)

(21) Appl. No.: 18/310,921

(22) Filed: May 2, 2023

Related U.S. Application Data
(60) Provisional application No. 63/337,532, filed on May 2, 2022.
(60) Provisional application No. 63/436,850, filed on Jan. 3, 2023.
(60) Provisional application No. 63/499,441, filed on May 1, 2023.

(51) Int. Cl. C07K 1/16; A61K 47/26; C07K 1/36

(52) U.S. Cl. CPC C07K 1/16; A61K 47/26; C07K 1/36

(57) ABSTRACT

Pharmaceutical formulations comprising an antibody that specifically binds to human interleukin-4 receptor alpha (hIL-4Rα) are provided. The formulations may contain, in addition to an anti-IL-4Rα antibody, one or more buffers, at least one amino acid, at least one sugar, and a surfactant comprising a polysorbate, polyethylene glycol or a poloxamer. Methods for producing pharmaceutical formulations with reduced lipase activity are also provided, which may include subjecting a drug substance to anion exchange chromatography in acidic conditions, agitation stress, heat stress, and additional ion exchange or size exclusion chromatography. In one aspect, the pharmaceutical formulations do not have appreciable subvisible particle formation in the presence of lipase, and exhibit a substantial degree of antibody stability during storage and after being subjected to thermal and other physical stresses.

### CROSS-REFERENCE TO RELATED APPLICATIONS

This application incorporates by reference priority to and the benefit of Provisional Patent Application No. U.S. 63/337,532, filed on May 2, 2022, Provisional Patent Application No. 63/436,850 filed on Jan. 3, 2023, and Provisional Patent Application No. 63/499,441 filed on May 1, 2023.

### SEQUENCE LISTING

The instant application contains a Sequence Listing which has been filed electronically in xml format and is hereby incorporated by reference in its entirety. Said .xml copy, created on Jan. 31, 2023, is named 070816-03480.xml and is 180,926 bytes in size.

### FIELD

The present invention relates to the field of therapeutic protein formulations. More specifically, the present invention relates to the field of pharmaceutical formulations with reduced esterase or lipase activity and/or reduced formation of free fatty acid particles.

### BACKGROUND

Among drug products, protein-based biotherapeutics are an important class of drugs that offer a high level of selectivity, potency and efficacy, as evidenced by the considerable increase in clinical trials with monoclonal antibodies (mAbs) over the past several years. One critical aspect for a clinically and commercially viable biotherapeutic is stability of the drug product in terms of the manufacturing process, as well as shelf-life. Surfactants, such as polysorbate, are often used to enhance the physical stability of a protein-based biotherapeutics product. Over seventy percent of marketed monoclonal antibody therapeutics contain between 0.001% and 0.2% polysorbate, a type of surfactant, to impart physical stability to the protein-based biotherapeutics.

Enzymatic hydrolysis of polysorbate has been recognized as the primary route of polysorbate degradation in biotherapeutics formulations. Polysorbate degradation may be caused by co-purification of esterases or lipases during production of a drug product. Polysorbate hydrolysis results in the release of free fatty acids that can drive undesirable particulate formation in a drug product. Particles may be visible or subvisible; subvisible particles typically are under 150 microns or 100 microns in diameter.

Thus, a need exists for drug products with reduced esterase or lipase activity and reduced formation of free fatty acid particles, and for methods for making the same.

### SUMMARY

Products and methods have been developed for reducing esterase activity in a composition or formulation, reducing lipase activity in a composition or formulation, reducing polysorbate degradation, and reducing fatty acid particle formation. In one aspect, a sample comprising a protein of interest and an esterase or lipase is subjected to hydrophobic interaction chromatography to produce a formulated drug substance and/or drug product with reduced esterase and/or lipase activity, reduced polysorbate degradation, and/or reduced free fatty acid particle formation compared to a sample that was not subjected to hydrophobic interaction chromatography. In one aspect, a sample comprising a protein of interest and an esterase or lipase is subjected to anion exchange chromatography using a high load pH to produce a formulated drug substance and/or drug product with reduced esterase and/or lipase activity, reduced polysorbate degradation, and/or reduced fatty acid particle formation compared to a sample that was not subjected to anion exchange chromatography with a high load pH.

In one aspect, a sample comprising a protein of interest and an esterase or lipase is formulated with a polysorbate comprising a high concentration of oleic acid, for example≥ 98% oleic acid, to produce a formulated drug substance and/or drug product with reduced esterase and/or lipase activity, reduced polysorbate degradation, and/or reduced fatty acid particle formation compared to a sample that was not formulated with the polysorbate. Alternatively, in one aspect, a sample comprising a protein of interest and an esterase or lipase is formulated with poloxamer 188 or PEG3350 to produce a formulated drug substance and/or drug product with reduced esterase and/or lipase activity, reduced polysorbate degradation, and/or reduced fatty acid particle formation compared to a sample that was not formulated with poloxamer 188 or PEG3350. In some aspects, the protein of interest is an anti-IL4Rα antibody. In some aspects, the anti-IL-4Rα antibody is Dupilumab.

In one aspect, a formulated drug substance comprising a protein of interest, a fatty acid ester and an esterase or lipase is subjected to stress, for example agitation stress or heat stress, to inactivate the esterase or lipase, degrade the esterase or lipase, and/or reduce esterase or lipase activity, which may cause the formation of high molecular weight (HMW) species of esterase or lipase and/or drug protein. The substance may then be subjected to filtration and/or separation, for example using cation exchange chromatography or size exclusion chromatography, to remove HMW species and produce a formulated drug substance and/or drug product with reduced esterase or lipase activity, reduced polysorbate degradation, and/or reduced free fatty acid particle formation compared to a sample that was not subjected to stress and/or filtration/separation. In some aspects, the protein of interest is an anti-IL4Rα antibody. In some aspects, the anti-IL-4Rα antibody is Dupilumab.

This disclosure provides additional methods for producing a pharmaceutical composition with reduced esterase or lipase. In some aspects, these methods can comprise subjecting a sample including a protein of interest and an esterase or lipase to anion exchange chromatography, wherein a pH of the sample loaded to the chromatography column is between about 7.8 and about 8.3.

In some additional aspects, methods for producing a pharmaceutical composition with reduced esterase or lipase activity can comprise (a) subjecting a sample including a protein of interest and an esterase or lipase to stress conditions to form a sample with inactivated esterase or lipase; and (b) formulating said sample with inactivated esterase or lipase to produce a pharmaceutical composition with reduced esterase or lipase activity.

In one aspect, said protein of interest is an antibody, an antibody-derived protein, an antibody fragment, a monoclonal antibody, a bispecific antibody, a fusion protein, an antibody-drug conjugate, or a therapeutic protein.

In one aspect, said formulating step comprises adding a fatty acid ester to said sample. In a specific aspect, said fatty acid ester is polysorbate 20, polysorbate 40, polysorbate 60, or polysorbate 80. In a specific aspect, said fatty acid ester is polysorbate 80 and a concentration of oleic acid esters in said polysorbate 80 is at least 80%. In more specific aspect, a concentration of oleic acid esters in said polysorbate 80 is at least 98% or at least 99%.

In one aspect, said stress conditions include agitation stress and/or heat stress. In a specific aspect, said agitation stress comprises shaking said sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm. In another specific aspect, said agitation stress comprises shaking said sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.

In an additional specific aspect, said heat stress comprises storing said sample at from about 20° ° C. to about 60° C., from about 25° C. to about 55° C., from about 40° C. to about 50° C., from about 44° C. to about 46° C., about 20° C., about 25° C., about 30° C., about 35° C., about 40° C., about 45° ° C., about 50° C., about 55° C., or about 60° C. In a more specific aspect, said storage is from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.

In one aspect, the method further comprises subjecting said sample with inactivated esterase or lipase to filtration, enrichment, or chromatographic separation to remove inactivated lipase and/or protein HMW species prior to step (b). In a specific aspect, said chromatographic separation comprises cation exchange chromatography. In another specific aspect, said chromatographic separation comprises size exclusion chromatography.

In one aspect, said formulating step comprises adding excipients to said sample.

This disclosure provides further methods for producing a pharmaceutical composition with reduced esterase or lipase activity. In some aspects, the methods can comprise (a) subjecting harvested antibody to affinity chromatography; (b) subjecting said antibody pooled from eluate of step (a) to viral inactivation at a pH from about 3 to about 4 and then adjusting the pH to from about 5 to about 8; (c) subjecting said antibody pooled from step (b) to anion exchange chromatography in flowthrough mode; (d) subjecting said antibody pooled from eluate of step (c) to hydrophobic interaction chromatography in flowthrough mode; (e) subjecting said antibody pooled from flowthrough fractions of step (d) to virus retentive filtration; (f) subjecting a sample from step (e) including an antibody of interest and an esterase or lipase to agitation stress or heat stress to form a pharmaceutical composition with reduced esterase or lipase activity.

In other aspects, the methods can comprise (a) subjecting harvested antibody to affinity chromatography; (b) subjecting said antibody pooled from eluate of step (a) to viral inactivation at a pH from about 3 to about 4 and then adjusting the pH to from about 5 to about 8; (c) subjecting said antibody pooled from step (b) to anion exchange chromatography in flowthrough mode; (d) subjecting said antibody pooled from flowthrough fractions of step (c) to virus retentive filtration; (e) subjecting a sample from step (d) including an antibody of interest and an esterase or lipase to agitation stress or heat stress to form a pharmaceutical composition with reduced esterase or lipase activity.

In one aspect, the methods further comprise subjecting the pharmaceutical composition to filtration, enrichment, or chromatographic separation to remove HMW species. In a specific aspect, the chromatographic separation comprises ion exchange chromatography, cation exchange chromatography, or size exclusion chromatography.

In one aspect, a pH of the sample loaded to the AEX column is between about 7.8 and about 8.3.

This disclosure provides methods for producing a pharmaceutical composition with increased stability. In some aspects, the methods can comprise reducing esterase or lipase activity in a composition by subjecting a sample including a protein of interest, an esterase or lipase, and a fatty acid ester to anion exchange (AEX) chromatography, wherein a pH of the sample loaded to the AEX column is between about 7.8 and about 8.3.

In one aspect, the method further comprises the step of subjecting a sample formed after AEX chromatography to heat stress or agitation stress to reduce esterase or lipase activity. In a specific aspect, the method further comprises subjecting said stressed sample to chromatographic separation to remove HMW species of said esterase or lipase.

In one aspect, the method further comprises the addition of poloxamer 188 or PEG3350 to produce a formulation comprising a pharmaceutical composition with reduced esterase or lipase activity and increased stability. In a specific aspect, the formulation is substantially free of polysorbate. In another specific aspect, a concentration of said poloxamer 188 is from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%. In yet another specific aspect, a concentration of said PEG3350 is from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%.

This disclosure provides additional methods for producing a pharmaceutical composition with increased stability. In some aspects, the methods can comprise (a) subjecting a harvested recombinant protein to anion exchange chromatography in flowthrough mode; (b) subjecting flowthrough fractions from step (a) to hydrophobic interaction chromatography in flowthrough mode; and (c) formulating recombinant protein isolated from step (b) with polysorbate 80, wherein a concentration of oleic acid esters in said polysorbate 80 is at least 80%.

In one aspect, a concentration of said oleic acid esters at least 98% or at least 99%.

In one aspect, a pH of the sample loaded to the anion exchange chromatography column is from about 7.8 to about 8.3, from about 7.9 to about 8.2, about 7.8, about 7.9, about 8.0, about 8.1, about 8.2, or about 8.3.

In some aspects, the protein of interest or recombinant protein is an anti-IL4Rα antibody. In some aspects, the anti-IL-4Rα antibody is Dupilumab.

These, and other, aspects of the invention will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows a chromatogram showing relative amounts of different molecular species with (A) lower quality polysorbate 20 (PS20-A), (B) higher quality polysorbate 20 (PS20-B), and (C) polysorbate 80 (PS80), according to one aspect.

FIG. 2A shows the formation of particles in mAb1 formulations comprising SR-PS80 as measured by membrane microscopy, according to one aspect.

FIG. 2B shows the formation of particles in mAb1 formulations comprising HP-PS20 as measured by membrane microscopy, according to one aspect.

FIG. 2C shows the formation of particles in mAb1 formulations comprising SR-PS80 as measured by MFI, according to one aspect.

FIG. 2D shows the formation of particles in mAb1 formulations comprising HP-PS20 as measured by MFI, according to one aspect.

FIG. 3A shows recovery of polysorbate in mAb1 formulations comprising SR-PS80 as measured by CAD-UHPLC, according to one aspect.

FIG. 3B shows recovery of polysorbate in mAb1 formulations comprising HP-PS20 as measured by CAD-UHPLC, according to one aspect.

FIG. 4 shows a correlation between formation of particles and recovery of polysorbate in mAb1 formulations, according to one aspect.

FIG. 5 shows a correlation between formation of particles and recovery of polysorbate in mAb5 formulations, according to one aspect.

FIG. 6A shows the formation of particles in mAb1 formulations comprising SR-PS80 and subjected to HIC purification as measured by membrane microscopy, according to one aspect.

FIG. 6B shows the formation of particles in mAb1 formulations comprising HP-PS20 and subjected to HIC purification as measured by membrane microscopy, according to one aspect.

FIG. 6C shows the formation of particles in mAb1 formulations comprising SR-PS80 and subjected to HIC purification as measured by MFI, according to one aspect.

FIG. 6D shows the formation of particles in mAb1 formulations comprising HP-PS20 and subjected to HIC purification as measured by MFI, according to one aspect.

FIG. 7A shows recovery of polysorbate in mAb1 formulations comprising SR-PS80 and subjected to HIC purification as measured by CAD-UHPLC, according to one aspect.

FIG. 7B shows recovery of polysorbate in mAb1 formulations comprising HP-PS20 and subjected to HIC purification as measured by CAD-UHPLC, according to one aspect.

FIG. 8 shows a correlation between phospholipase activity (in parts per million) and percent degradation of polysorbate 20, according to one aspect.

FIG. 9 shows a visualization of models generated for a multivariate study of AEX risk factors and responses, according to one aspect.

FIG. 10 shows a comparison of AEX pool lipase activity (%) performance during confirmation batches compared to small-scale predictions from Monte Carlo simulations, according to one aspect.

FIG. 11 shows polysorbate recovery from formulations comprising mAb5, mAb6 and mAb7, according to one aspect.

FIG. 12 shows polysorbate recovery from mAb5 formulations at varying concentrations and incubation times, according to one aspect.

FIG. 13 shows homology modeling depicting surface hydrophobicity of mAb5, mAb6 and mAb7, according to one aspect.

FIG. 14 shows a number of subvisible particulates (≥ 10 μm) measured by the membrane miscroscopic method, in protein drug products comprising various types of polysorbate 80, according to one aspect.

FIG. 15 shows a number of subvisible particulates (≥ 10 μm) measured by micro-flow imaging (MFI), in protein drug products comprising various types of polysorbate 80, according to one aspect.

FIG. 16 shows a chemical structure of polyoxyethylene (20) sorbitan monooleate, the predominant fatty acid ester in polysorbate 80, according to one aspect.

FIG. 17 shows a measured concentration of free fatty acids in protein drug products comprising various types of polysorbate 80, according to one aspect.

FIG. 18A illustrates a polysorbate structure and main degradation routes, according to one aspect.

FIG. 18B illustrates a structure of poloxamer 188, according to one aspect.

FIG. 19 shows surfactant recovery for mAb5 formulations comprising PS20, PS80, or

poloxamer 188 (P188) at various temperatures, according to one aspect.

FIG. 20 shows a number of particles≥ 10 μm (upper panel) and ≥ 25 μm (lower panel) identified by membrane microscopy in a formulation of 150 mg/mL of an anti-IL-4R antibody containing a lipase and either PEG3350 or poloxamer 188 (at concentrations of 0.02%, 0.04%, or 0.1% w/v) and stored at 5° C. for up to 36 months, according to one aspect.

FIG. 21A shows an impact of PEG3350 concentrations on the stability (as a percentage of HMW species measured by SE-UPLC) of a formulation of 150 mg/mL of an anti-IL-4R antibody against agitation stress at room temperature for a period of from 30-120 minutes.

FIG. 21B shows an impact of poloxamer 188 concentrations on the stability (as a percentage of HMW species measured by SE-UPLC) of a formulation of 150 mg/mL of an anti-IL-4R antibody against agitation stress at room temperature for a period of from 30-120 minutes.

FIG. 22 shows an impact of polysorbate 20, polysorbate 80, PEG3350, and poloxamer 188 (at varying concentrations) on the stability (as a percentage of HMW species measured by SE-UPLC) of a formulation of 150 mg/mL of an anti-IL-4R antibody against thermal stress (45° C.)for a period of up to 56 days.

### DETAILED DESCRIPTION

Therapeutic macromolecules must be formulated in a manner that not only makes the molecules suitable for administration to patients, but also maintains their stability during storage. For example, therapeutic antibodies in liquid solution are prone to degradation, aggregation and/or undesired chemical modifications unless the solution is formulated properly. The stability of an antibody in liquid formulation depends not only on the kinds of excipients used in the formulation, but also on the amounts and proportions of the excipients relative to one another. Therapeutic formulations also may be subject to the formation of particulate matter over time during storage. Particles may be visible or subvisible; subvisible particles typically are under 150 microns or 100 microns in diameter. Formulations having high protein concentrations, e.g., concentrations of 30 mg/mL or higher, are more prone to aggregation and subvisible particle formation.

Polysorbate 20 (PS20) and polysorbate 80 (PS80) are the most commonly used nonionic surfactants in biopharmaceutical protein formulations for improving protein stability and protecting protein products from aggregation and denaturation (Martos et al., J Pharm Sci. 106(7):1722-1735 (2017); Kiese et al., J Pharm Sci 97(19):4347-4366 (2008); Dwivedi et al., Int J Pharm 552(1-2):422-436 (2018)). However, it has been reported that polysorbates, including polysorbate 20 and polysorbate 80, can degrade in the presence of lipase, which over time results in the formation of subvisible particles in a formulation.

PSs are known to be liable to degradation via two main pathways: autooxidation and hydrolysis (Dwivedi et al.; Kishore et al., Pharm Res. 28(5): 1194-1210 (2011); Larson et al., J Pharm Sci. 109(10):633-639 (2020); Kishore et al. J Pharm Sci. 100(2):721-731 (2011)). Enzymatic hydrolysis is considered to be the primary route of PS degradation in high-concentration protein formulations, which results in the accumulation of free fatty acids (FFAs) that may drive undesirable particulate formation in the drug products. Oxidation is the second primary pathway for PS degradation, which leads to the formation of peroxides, aldehydes, ketones and short-chain esterified POE sorbitan/isosorbide species (Kishore et al. 2011a; Larson et al.; Kishore et al. 2011b; Donbrow et al., J Pharm Sci. 67(12): 1676-1681 (1978); Yao et al. Pharm Res. 26(10):2303-2313 (2009)). PS hydrolysis has been recognized as a larger threat to drug product quality because this process not only reduces the PS concentration, but is also associated with particulate formation due to the low solubility of accumulated FFAs, especially at storage temperatures of 2° C.-8° C.(Doshi et al., J Pharm Sci. 110(2):687-692 (2021); Saggu et al., J Pharm Sci. 110(3):1093-1102 (2021); Doshi et al. Mol Pharm. 12(11):3792-3804 (2015)).

Residual lipases or esterase present in drug product are the major cause of PS hydrolysis (Chiu et al., Biotechnol Bioeng. 114(5): 1006-1015 (2017); Hall et al. J Pharm Sci. 105(5): 1633-1642 (2016); Labrenz et al. J Pharm Sci. 103(8):2268-2277 (2014); McShan et al. PDA J Pharm Sci Technol. 70(4):332-345 (2016); Zhang et al. J Pharm Sci. 109(11):3300-3307 (2020); Zhang et al. J Pharm Sci. 109(9):2710-2718 (2020)). The levels of residual lipases in final drug product are usually very low (<10 ppm) after multiple steps of downstream purification, and the consequences of PS degradation may not be noticeable until after storage for months or years at typical storage temperatures (2° C.-8° C.).

Without intending to be bound by theory, it is believed that a putative phospholipase B-like 2 (PLBL2), which is highly conserved in hamster, rat, mice, human and bovine, copurifies with some classes of proteins under certain processes. Other esterases or lipases may also copurify with proteins of interest at concentrations too low to be reliably detected but high enough to have measurable lipase activity, eventually resulting in a loss of polysorbate, production of free fatty acids, and the formation of visible or sub-visible particles. Therefore, a need exists for drug products with reduced lipase activity, reduced esterase activity, and reduced formation of fatty acid particles, and for methods for making the same.

Disclosed herein are novel drug products, compositions and formulations with reduced lipase activity, reduced esterase activity, and reduced formation of fatty acid particles, and methods for making the same. The inventors have surprisingly discovered that a hydrophobic interaction chromatography (HIC) step can be added to a production process for a protein of interest to effectively remove esterase and lipase activity and prevent the formation of particles in drug products. Furthermore, optimized conditions have been discovered for an anion exchange (AEX) chromatography step to effectively reduce esterase and lipase activity by improving the conventional pH load conditions. The inventors have additionally discovered that polysorbate content, specifically polysorbate 80, can be optimized with high oleic acid concentration, such that fewer fatty acid particles form even in drug product containing esterase or lipase activity. Alternative surfactants to polysorbate were also investigated, and it was surprisingly discovered that PEG3350 and poloxamer 188 can successfully stabilize a protein while being resistant to esterase or lipase activity, thereby preventing the formation of fatty acid particles. Following the production of a drug substance, it was further surprisingly discovered that co-purified esterases or lipases can be successfully inactivated and/or removed by the application of stress, for example agitation stress or heat stress, which can cause esterases or lipases to degrade, reducing lipase activity. The application of stress may also lead to the formation of high molecular weight (HMW) species. The HMW species can be removed from the drug substance using, for example, molecular weight filtration or chromatography, such as cation exchange chromatography or size exclusion chromatography. These, and other aspects of the invention are set forth in further detail below.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, bispecific antibodies, and antigen-binding proteins.

Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

### Antibodies

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In some aspects, the protein of interest is a human antibody. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Interleukin-4 (IL-4) and interleukin-13 (IL-13) are key cytokines in driving allergic and T helper cell type 2 (Th2) polarized inflammatory processes. IL-4 and IL-13 signaling is mediated through heterodimeric receptor complexes, in which IL-4 receptor alpha (IL-4Ra) is a shared receptor subunit for both IL-4 and IL-13 signaling. Thus, IL-4Rα is an attractive therapeutic target because it provides a single target for blocking both IL-4 and IL-13 signaling. In some aspects, the protein of interest is an anti-IL-4R antibody, or an antigen-binding fragment thereof. Antibodies to hIL-4Rα are described in, for example, U.S. Pat. Nos. 5,717,072, 7,186,809 and 7,605,237.

In some aspects, the anti-IL-4R antibody is a human IgG antibody. In various aspects, the anti-IL-4R antibody is a human antibody of isotype IgG1, IgG2, IgG3 or IgG4, or mixed isotype. In some aspects, the anti-IL-4R antibody is a human IgG1 antibody. In some aspects, the anti-IL-4R antibody is a human IgG4 antibody. In any of the aspects discussed above or herein, the anti-IL-4R antibody may comprise a human kappa light chain. In any of the aspects discussed above or herein, the anti-IL-4R antibody may comprise a human lambda light chain.

The antibodies of the disclosure may, in some aspects, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain aspects, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KA-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

An “isolated antibody,” as used herein, is intended to refer to an antibody that is substantially free of other antibodies having different antigenic specificities (e.g., an isolated antibody that specifically binds hIL-4Rα is substantially free of antibodies that specifically bind antigens other than hIL-4Ra).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4Rα molecules from other species (orthologs). In the context of the present disclosure, multispecific (e.g., bispecific) antibodies that bind to hIL-4Rα as well as one or more additional antigens are deemed to “specifically bind” hIL-4Ra. Moreover, an isolated antibody may be substantially free of other cellular material and/or chemicals. However, in some instances, the isolated antibody may be copurified with a phospholipase expressed by a mammalian cell line (e.g., CHO cells) from which the anti-IL-4R antibody is produced.

According to certain aspects of the present disclosure, the anti-hIL-4R antibody, or antigen-binding fragment thereof, comprises heavy chain complementarity determining regions HCDR1-HCDR2-HCDR3, respectively, comprising the amino acid sequences of SEQ ID NOs: 3-4-5. According to certain aspects of the present disclosure, the anti-hIL-4R antibody, or antigen-binding fragment thereof, comprises light chain complementarity determining regions LCDR1-LCDR2-LCDR3, respectively, comprising the amino acid sequences of SEQ ID NOs: 6-7-8. In certain aspects, the anti-hIL-4R antibody, or antigen-binding fragment thereof, comprises the CDRs HCDR1-HCDR2-HCDR3-LCDR1-LCDR2-LCDR3, respectively, comprising the amino acid sequences of SEQ ID NOs: 3-4-5-6-7-8.

In certain aspects, the anti-hIL-4R antibody, or antigen-binding fragment thereof, comprises heavy chain complementarity determining regions HCDR1-HCDR2-HCDR3, respectively, comprising the amino acid sequences of SEQ ID NOs: 3-4-5 and has a heavy chain variable region (HCVR) having at least 90% sequence identity (e.g., at least 91%, 92%, 93%, 94%, 95%, 96%, 97%, 98%, or 99% sequence identity) to the amino acid sequence of SEQ ID NO: 1. In certain aspects, the anti-hIL-4R antibody, or antigen-binding fragment thereof, comprises light chain complementarity determining regions LCDR1-LCDR2-LCDR3, respectively, comprising the amino acid sequences of SEQ ID NOs: 6-7-8 and has a light chain variable region (LCVR) having at least 90% sequence identity (e.g., at least 91%, 92%, 93%, 94%, 95%, 96%, 97%, 98%, or 99% sequence identity) to the amino acid sequence of SEQ ID NO: 2. In certain aspects, the anti-hIL-4R antibody, or antigen-binding fragment thereof, comprises: heavy chain complementarity determining regions HCDR1-HCDR2-HCDR3, respectively, comprising the amino acid sequences of SEQ ID NOs: 3-4-5 and has a heavy chain variable region (HCVR) having at least 90% sequence identity (e.g., at least 91%, 92%, 93%, 94%, 95%, 96%, 97%, 98%, or 99% sequence identity) to the amino acid sequence of SEQ ID NO: 1; and light chain complementarity determining regions LCDR1-LCDR2-LCDR3, respectively, comprising the amino acid sequences of SEQ ID NOs: 6-7-8 and has a light chain variable region (LCVR) having at least 90% sequence identity (e.g., at least 91%, 92%, 93%, 94%, 95%, 96%, 97%, 98%, or 99% sequence identity) to the amino acid sequence of SEQ ID NO: 2.

In certain aspects, the anti-hIL-4R antibody, or antigen-binding fragment thereof, comprises a heavy chain variable region (HCVR) comprising the amino acid sequence of SEQ ID NO: 1. In certain aspects, the anti-hIL-4R antibody, or antigen-binding fragment thereof, comprises a light chain variable region (LCVR) comprising the amino acid sequence of SEQ ID NO: 2. In certain aspects, the anti-hIL-4R antibody, or antigen-binding fragment thereof, comprises a HCVR/LCVR amino acid sequence pair comprising the amino acid sequences of SEQ ID NOs: ½.

In some aspects, the anti-IL-4R antibody comprises a HCVR/LCVR comprising the amino acid sequences of SEQ ID NOs: ½, respectively, and a human IgG1 heavy chain constant region. In some aspects, the anti-IL-4R antibody comprises a HCVR/LCVR comprising the amino acid sequences of SEQ ID NOs: ½, respectively, and a human IgG4 heavy chain constant region. In some aspects, the anti-IL-4R antibody comprises a HCVR/LCVR comprising the amino acid sequences of SEQ ID NOs: ½, respectively, and a human IgG heavy chain constant region. In some aspects, the anti-IL-4R antibody comprises a HCVR/LCVR comprising the amino acid sequences of SEQ ID NOs: ½, respectively, and a human IgG1 or IgG4 heavy chain constant region. In some aspects, the anti-IL-4R antibody comprises a heavy chain comprising the amino acid sequence of SEQ ID NO: 9 and a light chain comprising the amino acid sequence of SEQ ID NO: 10. In some aspects, the anti-IL-4R antibody is dupilumab.

Other anti-IL-4R antibodies that can be used in the context of the methods of the present disclosure include, for example, the antibody referred to and known in the art as AMG317 (Corren et al., 2010, Am J Respir Crit Care Med., 181(8): 788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141; US Patent Application Publication No. US2021/0238294; or International Patent Publication Nos. WO2019/228405, WO2020/096381, WO 2020/135471, WO2020/135710, or WO 2020/239134, the contents of each of which are incorporated by reference herein.

In some aspects, the anti-IL-4R antibody comprises one or more CDR, HCVR, and/or LCVR sequences set forth in Table 1 below.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain aspects, the pharmaceutical formulations may contain about 1 mg/mL to about 500 mg/mL of antibody; about 5 mg/mL to about 250 mg/ml of antibody; about 5 mg/mL to about 200 mg/mL of antibody; about 15 mg/mL to about 200 mg/ml of antibody; about 25 mg/mL to about 200 mg/mL of antibody; about 50 mg/mL to about 200 mg/mL of antibody; about 100 mg/mL to about 200 mg/mL of antibody; about 125 mg/mL to about 175 mg/mL of antibody; or about 150 mg/mL to about 200 mg/mL of antibody. For example, the formulations of the present disclosure may be liquid formulations that comprise about 1 mg/ml; about 2 mg/mL; about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof that binds specifically to hIL-4Ra. In certain aspects, the pharmaceutical formulations are liquid formulations that may contain 5+0.5 mg/mL to 200+20 mg/mL of antibody; 15+1.5 mg/mL to 200+20 mg/mL of antibody; 25+2.5 mg/mL to 200+20 mg/mL of antibody; 50+5 mg/mL to 200+20 mg/mL of antibody; 100+10 mg/mL to 200+20 mg/mL of antibody; 150+10 mg/mL of antibody; or 175+10 mg/mL. In some aspects, the pharmaceutical formulations contain from 140+5 mg/mL to 160+5 mg/mL of the anti-IL-4R antibody. In some cases, the pharmaceutical formulations contain 165 mg/mL+5 mg/mL to 185 mg/mL+5 mg/mL of the anti-IL-4R antibody. In some cases, the pharmaceutical formulations contain 150 mg/mL+5 mg/mL of the anti-IL-4R antibody. In some cases, the pharmaceutical formulations contain 175 mg/mL+5 mg/mL of the anti-IL-4R antibody.

The present disclosure encompasses antibodies having amino acid sequences that vary from those of the exemplary molecules disclosed herein but that retain the ability to bind the cognate antigen, for example hIL-4R. Such variant molecules may comprise one or more additions, deletions, or substitutions of amino acids when compared to the parent sequence, but exhibit biological activity that is essentially equivalent to that of the antibodies discussed herein.

The present disclosure includes antigen-binding molecules that are bioequivalent to any of the exemplary antibodies set forth herein. In some aspects, the antigen-binding molecule is a bioequivalent of dupilumab. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one aspect, two antibodies are bioequivalent if there are no clinically meaningful differences in their safety, purity, and potency. In one aspect, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods. Bioequivalence measures include, for example, (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) a well-controlled clinical trial that establishes safety, efficacy, or bioavailability or bioequivalence of an antigen-binding protein.

### Pharmaceutical Formulations

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product.

The terms “composition,” “formulation,” and “formulated drug substance” (FDS) as used in the present disclosure refer to a combination of two or more pharmaceutical ingredients for inclusion in a drug product. A composition, formulation, or FDS may be, for example, a liquid composition including an active pharmaceutical ingredient, such as an antibody, and an excipient, such as a stabilizer or surfactant. A composition, formulation, or FDS may include multiple excipients. A composition, formulation, or FDS may also include other constituents, such as host cell proteins co-purified with a protein of interest.

The term “drug product” (DP) as used in the present disclosure refers to a dosage form comprising an amount of a FDS for packaging, shipment, or administration. For example, a drug product may be a pre-filled syringe holding a volume of FDS for administration to a patient.

As used herein, a “protein pharmaceutical product,” “biopharmaceutical product,” “pharmaceutical formulation,” “pharmaceutical composition,” or “biotherapeutic” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

In some aspects, pharmaceutical formulations of the present invention comprise: (i) a human antibody that specifically binds to hIL-4Ra; (ii) one or more buffers; (iii) a thermal stabilizer; (iv) a surfactant (e.g., organic cosolvent); and (v) a viscosity modifier. Additional components may be included in the formulations of the present disclosure if such components do not significantly interfere with the viscosity and stability of the formulation. Specific exemplary components and formulations included within the present disclosure are described in detail below.

The pharmaceutical formulations of the present disclosure may, in certain aspects, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

### Host Cell Proteins

As used herein, the term “host cell protein” (HCP) includes protein derived from a host cell in the production of a recombinant protein. Host cell protein can be a process-related impurity which can be derived from the manufacturing process and can include three major categories: cell substrate-derived, cell culture-derived and downstream-derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

The presence of a host cell protein in a biotherapeutic product may be considered to be a higher or lower risk based on a number of measurable factors. One such factor is the concentration or abundance (quantity) of an HCP impurity in a biotherapeutic product. An HCP may have no discernible impact at a low enough abundance, as measured by, for example, ELISA or mass spectrometry. The level at which an HCP may present a considerable risk, which may be considered an unacceptable level in a product and may be monitored as a critical quality attribute (CQA), may depend on the specific identity of the HCP. Particular HCPs may be known to present a risk at a particular level, for example depending on the level of enzymatic activity of an HCP that is an enzyme.

Relatedly, the criticality of the presence of an HCP may depend on the function of that HCP, in particular in relation to the components of the biotherapeutic product. For example, an HCP esterase or lipase that may or is known to degrade polysorbate that is present in the biotherapeutic product of interest may be closely monitored and may have a low threshold for how much of the HCP impurity can be allowed in the biotherapeutic product. Other HCPs of particular concern may be, for example, proteases that may or are known to degrade a protein of interest in the biotherapeutic product, or immunogenic HCPs that may or are known to cause an immune reaction when administered to a subject.

### Liquid Chromatography

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection. In some aspects, a sample including at least one esterase or lipase, for example a drug substance, may be subjected to stress, for example agitation stress or heat stress, and subsequently subjected to a chromatography step to remove any HMW species.

In certain aspects, it may be advantageous to subject a biological sample to affinity chromatography for production of a protein of interest. The chromatographic material is capable of selectively or specifically binding to or interacting with the protein of interest. Non-limiting examples of such chromatographic material include Protein A and Protein G. Also included is chromatographic material comprising, for example, a protein or portion thereof capable of binding to or interacting with the protein of interest.

Affinity chromatography can involve subjecting a biological sample to a column comprising a suitable Protein A resin. When used herein, the term “Protein A” encompasses Protein A recovered from a native source thereof, Protein A produced synthetically (e.g., by peptide synthesis or by recombinant techniques), and variants thereof which retain the ability to bind proteins which have a C_{H}2/C_{H}3 region. In certain aspects, Protein A resin is useful for affinity-based production and isolation of a variety of antibody isotypes by interacting specifically with the Fc portion of a molecule should it possess that region.

There are several commercial sources for Protein A resin. Suitable resins include, but are not limited to, MabSelect PrismA, MabSelect SuRe™, MabSelect SuRe LX, MabSelect, MabSelect SuRe pcc, MabSelect Xtra, rProtein A Sepharose from Cytiva, ProSep HC, ProSep Ultra, ProSep Ultra Plus from EMD Millipore, MabCapture from ThermoFisher, and Amsphere™ A3 from JSR Life Sciences.

An affinity column can be equilibrated with a suitable buffer prior to sample loading. Following loading of the column, the column can be washed one or multiple times using a suitable wash buffer. The column can then be eluted using an appropriate elution buffer, for example, glycine-HCl, acetic acid, or citric acid. The eluate can be monitored using techniques well known to those skilled in the art such as a UV detector. The eluted fractions of interest can be collected and then prepared for further processing.

Cation exchange chromatography (CEX) uses a cation exchange chromatography material. Cation exchange chromatography can be further subdivided into, for example, strong cation exchange (SCX) or weak cation exchange, depending on the cation exchange chromatography material employed. Cation exchange chromatography materials with a sulfonic acid group (S) may be used in strong cation exchangers, while cation exchange chromatography materials with a carboxymethyl group (CM) may be used in weak cation exchangers. Strong cation exchangers include, for example SOURCE S, which uses a functional group of methyl sulfate, and SP Sepharose, which uses a functional group of sulfopropyl. Weak cation exchangers include, for example, CM-Cellulose, which uses a functional group of carboxymethyl. SCX may be preferred because a wider range of pH buffers may be used without losing the charge of the strong cation exchanger, allowing for effective separation of analytes with a wide pI range.

Cation exchange chromatography materials are available under different names from a multitude of companies such as, for example, Bio-Rex, Macro-Prep CM (available from BioRad Laboratories, Hercules, Calif., USA), weak cation exchanger WCX 2 (available from Ciphergen, Fremont, Calif., USA), Dowex MAC-3 (available from Dow chemical company, Midland, Mich., USA), Mustang C (available from Pall Corporation, East Hills, N.Y., USA), Cellulose CM-23, CM-32, CM-52, hyper-D, and partisphere (available from Whatman plc, Brentford, UK), Amberlite IRC 76, IRC 747, IRC 748, GT 73 (available from Tosoh Bioscience GmbH, Stuttgart, Germany), CM 1500, CM 3000 (available from BioChrom Labs, Terre Haute, Ind., USA), and CM-Sepharose Fast Flow (available from GE Healthcare, Life Sciences, Germany). In addition, commercially available cation exchange resins further include carboxymethyl-cellulose, Bakerbond ABX, sulphopropyl (SP) immobilized on agarose (e.g. SP-Sepharose Fast Flow or SP-Sepharose High Performance, available from GE Healthcare-Amersham Biosciences Europe GmbH, Freiburg, Germany) and sulphonyl immobilized on agarose (e.g. S-Sepharose Fast Flow available from GE Healthcare, Life Sciences, Germany).

Cation exchange chromatography materials include mixed-mode chromatography materials performing a combination of ion exchange and hydrophobic interaction technologies (e.g., Capto adhere, Capto MMC, MEP HyperCell, Eshmuno HCX, etc.), mixed-mode chromatography materials performing a combination of anion exchange and cation exchange technologies (e.g., hydroxyapatite, ceramic hydroxyapatite, etc.), and the like. In some aspects, CEX may be used to remove HMW species from a sample, for example to remove proteins that have aggregated into HMW species following agitation or heat stress.

In some aspects, a sample comprising a protein of interest is subjected to at least one anion exchange (AEX) separation step. Anion exchange packed bed chromatography is based on ionic interactions between the binding entity (target protein or impurity) and the functional group immobilized on the chromatographic media. Performance may be a function of the mobile phase, the functional group, and the resin backbone. The use of an anionic exchange material versus a cationic exchange material is based, in part, on the local charges of the protein of interest. Anion exchange chromatography can be used in combination with other chromatographic procedures such as affinity chromatography, size exclusion chromatography, hydrophobic interaction chromatography as well as other modes of chromatography known to the skilled artisan.

In the context of chromatographic separation, a chromatographic column is used to house chromatographic support material (resin or solid phase). A sample comprising a protein of interest is loaded onto a particular chromatographic column. The column can then be subjected to one or more wash steps using a suitable wash buffer. Components of a sample that have not adsorbed onto the resin will likely flow through the column. Components that have adsorbed to the resin can be differentially eluted using an appropriate elution buffer.

An anionic agent may be selected from the group consisting of acetate, chloride, formate and combinations thereof. A cationic agent may be selected from the group consisting of Tris, arginine, sodium and combinations thereof. A buffer may be selected from the group consisting of pyridine, piperazine, L-histidine, Bis-Tris, Bis-Tris propane, imidazole, N-ethylmorpholine, TEA (triethanolamine), Tris, morpholine, N-methyldiethanolamine, AMPD (2-amino-2-methyl-1,3-propanediol), diethanolamine, ethanolamine, AMP (2-amino-2-methyl-1-propaol), piperazine, 1,3-diaminopropane and piperidine.

A packed anion-exchange chromatography column, anion-exchange membrane device, anion-exchange monolithic device, or depth filter media can be operated either in bind-elute mode, flowthrough mode, or a hybrid mode wherein proteins exhibit binding to the chromatographic material and yet can be washed from such material using a buffer that is the same or substantially similar to the loading buffer.

In the bind-elute mode, a column or membrane device is first conditioned with a buffer with appropriate ionic strength and pH under conditions where certain proteins will adsorb to the resin-based matrix. For example, during the feed load, a protein of interest can be adsorbed to the resin due to electrostatic attraction. After washing the column or the membrane device with the equilibration buffer or another buffer with a different pH and/or conductivity, the product recovery is achieved by increasing the ionic strength (i.e., conductivity) of the elution buffer to compete with the solute for the charged sites of the anion exchange matrix. Changing the pH and thereby altering the charge of the solute is another way to achieve elution of the solute. The change in conductivity or pH may be gradual (gradient elution) or stepwise (step elution).

In the flowthrough mode, a column or membrane device is operated at a selected pH and conductivity such that the protein of interest does not bind to the resin or the membrane while the acidic species will either be retained on the column or will have a distinct elution profile as compared to the protein of interest. In the context of this strategy, acidic species will interact with or bind to the chromatographic material under suitable conditions while the protein of interest and certain aggregates and/or fragments of the protein of interest will flow through the column.

In some aspects, an AEX step is performed in negative mode (flowthrough mode), where negatively charged process related impurities are adsorbed to the immobilize, positively charged ligand, and the protein of interest flows through.

In some aspects, a pH of a sample loaded onto an AEX column (“load pH”) may be selected to reduce esterase or lipase activity in the sample. In some aspects, an AEX load pH may be about 7.8, about 7.9, about 8.0, about 8.1, about 8.2, about 8.3, between about 7.8 and about 8.3, between about 7.8 and about 8.0, between about 8.0 and about 8.3, or between about 8.0 and about 8.2.

Non-limiting examples of anionic exchange resins include diethylaminoethyl (DEAE), quaternary aminoethyl (QAE) and quaternary amine (Q) groups. Additional non-limiting examples include: Poros 50PI and Poros 50HQ, which are a rigid polymeric bead with a backbone consisting of cross-linked poly[styrene-divinylbenzene]; Poros 50XQ; Capto Q Impres and Capto DEAE, which are a high flow agarose bead; Capto Adhere; Q Sepharose Fast Flow; Toyopearl QAE-550, Toyopearl DEAE-650, and Toyopearl GigaCap Q-650, which are a polymeric base bead; Fractogel® EMD TMAE Hicap, which is a synthetic polymeric resin with a tentacle ion exchanger; Sartobind STICR PA nano, which is a salt-tolerant chromatographic membrane with a primary amine ligand; Sartobind Q nano, which is a strong anion exchange chromatographic membrane; CUNO BioCap, which is a zeta-plus depth filter media constructed from inorganic filter aids, refined cellulose, and an ion exchange resin; XOHC, which is a depth-filter media constructed from inorganic filter aid, cellulose, and mixed cellulose esters; and Unosphere Q. In some aspects, a resin is chosen with a relatively larger pore size, for increased surface area exposed to negatively charged species.

Additives such as polyethylene glycol (PEG), detergents, amino acids, sugars, chaotropic agents can be added to enhance the performance of the separation to achieve better separation, recovery and/or product quality.

Size exclusion chromatography (SEC) or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules, which is higher for small macromolecules. Very large macromolecules may not penetrate the pores of the stationary phase at all; and for very small macromolecules, the probability of penetration is close to unity. While components of larger molecular size move more quickly past the stationary phase, components of small molecular size have a longer path length through the pores of the stationary phase and are thus retained longer in the stationary phase.

Analytes eluting from an SEC column may be separated into fractions based on elution time. For example, analytes eluting earlier than the functional form of a protein of interest, for example the monomeric form, may be broadly categorized as high molecular weight (HMW) species. A HMW fraction may be further subdivided into, for example, a very high molecular weight (vHMW) fraction and a dimer fraction (representing the elution time of a dimer of the protein of interest). Analytes eluting later than the functional form of a protein of interest may be broadly categorized as low molecular weight (LMW) species, and may be further subdivided into a LMW fraction and a later tail fraction. Similarly, sample components besides a protein of interest, for example a lipase, may be form higher and lower molecular weight species that can be separated using SEC.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size. In some aspects, SEC may be used to remove HMW species from a sample, for example to remove proteins that have aggregated into HMW species following agitation or heat stress.

The term “hydrophobic interaction media” means a combination of a support structure and a hydrophobic moiety, wherein the hydrophobic moiety is affixed to the support structure. The media can be in the form of chromatography media, e.g., beads or other particles held in a packed bed column format, in the form of a membrane, or in any format that can accommodate a liquid comprising a protein of interest and contaminants. Thus, support structures include agarose beads (e.g., sepharose), silica beads, cellulosic membranes, cellulosic beads, hydrophilic polymer beads, and the like. The hydrophobic moiety is the business end of the media, which binds to hydrophobic molecules and hydrophobic surfaces of proteins. The degree of hydrophobicity of the media can be controlled by selecting the hydrophobic moiety. For example, the following moieties can be affixed to media substrate to produce hydrophobic interaction media of increasing hydrophobicity, i.e., from low hydrophobicity to high hydrophobicity: ether, butyl, octyl, and phenyl. Alkyl groups may be straight chains or branched. For a review of hydrophobic interaction chromatography and media, see Kuczewski et al., “Development of a polishing step using a hydrophobic interaction membrane adsorber with a PER.C6®-derived recombinant antibody,” Biotech. Bioeng. 105(2):296-305 (2010); Roettger and Ladisch, “Hydrophobic interaction chromatography,” Biotechnol Adv. 7(1):15-29 (1989); Shukla and Sanchayita, “Process for purifying proteins in a hydrophobic interaction chromatography flow-through fraction,” U.S. Pat. No. 7,427,659 B2, Sep. 23, 2008; and Müller and Franzreb, “Suitability of commercial hydrophobic interaction sorbents for temperature-controlled protein liquid chromatography under low salt conditions,” J. Chroma. A 1260:88-96 (2012).

Hydrophobic interaction media is employed in a process known as hydrophobic interaction chromatography (HIC) and is used to separate proteins of interest from product and process related contaminants. When the protein of interest is manufactured in and/or purified from host cells, the product and process related contaminants are referred to as host cell proteins (HCP). HCPs from Chinese hamster ovary (CHO) cells, a common biotherapeutic manufacturing host cell, can be referred to as CHOPs (Chinese hamster ovary proteins). In some cases, a mixture containing the protein of interest (POI) and HCPs are applied to the HIC media in a buffer designed to promote binding of hydrophobic groups in the POI to the hydrophobic moiety of the HIC medium. The POI sticks to the HIC media by binding the hydrophobic moiety, and some HCPs fail to bind and come out in the wash buffer. The POI is then eluted using a buffer that promotes dissociation of the POI from the HIC hydrophobic moiety, thereby separating the POI from unwanted HCPs.

In some cases, the HIC hydrophobic moiety preferentially binds some contaminants such as HCPs, and the POI is collected from the HIC flow-through. This disclosure sets forth examples of the use of HIC in a flow-through mode, wherein a population of contaminant HCPs, including an esterase activity, remain bound to the hydrophobic interaction media.

In some cases affinity chromatography designed to bind specific proteins having lipophilic attributes may be employed in lieu of or in concert with HIC. Since some esterases, such as lipases in general, or phospholipases in particular, bind to triglycerides or phospholipids, molecules that mimic those lipids may be used to capture esterases. For example, “myristoylated ADP ribosylating factor 1” (a.k.a. “myrARF1”) can be used to capture a lipase and allow the POI to remain unbound and flow through. To prepare a myrARF1 affinity column, myrARF1 may be bound to Q-sepharose via N-hydroxysuccinimide activation (see Morgan et al., “Identification of phospholipase B from Dictyostelium discoideum reveals a new lipase family present in mammals, flies and nematodes, but not yeast,” Biochem. J. 382: 441-449 (2004)).

### Mass Spectrometry

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some aspects, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS¹), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m z separation functions can be designed so that in one section of the instrument ions are selected, dissociated in an intermediate region, and the product ions are then transmitted to another analyzer for m z separation and data acquisition. In tandem-in-time, mass spectrometer ions produced in the ion source can be trapped, isolated, fragmented, and m z separated in the same physical device.

The peptides identified by the mass spectrometer can be used as surrogate representatives of the intact protein and their post-translational modifications or other modifications.

They can be used for protein characterization by correlating experimental and theoretical MS/MS data, the latter generated from possible peptides in a protein sequence database. The characterization can include, but is not limited to, sequencing amino acids of the protein fragments, determining protein sequencing, determining protein de novo sequencing, locating post-translational modifications, or identifying post-translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some aspects, mass spectrometry can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

### Formulation Excipients

The pharmaceutical formulations of the present disclosure comprise one or more excipients. The term “excipient,” as used herein, means any non-therapeutic agent added to the formulation to provide a desired consistency, viscosity or stabilizing effect.

The pharmaceutical formulations of the present disclosure may also comprise a buffer or buffer system, which serves to maintain a stable pH and to help stabilize the protein of interest. In some aspects, the buffer or buffer system comprises at least one buffer that has a buffering range that overlaps fully or in part the range of pH 5.5 to 6.3. In various aspects, the pH of the formulation is 5.5, 5.6, 5.7, 5.8, 5.9, 6.0, 6.1, 6.2 or 6.3. In some aspects, the formulations have a pH of 5.9+0.3. In some aspects, the formulations have a pH of 5.9+0.2. In some aspects, the formulations have a pH of 5.9+0.1. In certain aspects, the buffer comprises a histidine buffer. In certain aspects, the buffer comprises an acetate buffer. In certain aspects, the buffer (e.g., histidine and/or acetate) is present at a concentration of from about 1 mM to about 40 mM, about 5 mM to about 30 mM, about 10 mM to about 15 mM; or about 15 mM to about 25 mM. In some aspects, the buffer includes a histidine buffer at a concentration of from 15 mM to 25 mM. In some aspects, the buffer includes a histidine buffer at a concentration of 20 mM±2 mM. In some cases, the histidine buffer is present at a concentration of 15 mM, 16 mM, 17 mM, 18 mM, 19 mM, 20 mM, 21 mM, 22 mM, 23 mM, 24 mM, or 25 mM. In some aspects, the buffer comprises an acetate buffer at a concentration of from 10 mM to 15 mM. In some aspects, the buffer comprises an acetate buffer at a concentration of 12.5 mM #1.25 mM. In some cases, the acetate buffer is present at a concentration of 10 mM, 10.5 mM, 11 mM, 11.5 mM, 12 mM, 12.5 mM, 13 mM, 13.5 mM, 14 mM, 14.5 mM, or 15 mM. In some aspects, the formulations of the present disclosure include both histidine and acetate buffers at any of the concentrations discussed above. In some cases, the formulations contain a histidine buffer at a concentration of from 15 mM to 25 mM, and an acetate buffer at a concentration of from 10 mM to 15 mM. In some cases, the formulations contain a histidine buffer at a concentration of 20 mM±2 mM, and an acetate buffer at a concentration of 12.5 mM±1.25 mM.

The pharmaceutical formulations of the present disclosure may also comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some aspects, the sugar is sucrose. In some cases, the sugar (e.g., sucrose) acts as a thermal stabilizer for the protein of interest.

The amount of sugar (e.g., sucrose) contained within the pharmaceutical formulations of the present disclosure will vary depending on the specific circumstances and intended purposes for which the formulations are used. In certain aspects, the formulations may contain about 0.1% to about 20% sugar; about 0.5% to about 20% sugar; about 1% to about 20% sugar; about 2% to about 15% sugar; about 3% to about 8% sugar; or about 4% to about 6% sugar. For example, the pharmaceutical formulations of the present disclosure may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 15%; or about 20% sugar (e.g., sucrose). In some aspects, the formulations contain about 5% sugar (e.g., sucrose). In some aspects, the formulations contain about 5%+0.5% sugar (e.g., sucrose). Each of the percentages noted above corresponds to a percent weight/volume (w/v).

In certain aspects, the pharmaceutical formulations of the disclosure comprise at least one amino acid. In some aspects, the amino acid is arginine. In some aspects, the arginine is provided in the form of arginine hydrochloride. In some cases, the amino acid (e.g., arginine) acts as a viscosity modifier for the formulations of the protein of interest.

The amount of amino acid contained within the pharmaceutical formulations of the present disclosure may vary depending on the specific properties desired of the formulations, as well as the particular circumstances and purposes for which the formulations are intended to be used. In certain aspects, the formulations may contain about 1 mM to about 200 mM of an amino acid; about 5 mM to about 150 mM of an amino acid; about 10 mM to about 100 mM of an amino acid; about 20 mM to about 80 mM of an amino acid; about 20 mM to about 30 mM of an amino acid; about 45 mM to about 55 mM of an amino acid; or about 70 mM to about 80 mM of an amino acid. For example, the pharmaceutical formulations of the present disclosure may comprise about 5 mM; about 10 mM; about 15 mM; about 20 mM; about 25 mM; about 30 mM; about 35 mM; about 40 mM; about 45 mM; about 50 mM; about 55 mM; about 60 mM; about 65 mM; about 70 mM; about 75 mM; about 80 mM; about 85 mM; about 90 mM; about 95 mM; or about 100 mM of an amino acid (e.g., arginine). In some aspects, the formulations contain about 25 mM of an amino acid (e.g., arginine). In some aspects, the formulations contain about 50 mM of an amino acid (e.g., arginine). In some aspects, the formulations contain about 75 mM of an amino acid (e.g., arginine).

The pharmaceutical formulations of the present disclosure may also comprise one or more organic cosolvents in a type and in an amount that stabilizes the protein of interest under conditions of rough handling or agitation, such as, e.g., orbital shaking. In some aspects, the organic cosolvent is a surfactant. As used herein, the term “surfactant” means a substance which reduces the surface tension of a fluid in which it is dissolved and/or reduces the interfacial tension between oil and water. Surfactants can be ionic or non-ionic. Specific non-ionic surfactants that can be included in the formulations of the present disclosure include, for examples, polysorbates such as PS20 and PS80, poloxamers such as poloxamer 188, and polyethylene glycols (PEGs) such as PEG3350.

The amount of surfactant contained within the pharmaceutical formulations of the present disclosure may vary depending on the specific properties desired of the formulations, as well as the particular circumstances and purposes for which the formulations are intended to be used. In certain aspects, the formulations may contain at least about 0.01% surfactant. In certain aspects, the formulations may contain less than 0.2% surfactant. In some aspects, the formulations may contain less than 0.5% surfactant. In certain aspects, the formulations may contain about 0.01% to about 0.49% surfactant; about 0.01% to about 0.39% surfactant; about 0.01% to about 0.29% surfactant; about 0.01% to about 0.19% surfactant; about 0.01% to about 0.15% surfactant; about 0.01% to about 0.12%; about 0.01% to about 0.11% surfactant; about 0.01% to about 0.1% surfactant; or about 0.01% to about 0.09% surfactant. For example, the formulations of the present disclosure may comprise about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; about 0.30%; about 0.35%; about 0.40%; about 0.45%; or about 0.50% surfactant (e.g., PS20, PS80, poloxamer 188 or PEG3350). In some aspects, the formulations contain about 0.01% to 0.19% poloxamer 188. In some aspects, the formulations contain about 0.01% to about 0.49% poloxamer 188. In some aspects, the formulations contain about 0.01% to 0.19% PEG3350. In some aspects, the formulations contain about 0.01% to 0.49% PEG3350. Each of the percentages noted above corresponds to a percent weight/volume (w/v).

### Polysorbates

In some aspects, the surfactant in the composition can be a polysorbate. As used herein, “polysorbate” refers to a common excipient used in formulation development to protect antibodies against various physical stresses such as agitation, freeze-thaw processes, and air/water interfaces (Emily Ha, Wei Wang & Y. John Wang, Peroxide formation in polysorbate 80 and protein stability, 91 JOURNAL OF PHARMACEUTICAL SCIENCES 2252-2264 (2002); Bruce A. Kerwin, Polysorbates 20 and 80 Used in the Formulation of Protein Biotherapeutics: Structure and Degradation Pathways, 97 JOURNAL OF PHARMACEUTICAL SCIENCES 2924-2935 (2008); Hanns-Christian Mahler et al., Adsorption Behavior of a Surfactant and a Monoclonal Antibody to Sterilizing-Grade Filters, 99 Journal of Pharmaceutical Sciences 2620-2627 (2010)) and can include a non-ionic, amphipathic surfactant composed of fatty acid esters of polyoxyethylene-sorbitan. The esters can include polyoxyethylene sorbitan head group and either a saturated monolaurate side chain (polysorbate 20; PS20) or an unsaturated monooleate side chain (polysorbate 80; PS80). In some aspects, the polysorbate can be present in the formulation in the range of about 0.001% to 1% (weight/volume). Polysorbate can also contain a mixture of various fatty acid chains; for example, polysorbate 80 contains oleic, palmitic, myristic and stearic fatty acids, with the monooleate fraction making up approximately 58% of the polydisperse mixture (Nitin Dixit et al., Residual Host Cell Protein Promotes Polysorbate 20 Degradation in a Sulfatase Drug Product Leading to Free Fatty Acid Particles, 105 JOURNAL OF PHARMACEUTICAL SCIENCES 1657-1666 (2016)). Non-limiting examples of polysorbates include polysorbate-20, polysorbate-40, polysorbate-60, polysorbate-65, and polysorbate-80.

A polysorbate can be susceptible to auto-oxidation in a pH- and temperature-dependent manner, and additionally, exposure to UV light can also produce instability (Ravuri S. k. Kishore et al., Degradation of Polysorbates 20 and 80: Studies on Thermal Autoxidation and Hydrolysis, 100 JOURNAL OF PHARMACEUTICAL SCIENCES 721-731 (2011)), resulting in free fatty acids in solution along with the sorbitan head group. The free fatty acids resulting from polysorbate can include any aliphatic fatty acids with six to twenty carbons. Non-limiting examples of free fatty acids include oleic acid, palmitic acid, stearic acid, myristic acid, lauric acid, or combinations thereof.

In some exemplary aspects, the polysorbate can form free fatty acid particles. The free fatty acid particles can be at least about 1 μm in size or at least about 5 μm in size. Further, these fatty acid particles can be classified according to their size as visible (about >100 μm), sub-visible (about ≤100 μm, which can be sub-divided into micron (1-100 μm) and submicron (100 nm-1000 nm)) and nanometer particles (about ≤100 nm) (Linda Narhi, Jeremy Schmit & Deepak Sharma, Classification of protein aggregates, 101 JOURNAL OF PHARMACEUTICAL SCIENCES 493-498). In some exemplary aspects, the fatty acid particles can be visible particles. Visible particles can be determined by visual inspection. In some aspects, the fatty acid particles can be sub-visible particles. Subvisible particles can be monitored by the light blockage method according to United States Pharmacopeia (USP). An increase in fatty acid particles may cause a product to no longer be of acceptable quality, and therefore a rate of increase of fatty acid particles may be used as a measure of product shelf life. Fatty acid particles may form when free fatty acids are released into a formulation and exceed a concentration at which they are soluble, thereby precipitating out of solution. Therefore, measuring a degradation of polysorbate or a concentration of released free fatty acids may be indicators of the formation of fatty acid particles, and by extension of predicted product shelf life. Additionally, preventing a degradation of polysorbate, a release of free fatty acids, and/or a formation of fatty acid particles may be important for extending product shelf life and improving product quality.

In some exemplary aspects, the concentration of polysorbate in the formulation can be about 0.001% w/v, about 0.002% w/v, about 0.003% w/v, about 0.004% w/v, about 0.005% w/v, about 0.006% w/v, about 0.007% w/v, about 0.008% w/v, about 0.009% w/v, about 0.01% w/v, about 0.015% w/v, about 0.02% w/v, 0.025% w/v, about 0.03% w/v, about 0.035% w/v, about 0.04% w/v, about 0.045% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, or about 1% w/v. In one aspect, the concentration of polysorbate in the formulation is about 1% w/v.

In some exemplary aspects, the concentration of free fatty acids in the formulation can be about 10 ng/ml, about 20 ng/mL, about 30 ng/mL, about 40 ng/ml, about 50 ng/ml, about 60 ng/mL, about 70 ng/mL, about 80 ng/mL, about 90 ng/mL, about 100 ng/ml, about 200 ng/ml, about 300 ng/ml, about 400 ng/mL, about 500 ng/mL, about 600 ng/mL, about 700 ng/mL, about 800 ng/mL, about 900 ng/ml, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 20 μg/mL, about 30 μg/mL, or about 40 μg/mL.

In some exemplary aspects, the polysorbate can be degraded by a host cell protein present in the composition. In some aspects, the host cell protein may be an esterase or lipase. Residual esterase or lipase activity in a formulation may be indirectly assessed by measuring polysorbate degradation, release of free fatty acids, or a concentration of visible or subvisible fatty acid particles.

The term “fatty acid ester” means any organic compound that contains a fatty acid chain linked to a head group via an ester bond. An ester bond is formed when a hydroxyl group (e.g., an alcohol or carboxylic acid) is replaced by an alkoxy group. As used herein, the hydroxyl group can be part of a carboxylic acid, more specifically a fatty acid, and/or an alcohol, such as glycerol, sorbitol, sorbitan, isosorbide, or the like. The alcohol group is generally referred to herein as the head group.

Examples of fatty acid esters generally include phospholipids, lipids (e.g., the head group is glycerol, including monoglycerides, diglycerides, and triglycerides), and surfactants and emulsifiers, including for example polysorbates like polysorbate 20, polysorbate 60, and polysorbate 80, which are non-ionic detergents. Surfactants and emulsifiers are useful as cosolvents and stabilizers. They function by associating with both a hydrophilic surface and a lipophilic surface to maintain dispersion and structural stability of ingredients, like proteins. Surfactants are added to protein formulations primarily to enhance protein stability against mechanical stress, such as air/liquid interface and solid/liquid interface shear. Without a surfactant, proteins may in some cases become structurally unstable in solution, and form multimeric aggregates that eventually become subvisible particles.

The term “fatty acid” or “fatty acid chain” means a carboxylic acid having an aliphatic tail. An aliphatic tail is simply a hydrocarbon chain comprising carbon and hydrogen, and in some cases, oxygen, sulfur, nitrogen and/or chlorine substitutions. Aliphatic tails can be saturated (as in saturated fatty acids), which means that all carbon-carbon bonds are single bonds (i.e., alkanes). Aliphatic tails can be unsaturated (as in unsaturated fatty acids), wherein one or more carbon-carbon bonds are double bonds (alkenes), or triple bonds (alkynes).

Fatty acids are generally designated as short-chain fatty acids, which have fewer than six carbons in their aliphatic tails, medium-chain fatty acids having six to twelve carbons, long-chain fatty acids having thirteen to twenty one carbons, and very long chain fatty acids having aliphatic tails of twenty two carbons and longer. As mentioned above, fatty acids are also categorized according to their degree of saturation, which correlates to stiffness and melting point. Common fatty acids include caprylic acid (8 carbons:0 double bonds; 8:0), capric acid (10:0), lauric acid (12:0), myristic acid (14:0), myristoleic acid (14:1), palmitic acid (16:0), palmitoleic acid (16:1), sapienic acid (16:1), stearic acid (18:0), oleic acid (18:1), elaidic acid (18:1), vaccenic acid (18:1), linoleic acid (18:2), linelaedic acid (18:2), alpha-linolenic acid (18:3), arachidic acid (20:0), arachidonic acid (20:4), eicosapentaenoic acid (20:5), behenic acid (22:0), erucic acid (22:1), docosahexaenoic acid (22:6), lignoceric acid (24:0), and cerotic acid (26:0).

As mentioned above, polysorbates are fatty acid esters useful as non-ionic surfactants and protein stabilizers. Polysorbate 20, polysorbate 40, polysorbate 60, and polysorbate 80 are widely employed in the pharmaceutical, cosmetic, and food industries as stabilizers and emulsifiers. Polysorbate 20 mostly comprises the monolaurate ester of polyoxyethylene (20) sorbitan. Polysorbate 40 mostly comprises the monopalmitate ester of polyoxyethylene (20) sorbitan. Polysorbate 60 mostly comprises the monostearate ester of polyoxyethylene (20) sorbitan. Polysorbate 80 mostly comprises the monooleate ester of polyoxyethylene (20) sorbitan.

The quality of commercial grades of polysorbates varies from vendor to vendor. Polysorbates therefore are often mixtures of various chemical entities, consisting mostly of polyoxyethylene (20) sorbitan monoesters (as described above) with, in some cases, isosorbide ester contaminants. The head group (in this case polyoxyethylene (20) sorbitan) comprises a sorbitan (a mixture of dehydrated sorbitols, including 1,4-anhydrosorbitol, 1,5-anhydrosorbitol, and 1,4,3,6-dianhydrosorbitol) substituted at three of its alcohol groups to form ether bonds with three polyoxyethylene groups. The fourth alcohol group is substituted with a fatty acid to form a fatty acid ester.

In some commercially available batches of polysorbates, the polysorbate contains isosorbide monoesters. Isosorbide is a heterocyclic derivative of glucose, also prepared by the dehydration of sorbitol. It is a diol, i.e., having two alcohol groups that can take part in the formation of one or two ester bonds. Thus, for example, some lots of polysorbate 20 can contain significant amounts of isosorbide laurate mono- and di-esters, and some lots of polysorbate 80 can contain significant amounts of isosorbide oleate mono- and di-esters.

In addition to head group variation, preparations of polysorbates contain variable amounts of other fatty acid esters. For example, an analysis of one particular source of polysorbate 20 revealed<10% caprylic acid, <10% capric acid, 40-60% lauric acid, 14-25% myristic acid, 7-15% palmitic acid, <11% oleic acid, <7% stearic acid, and <3% linoleic acid. An analysis of a polysorbate 80 batch revealed<5% myristic acid, <16% palmitic acid, >58% oleic acid, <6% stearic acid, and <18% linoleic acid. An analysis of another source of polysorbate 80 revealed about 70% oleic acid, with the remainder being other fatty acid esters and impurities. An analysis of yet another source of polysorbate 80 revealed about 86-87% oleic acid. An analysis of a further, more recently-developed source of polysorbate 80 revealed≥99% oleic acid.

In some aspects, a concentration of oleic acid in polysorbate 80 may be between about 50% and about 100%, between about 58% and about 100%, between about 60% and 100%, between about 80% and about 100%, between about 90% and about 100%, between about 95% and about 100%, between about 98% and about 100%, between about 99% and about 100%, between about 98.0% and about 99.9%, between about 98.5% and about 99.5%, between about 99.0% and about 99.9%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 99%, at least 99.5%, about 58%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 98.5%, about 99%, about 99.1%, about 99.2%, about 99.3%, about 99.4%, about 99.5%, about 99.6%, about 99.7%, about 99.8%, about 99.9%, or about 100%.

Biopharmaceutical drugs are often formulated with non-ionic detergents like polysorbate 20 or polysorbate 80. These detergents help stabilize large molecules like antibodies and other proteins, and help prevent the formation of supermolecular ternary complexes or other aggregates. Aggregates can become nanoparticles or subvisible particles in the 10 to 100 micron range or 2 to 100 micron range, and interfere with drug product stability and shelf-life. Therefore, the stability of protein formulations depends in some cases upon the stability of the non-ionic detergent additive. However, and as is further discussed herein, polysorbate 20 and polysorbate 80 can, in some instances, contribute to the formation of aggregates, nanoparticles, and subvisible particles.

The phrase “subvisible particle” means a particle that is not visible, especially in a liquid. In other words, a solution or other liquid containing subvisible particles, but not visible particles, will not appear cloudy. Subvisible particles generally include those particles 100 microns or less in diameter, but in some cases include particles under 150 microns (Narhi et al., “A critical review of analytical methods for subvisible and visible particles,” Curr Pharm Biotechnol 10(4):373-381 (2009)). Subvisible particles may be the result of foreign contaminants or protein aggregation. Protein aggregates can be soft and amorphous in shape and therefore may be difficult to detect using conventional light obscuration and microscopic methods (Singh and Toler, “Monitoring of subvisible particles in therapeutic proteins,” Methods Mol Biol. 2012; 899:379-401). Subvisible particles may comprise, inter alia, silicone contaminants (oily droplets), free fatty acids (oily droplets), aggregated protein (amorphous particles), and/or protein/fatty acid complexes (amorphous particles).

Subvisible particles can be detected by any one or more of various methods. The USP standards specify light obscuration and optical microscopy protocols. Other methods include micro-flow image (MFI) analysis, Coulter counting, and submicron particle tracking methods. For light obscuration (LO), particles are counted based on the shadows they cast upon a light detector as they pass through a light beam in a flow cell. The size, shape and inverse intensity of the shadow depends upon the size, shape and difference in the refractive index of the particle relative to the solution. The lower size range for detection using LO is about 2 microns. A commonly used LO device is the HIAC instrument (Beckman Coulter, Brea, Calif.). Several methods of measurement and characterization of SVPs (e.g., light obscuration, flow microscopy, the electrical sensing zone method, and flow cytometry), are discussed in, for example, Narhi et al., “Subvisible (2-100 μm) Particle Analysis During Biotherapeutic Drug Product Development: Part 1, Considerations and Strategy,” J. Pharma. Sci. 104:1899-1908 (2015).

Light obscuration is criticized for underestimating protein aggregates and other amorphous structures. Flow image analysis, such as micro-flow imaging (MFI) (Brightwell Technologies, Ottawa, Ontario), is a more sensitive method of detecting the irregularly shaped, fragile, and transparent proteinaceous subvisible particles, and of differentiating those types of particles from silicone micro-droplets, air bubbles, and other foreign contaminants (Sharma et al., “Micro-flow imaging: Flow microscopy applied to sub-visible particulate analysis in protein formulations,” AAPS J. 12(3): 455-464 (2010)). In general, because SVP measurement and characterization by light obscuration analysis is less sensitive than MFI, particle counts detected by MFI will tend to be higher than particle counts detected by light obscuration analysis. Briefly, MFI is flow microscopy in which successive bright field images are taken and analyzed in real time. Image analysis algorithms are applied to the images to discriminate air bubbles, silicone oil droplets, and proteinaceous aggregates. Volumes as low as about 250 microliters to as high as tens of milliliters can be analyzed. Depending on the system used, particles in the range of two to 300 microns, or one to 70 microns can be detected (Id).

In some aspects, visible or subvisible particles in a formulation can be detected and analyzed by Raman spectroscopy. As used herein, the term “Raman spectroscopy” refers to a spectroscopic method based on Raman scattering method. Raman spectroscopy can provide a Raman spectrum, which can identify the presence and position of bands in the fingerprint region (2000 to 400 cm-1) which enables the chemical identification of the analyzed material by comparison with a database of Raman spectra (C. V. Raman and K. S. Krishnan, A new type of secondary radiation, 121 NATURE 501-502 (1928); Zai-Qing Wen, Raman spectroscopy of protein pharmaceuticals, 96 JOURNAL OF PHARMACEUTICAL SCIENCES 2861-287 (2007)).

The FDA and other government regulatory agencies have placed limits on the amount of subvisible particles allowed in parenteral drug formulations. The major articulated concern is the uncertainty surrounding potential immunogenicity and downstream negative effects in the patient receiving the drug (Singh et al., “An industry perspective on the monitoring of subvisible particles as a quality attribute for protein therapeutics,” J. Pharma. Sci. 99(8):3302-21 (2010)). For small volume parenteral drugs (e.g., 100 mL or below), the pharmacopeia limits subvisible particles (SVP) of greater than or equal to 10 microns to no more than 6,000 SVPs per container, and SVPs of greater than or equal to 25 microns to no more than 600 per container, when determined by light obscuration analysis; and SVPs of greater than or equal to 10 microns to no more than 3,000 SVPs per container, and SVPs of greater than or equal to 25 microns to no more than 300 per container, when determined by the membrane microscopic test. (United States Pharmacopeia and National Formulary (USP 40-NF 28), <787>Subvisible Particulate Matter in Therapeutic Protein Injections.) For ophthalmic drugs, the SVP limits are 50 per mL of 10 microns or greater, 5 per mL of 25 microns or greater, and 2 per mL of 50 microns or greater (Id. at <78922 Particulate Matter in Ophthalmic Solutions). Regulatory agencies are increasingly contemplating that manufacturers establish specifications for SVPs of 2 microns or greater (see Singh et al., “An industry perspective on the monitoring of subvisible particles as a quality attribute for protein therapeutics,” J. Pharm. Sci. 99(8):3302-21 (2010)).

The term “esterase” means an enzyme that catalyzes the hydrolysis of an ester bond to create an acid and an alcohol. Esterases are a diverse category of enzymes, including acetyl esterases (e.g., acetylcholinesterase), phosphatases, nucleases, thiolesterases, lipases and other carboxyl ester hydrolases. As its name implies, a carboxyl ester hydrolase (a.k.a. carboxylesterase, carboxylic-ester hydrolase, and EC 3.1.1.1) uses water to hydrolyze a carboxylic ester into an alcohol and a carboxylate. A lipase is a carboxyl ester hydrolase that catalyzes the hydrolysis of lipids, including triglycerides, fats and oils into fatty acids and an alcohol head group. For example, triglycerides are hydrolyzed by lipases like pancreatic lipase to form monoacylglycerol and two fatty acid chains.

Phospholipases are lipases that hydrolyze phospholipids into fatty acids and other products. Phospholipases fall into four broad categories: phospholipase A (including phospholipase A1 and phospholipase A2), phospholipase B, and the phosphodiesterases phosphodiesterase C and phosphodiesterase D. In addition to the canonical phospholipases, phospholipase B-like enzymes, which reside at the lysosome lumen, are thought to be involved in lipid catalysis. For example, phospholipase B-like 2 (PLBL2) is postulated to have esterase activity based upon sequence homology and subcellular localization (Jensen et al., “Biochemical characterization and liposomal localization localization of the mannose-6-phosphate protein p76,” Biochem. J. 402: 449-458 (2007)).

As used herein, the phrase “percent fatty ester hydrolysis” means the molar proportion of fatty acid ester that has been hydrolyzed. Since hydrolysis of a fatty acid ester results in the release of a free fatty acid, the percent fatty ester hydrolysis can be determined by measuring the free fatty acid in a sample. Therefore, percent fatty ester hydrolysis may be determined by calculating moles of free fatty acid divided by the sum of moles of fatty acid plus moles of fatty acid ester. In the case of percent hydrolysis of polysorbate 80 or polysorbate 20, that number may be determined by calculating the moles of free fatty acid and dividing by the total moles of remaining polysorbate plus moles of free fatty acid.

The term “esterase inhibitor” means any chemical entity that reduces, inhibits, or blocks the activity of an esterase. The applicants envision that the inclusion of an esterase inhibitor in a protein formulation containing a fatty acid ester surfactant may help maintain protein stability and fatty acid ester stability and help reduce SVP formation. Common esterases known in the art include orlistat (tetrahydrolipistatin; an inhibitor of carboxylesterase 2 and lipoprotein lipase), diethylumbelliferyl phosphate (a cholesterol esterase [lipsase A] inhibitor), URB602 ([1-1′-biphenyl]-3-t1-carbamicacid cyclohexyl ester; a monoacylglycerol lipase inhibitor), and 2-butoxyphenylboronic acid (an inhibitor of hormone-sensitive lipase). The inclusion of an esterase inhibitor during purification of a protein of interest or in the final formulation is expected to prevent or slow the hydrolysis of non-ionic detergents like polysorbate 20 and polysorbate 80, which in turn is expected to prevent or reduce subvisible particle formation.

### Exemplary Formulations

According to one aspect of the present disclosure, the pharmaceutical formulation comprises: (i) a human antibody that specifically binds to hIL-4R (e.g., an antibody comprising one or more sequences disclosed in Table 1 below); (ii) acetate; (iii) histidine; (iv) sucrose; (v) arginine; and (v) a surfactant comprising a polyethylene glycol or a poloxamer.

In some cases, the stable liquid pharmaceutical formulation comprises: (i) a human antibody that specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1. In another aspect, the surfactant comprising a polyethylene glycol or a poloxamer may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1. In another aspect, the surfactant comprising a polyethylene glycol or a poloxamer may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1. In another aspect, the surfactant comprising PEG3350 or poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 25 mM±2.5 mM; and (vi) a surfactant comprising PEG3350 at a concentration of from 0.01% w/v to 0.19% w/v, wherein the formulation has a pH of 5.9+0.2. In another aspect, the surfactant comprising PEG3350 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v+0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9+0.2. In another aspect, the surfactant comprising poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1. In another aspect, the surfactant comprising polyethylene glycol or a poloxamer may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1. In another aspect, the PEG3350 or poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 25 mM±2.5 mM; and (vi) a surfactant comprising PEG3350 at a concentration of from 0.01% w/v to 0.19% w/v, wherein the formulation has a pH of 5.9+0.2. In another aspect, the PEG3350 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9+0.2. In another aspect, the poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 150 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1. In another aspect, the surfactant comprising polyethylene glycol or a poloxamer may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 150 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1. In another aspect, the surfactant comprising PEG3350 or poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 150 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 25 mM±2.5 mM; and (vi) a surfactant comprising PEG3350 at a concentration of from 0.01% w/v to 0.19% w/v, wherein the formulation has a pH of 5.9+0.2. In another aspect, the surfactant comprising PEG3350 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 150 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v+0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9+0.2. In another aspect, the surfactant comprising poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1. In another aspect, the surfactant comprising polyethylene glycol or a poloxamer may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1. In another aspect, the surfactant comprising PEG3350 or poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 25 mM±2.5 mM; and (vi) a surfactant comprising PEG3350 at a concentration of from 0.01% w/v to 0.19% w/v, wherein the formulation has a pH of 5.9+0.2. In another aspect, the surfactant comprising PEG3350 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v+0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9+0.2. In another aspect, the surfactant comprising poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v+0.5% w/v; (v) arginine at a concentration of 50 mM±2.5 mM; and (vi) a surfactant comprising PEG3350 at a concentration of from 0.01% w/v to 0.19% w/v, wherein the formulation has a pH of 5.9+0.2. In another aspect, the surfactant comprising PEG3350 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 50 mM±2.5 mM; and (vi) a surfactant comprising poloxamer 188 at a concentration of from 0.01% w/v to 0.19% w/v, wherein the formulation has a pH of 5.9+0.2. In another aspect, the surfactant comprising poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v=0.5% w/v; (v) arginine at a concentration of 75 mM±2.5 mM; and (vi) a surfactant comprising PEG3350 at a concentration of from 0.01% w/v to 0.19% w/v, wherein the formulation has a pH of 5.9+0.2. In another aspect, the surfactant comprising PEG3350 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In some cases, the stable liquid pharmaceutical formulation comprises: (i) a human antibody at a concentration of 175 mg/mL+10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v+0.5% w/v; (v) arginine at a concentration of 75 mM±2.5 mM; and (vi) a surfactant comprising poloxamer 188 at a concentration of from 0.01% w/v to 0.19% w/v, wherein the formulation has a pH of 5.9+0.2. In another aspect, the surfactant comprising poloxamer 188 may be in a concentration of from about 0.01% w/v to 0.5% w/v, or about 0.01% w/v to 0.4%, or about 0.01% w/v to 0.3%, or about 0.01% w/v to 0.2%. In another aspect, the pH of the formulation can be about 5.4 to 6.5, about 5.5 to 6.2 or about 5.6 to 6.2.

In any of the various aspects of the pharmaceutical formulations discussed above or herein, the human IL-4R antibody may comprise a human IgG1 heavy chain constant region.

In any of the various aspects of the pharmaceutical formulations discussed above or herein, the human IL-4R antibody may comprise a human IgG4 heavy chain constant region.

In some aspects, the human IL-4R antibody may comprise a heavy chain comprising the amino acid sequence of SEQ ID NO: 9 and a light chain comprising the amino acid sequence of SEQ ID NO: 10.

Additional non-limiting examples of pharmaceutical formulations encompassed by the present disclosure are set forth elsewhere herein, including the working Examples presented below.

### Stability of the Pharmaceutical Formulations

The pharmaceutical formulations of the present disclosure exhibit high levels of stability. The term “stable,” as used herein in reference to the pharmaceutical formulations, means that the proteins of interest within the pharmaceutical formulations retain an acceptable degree of structure and/or function and/or biological activity after storage for a defined amount of time. A formulation may be stable even though the protein contained therein does not maintain 100% of its structure and/or function and/or biological activity after storage for a defined amount of time. Under certain circumstances, maintenance of about 90%, about 95%, about 96%, about 97%, about 98% or about 99% of a protein's structure and/or function and/or biological activity after storage for a defined amount of time may be regarded as “stable.”

Stability can be measured, inter alia, by determining the percentage of protein that forms an aggregate within the formulation after storage for a defined amount of time at a defined temperature, or under stress conditions (e.g., agitation), wherein stability is inversely proportional to the percent aggregate that is formed. The percentage of aggregated protein can be determined by, inter alia, size exclusion chromatography (e.g., size exclusion high performance liquid chromatography (SE-HPLC) or size exclusion ultra-performance liquid chromatography (SE-UPLC)). An “acceptable degree of stability”, as that phrase is used herein, means that at most about 15%, 10%, 5%, 4%, 3%, 2.5%, 2%, 1.5%, 1%, 0.5%, or 0.1% of the protein can be detected in an aggregate in the formulation after storage for a defined amount of time at a given temperature, or under specified stress conditions. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4° C.-8° C., about 5° C., about 25° C., about 35° C., about 37° C. or about 45° C. The “stress condition” to which the formulated protein of interest may be subjected may be agitation stress (e.g., vortexing) for a period of 10 minutes, 20 minutes, 30 minutes, 40 minutes, 50 minutes, 60 minutes, 70 minutes, 80 minutes, 90 minutes, 100 minutes, 110 minutes, 120 minutes, 150 minutes, 180 minutes, or more. For example, a pharmaceutical formulation comprising an anti-IL-4R antibody may be deemed stable if after nine months of storage at 5° C., less than about 2%, 1.75%, 1.5%, 1.25%, 1%, 0.75%, 0.5%, 0.25%, or 0.1% of the antibody is detected in an aggregated form. A pharmaceutical formulation may also be deemed stable if after 56 days of storage at 45° C., less than about 12% of the protein is detected in an aggregated form. A pharmaceutical formulation may also be deemed stable if after 42 days of storage at 45° C., less than about 10% or less than about 9% of the protein is detected in an aggregated form. A pharmaceutical formulation may also be deemed stable if after 28 days of storage at 45° C., less than about 8% or less than about 7.5% or less than about 7% of the protein is detected in an aggregated form. A pharmaceutical formulation may also be deemed stable if after 14 days of storage at 45° C., less than about 6% of the protein is detected in an aggregated form. A pharmaceutical formulation may also be deemed stable if after three months of storage at −20° C., −30° C., or −80° C. less than about 2%, 1.9%, 1.8%, 1.7%, 1.6%, 1.5%, 1%, 0.5%, or 0.1% of the protein is detected in an aggregated form. A pharmaceutical formulation may also be deemed stable if after 120 minutes of agitation (e.g., via vortexing) at room temperature less than 3% or less than 2.5% of the protein is detected in an aggregated form.

Stability can also be measured, inter alia, by determining particulate formulation within the formulation after storage for a defined amount of time at a defined temperature. Particle formation can be determined, for example, by microscopy techniques or by micro-flow imaging techniques.

In some aspects, the formulations of the present disclosure comprise a detectable amount of a lipase (for example, PLBL2). Methods of detecting and quantifying the presence and activity of phospholipase are known in the art. In some aspects, the phospholipase is detected by immunoassay (e.g., ELISA). In some aspects, the phospholipase is detected by liquid chromatography-mass spectrometry (LC-MS).

Accordingly, a pharmaceutical formulation (containing an esterase or lipase) may be deemed stable if after storage for a period of time (e.g., 6, 12, 18, 24 or 36 months or more) at a defined temperature (e.g., 5)° ° C., no more than a specified number of fatty acid particles≥ 10 μm or ≥ 25 μm in size (e.g., 3000 particles, 1000 particles, 500 particles, 250 particles, 100 particles, or 50 particles) are identified within a volume of 2.25 mL. For example, a pharmaceutical formulation may be deemed stable if after 24 months of storage at 5° C. no more than 3000 fatty acid particles are identified within a volume of 2.25 mL via microscopy. In another aspect, a pharmaceutical formulation may be deemed stable if after 24 months of storage at 5° C. no more than 1000 fatty acid particles are identified within a volume of 2.25 mL via microscopy. A pharmaceutical formulation may also be deemed stable if after 24 months of storage at 5° C. no more than 500 fatty acid particles are identified within a volume of 2.25 mL via microscopy. A pharmaceutical formulation may also be deemed stable if after 24 months of storage at 5° C. no more than 250 fatty acid particles are identified within a volume of 2.25 mL via microscopy. A pharmaceutical formulation may also be deemed stable if after 24 months of storage at 5° C. no more than 150 fatty acid particles are identified within a volume of 2.25 mL via microscopy. A pharmaceutical formulation may also be deemed stable if after 36 months of storage at 5° C. no more than 1000 fatty acid particles are identified within a volume of 2.25 mL via microscopy. A pharmaceutical formulation may also be deemed stable if after 36 months of storage at 5° C. no more than 500 fatty acid particles are identified within a volume of 2.25 mL via microscopy. A pharmaceutical formulation may also be deemed stable if after 36 months of storage at 5° C. no more than 250 fatty acid particles are identified within a volume of 2.25 mL via microscopy. A pharmaceutical formulation may also be deemed stable if after 36 months of storage at 5° C. no more than 150 fatty acid particles are identified within a volume of 2.25 mL via microscopy. A pharmaceutical formulation may also be deemed stable if after 36 months of storage at 5° C. no more than 100 fatty acid particles are identified within a volume of 2.25 mL via microscopy. A pharmaceutical formulation may also be deemed stable if after 36 months of storage at 5° C. no more than 50 fatty acid particles are identified within a volume of 2.25 mL via microscopy.

Stability can also be measured by, inter alia, determining the percentage of native protein of interest remaining in the formulation after storage for a defined amount of time at a given temperature. The percentage of native protein of interest can be determined by, inter alia, size exclusion chromatography (e.g., size exclusion high performance liquid chromatography (SE-HPLC)). An “acceptable degree of stability,” as that phrase is used herein, means that at least 90% of the native form of the protein can be detected in the formulation after storage for a defined amount of time at a given temperature. In certain aspects, at least about 90%, 91%, 92%, 93%, 94%, 95%, 96%, 97%, 98%, 99% or 100% of the native form of the protein can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4° C.-8° C., about 5° C., about 25° C., about 35° ° C., about 37° C., or about 45° C.

Stability can also be measured, inter alia, by determining the percentage of protein of interest that migrates in a more acidic fraction during ion exchange (“acidic form”) than in the main fraction of protein (“main charge form”), wherein stability is inversely proportional to the fraction of protein in the acidic form. While not wishing to be bound by theory, deamidation of the protein may cause the protein to become more negatively charged and thus more acidic relative to the non-deamidated protein (see, e.g., Robinson, N., Protein Deamidation, PNAS, Apr. 16, 2002, 99(8):5283-5288). The percentage of “acidified” protein can be determined by ion exchange chromatography (e.g., cation exchange high performance liquid chromatography (CEX-HPLC) or cation exchange ultra-performance liquid chromatography (CEX-UPLC)). An “acceptable degree of stability”, as that phrase is used herein, means that at most 50%, 45%, 40%, 35%, 30%, 25%, 20%, 15%, 10%, 5%, 4%, 3%, 2%, 1%, 0.5%, or 0.1% of the protein can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4° C.-8° C., about 5° C., about 25° C., or about 45° C.

Measuring the binding affinity of an antibody of interest to its target may also be used to assess stability. For example, a formulation of the present disclosure may be regarded as stable if, after storage at e.g., −80° C., −30° C., −20° C., 5° C., 25° C., 37° C., 45° C., etc. for a defined amount of time (e.g., 14 days to 9 months), an anti-IL-4R antibody contained within the formulation binds to hIL-4Rα with an affinity that is at least 80%, 85%, 90%, 95%, or more of the binding affinity of the antibody prior to said storage. Binding affinity may be determined by any method, such as e.g., ELISA or plasmon resonance. Biological activity may be determined by, for example, measuring the downstream activity of the IL-4R system in the presence of the antibody, and comparing the activity to the activity of the IL-4R system in the absence of antibody.

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 12 months means that the measurement of stability was taken at or about 12 months after the start of the study.

### Containers and Methods of Administration

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present disclosure including, e.g., clear and opaque (e.g., amber) glass or plastic vials. Likewise, any type of syringe can be used to contain and/or administer the pharmaceutical formulations of the present disclosure. In some aspects, the pharmaceutical formulation is contained in a prefilled syringe (PFS). In some aspects, the pharmaceutical formulation is contained in a prefilled staked needle syringe.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present disclosure, according to certain aspects, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluroTec®,” available from West Pharmaceutical Services, Inc. (Lionville, PA). According to certain aspects of the present disclosure, the pharmaceutical formulations may be contained within a low tungsten syringe that comprises a fluorocarbon-coated plunger. In some aspects, the container is a syringe, such as an Ompi EZ-Fill™ syringe or a BD Neopak™ syringe. In some cases, the syringe is a 1 mL long glass syringe with a 1 mL iWest piston, a 27G thin wall needle and an FM30 needle shield or a BD260 needle shield. In some cases, the syringe is a 2.25 mL glass syringe (e.g., Nuova Ompi). In various aspects, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 ml syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25TM pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park, Ill.), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25 gauge needle. In some cases, subcutaneous injections may be administered using a 27 gauge needle. In some cases, subcutaneous injections may be administered using a 29 gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present disclosure is also contemplated herein. In some cases, the LVD or bolus injector may be configured to inject a medicament into a patient. For example, an LVD or bolus injector may be configured to deliver a “large” volume of medicament (typically about 2 mL to about 10 mL).

The pharmaceutical formulations of the present disclosure can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various aspects, the unit dosage form is contained within a container as discussed herein. Actual dosage levels of the active ingredient (for example, an anti-IL-4R antibody) in the formulations of the present disclosure may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various aspects, the unit dosage form contains an amount of the active ingredient (for example, an anti-IL-4R antibody) intended for a single use. In various aspects, the amount of the active ingredient in the unit dosage form is from about 0.1 mg to about 5000 mg, from about 100 mg to about 1000 mg, and from about 100 mg to about 500 mg, from about 100 mg to about 400 mg, from about 100 mg to about 200 mg, from about 250 mg to about 350 mg, from about 125 mg to about 175 mg, from about 275 mg to about 325 mg, or ranges or intervals thereof. For example, ranges of values using a combination of any of the above recited values (or values contained within the above recited ranges) as upper and/or lower limits are intended to be included. In a particular aspect, the formulation often is supplied as a liquid in unit dosage form. In some aspects, the unit dosage form contains about 100 mg of the active ingredient. In some aspects, the unit dosage form contains about 150 mg. In some aspects, the unit dosage form contains about 200 mg. In some aspects, the unit dosage form contains about 300 mg. In some aspects, the unit dosage form contains about 350 mg. In some aspects, the unit dosage form contains about 600 mg. In some aspects, a unit dosage form according to the present disclosure is suitable for subcutaneous administration to a patient.

The present disclosure also includes methods of preparing a unit dosage form. In one aspect, a method for preparing a pharmaceutical unit dosage form includes combining the formulation of any of foregoing aspects in a suitable container (e.g., those containers discussed herein).

### Therapeutic Uses of the Pharmaceutical Formulations

Pharmaceutical formulations of the present disclosure comprising an anti-IL-4R antibody are useful, inter alia, for the treatment, prevention and/or amelioration of any disease or disorder associated with IL-4R activity.

The therapeutic methods of the present disclosure comprise administering to a subject any formulation comprising an anti-hIL-4R antibody as disclosed herein. The subject to which the pharmaceutical formulation is administered can be, e.g., any human or non-human animal that is in need of such treatment, prevention and/or amelioration, or who would otherwise benefit from the inhibition or attenuation of IL-4R and/or IL-4R-mediated activity. The present disclosure further includes the use of any of the pharmaceutical formulations disclosed herein in the manufacture of a medicament for the treatment, prevention and/or amelioration of any disease or disorder associated with IL-4R activity.

In some aspects, the disease or disorder associated with IL-4R activity is an inflammatory condition, allergic condition, lung/respiratory disorder, gastrointestinal disorder, or dermatological disorder. In some aspects, the disease or disorder is a Type 2 inflammatory disorder. In some aspects, the disease or disorder is an atopic disease. Non-limiting examples of diseases and disorders associated with IL-4R activity include allergy (e.g., food allergy, environmental allergy, grass allergy, peanut allergy, dairy allergy), allergic reactions, allergic bronchopulmonary aspergillosis, allergic fungal rhino-sinusitis (AFRS), allergic rhinitis, alopecia areata, asthma (including mild, moderate, or severe asthma or persistent asthma), arthritis (including septic arthritis), atopic dermatitis (including moderate or severe atopic dermatitis), hand and foot atopic dermatitis, atopic keratoconjunctivitis, autoimmune hemolytic anemia, autoimmune lymphoproliferative syndrome, autoimmune uveitis, Barrett's esophagus, benign prostate hyperplasia, bronchiectasis, bullous pemphigoid, Churg-Strauss syndrome, chronic idiopathic urticaria, cold inducible urticaria, chronic inducible urticaria, chronic spontaneous urticaria (CSU), contact dermatitis (e.g., allergic contact dermatitis), chronic obstructive pulmonary disease (COPD), eosinophilic esophagitis, eosinophilic gastroenteritis, Grave's disease, herpetiformis, hypertrophic scarring, inflammatory bowel disease, Kawasaki disease, nasal polyposis, nephrosis, Netherton's syndrome, pre-eclampsia, prurigo nodularis, pruritus (e.g., chronic pruritus of unknown origin), rhinitis (e.g., allergic rhinitis), rhinosinusitis (e.g., allergic fungal rhinosinusitis, chronic rhinosinusitis with or without nasal polyposis), scleroderma, sickle cell disease, Sjogren's syndrome, tuberculosis, ulcerative colitis, and Whipple's Disease.

In some aspects, the present disclosure provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

A summary of the sequences and the corresponding SEQ ID NOs referenced herein is shown in Table 1, below.

TABLE 1
Informal Sequence Listing

| SEQ | | |
|---|---|---|
| ID | | |
| NO | Sequence | Description |
| 1 | EVQLVESGGGLEQPGGSLRLSCAGSGFTFRDYAMTWVRQAPGKGLE | Dupilumab HCVR |
| | WVSSISGSGGNTYYADSVKGRFTISRDNSKNTLYLQMNSLRAEDTA | amino acid sequence |
| | VYYCAKDRLSITIRPRYYGLDVWGQGTTVTVS | |
| 2 | DIVMTQSPLSLPVTPGEPASISCRSSQSLLYSIGYNYLDWYLQKSGQS | Dupilumab LCVR |
| | PQLLIYLGSNRASGVPDRFSGSGSGTDFTLKISRVEAEDVGFYYCMQ | amino acid sequence |
| | ALQTPYTFGQGTKLEIK | |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 |
| | | amino acid sequence |
| 7 | LGS | Dupilumab LCDR2 |
| | | amino acid sequence |
| 8 | MQALQTPYT | Dupilumab LCDR3 |
| | | amino acid sequence |
| 9 | EVQLVESGGGLEQPGGSLRLSCAGSGFTFRDYAMTWVRQAPGKGLE | Dupilumab heavy chain |
| | WVSSISGSGGNTYYADSVKGRFTISRDNSKNTLYLQMNSLRAEDTA | amino acid sequence |
| | VYYCAKDRLSITIRPRYYGLDVWGQGTTVTVSSASTKGPSVFPLAPC | |
| | SRSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCPP | |
| | CPAPEFLGGPSVFLFPPKPKDTLMISRTPEVTCVVVDVSQEDPEVQFN | |
| | WYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKS | |
| | RWQEGNVFSCSVMHEALHNHYTQKSLSLSLGK | |
| 10 | DIVMTQSPLSLPVTPGEPASISCRSSQSLLYSIGYNYLDWYLQKSGQS | Dupilumab light chain |
| | PQLLIYLGSNRASGVPDRFSGSGSGTDFTLKISRVEAEDVGFYYCMQ | amino acid sequence |
| | ALQTPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKA | |
| | DYEKHKVYACEVTHQGLSSPVTKSFNRGEC | |
| 11 | MKVLQEPTCVSDYMSISTCEWKMNGPTNCSTELRLLYQLVFLLSEA | Human IL-4Rα |
| | HTCIPENNGGAGCVCHLLMDDVVSADNYTLDLWAGQQLLWKGSF | |
| | KPSEHVKPRAPGNLTVHTNVSDTLLLTWSNPYPPDNYLYNHLTYAV | |
| | NIWSENDPADFRIYNVTYLEPSLRIAASTLKSGISYRARVRAWAQCY | |
| | NTTWSEWSPSTKWHNSYREPFEQH | |
| 12 | EIVLTQSPGTLSLSPGERATLSCRASQSVSNSYLAWYQQKPGQAPRL | SCB-VL-39 |
| | LIFGASSRATGIPDRFSGSGSGTDFTLTISRLEPEDFAVYYCQQYGSSP | |
| | PWTFGQGTKVEIK | |
| 13 | EIVLTQSPGTLSLSPGERATLSCRASQSVSSSYLAWYQQKPGQAPRLL | SCB-VL-40 |
| | IYGASSRATGIPDRFSGSGSGTDFTLTISRLEPEDFAVYYCQQYGSSPP | |
| | WTFGQGTKVEIK | |
| 14 | EIVLTQSPGTLSLSPGERATLSCRASQSVSSSYLAWYQQKPGQAPRLL | SCB-VL-41 |
| | IFGASSRAPGIPDRFSGSGSGTDFTLTISRLEPEDFAVYYCQQYGSSPP | |
| | WTFGQGTKVEIK | |
| 15 | EIVLTQSPGTLSLSPGERATLSCRASQSVSNSYLAWYQQKPGQAPRL | SCB-VL-42 |
| | LIYGASSRATGIPDRFSGSGSGTDFTLTISRLEPEDFAVYYCQQYGSSP | |
| | PWTFGQGTKVEIK | |
| 16 | EIVLTQSPGTLSLSPGERATLSCRASQSVSNSYLAWYQQKPGQAPRL | SCB-VL-43 |
| | LIFGASSRAPGIPDRFSGSGSGTDFTLTISRLEPEDFAVYYCQQYGSSP | |
| | PWTFGQGTKVEIK | |
| 17 | EIVLTQSPGTLSLSPGERATLSCRASQSVSSSYLAWYQQKPGQAPRLL | SCB-VL-44 |
| | IYGASSRAPGIPDRFSGSGSGTDFTLTISRLEPEDFAVYYCQQYGSSPP | |
| | WTFGQGTKVEIK | |
| 18 | EIVLTQSPGTLSLSPGERATLSCRASQSVSSSYLAWYQQKPGQAPRLL | SCB-VL-45 |
| | IFGASSRATGIPDRFSGSGSGTDFTLTISRLEPEDFAVYYCQQYDHSPP | |
| | WTFGQGTKVEIK | |
| 19 | EIVLTQSPGTLSLSPGERATLSCRASQSVSSSYLAWYQQKPGQAPRLL | SCB-VL-46 |
| | IFGASSRATGIPDRFSGSGSGTDFTLTISRLEPEDFAVYYCQQYGSSAG | |
| | WTFGQGTKVEIK | |
| 20 | EIVLTQSPGTLSLSPGERATLSCRASQSVSSSYLAWYQQKPGQAPRLL | SCB-VL-47 |
| | IFGASSRATGIPDRFSGSGSGTDFTLTISRLEPEDFAVYYCQQYDHSA | |
| | GWTFGQGTKVEIK | |
| 21 | EIVLTQSPGTLSLSPGERATLSCRASQSVSNSYLAWYQQKPGQAPRL | SCB-VL-48 |
| | LIFGASSRATGIPDRFSGSGSGTDFTLTISRLEPEDFAVYYCQQYDHSP | |
| | PWTFGQGTKVEIK | |
| 22 | EIVLTQSPGTLSLSPGERATLSCRASQSVSSSYLAWYQQKPGQAPRLL | SCB-VL-49 |
| | IYGASSRATGIPDRFSGSGSGTDFTLTISRLEPEDFAVYYCQQYDHSPP | |
| | WTFGQGTKVEIK | |
| 23 | EIVLTQSPGTLSLSPGERATLSCRASQSVSSSYLAWYQQKPGQAPRLL | SCB-VL-50 |
| | IFGASSRAPGIPDRFSGSGSGTDFTLTISRLEPEDFAVYYCQQYDHSPP | |
| | WTFGQGTKVEIK | |
| 24 | EIVLTQSPGTLSLSPGERATLSCRASQSVSSSYLAWYQQKPGQAPRLL | SCB-VL-51 |
| | IYGASSRAPGIPDRFSGSGSGTDFTLTISRLEPEDFAVYYCQQYDHSA | |
| | GWTFGQGTKVEIK | |
| 25 | EIVLTQSPGTLSLSPGERATLSCRASQSVSNSYLAWYQQKPGQAPRL | SCB-VL-52 |
| | LIFGASSRAPGIPDRFSGSGSGTDFTLTISRLEPEDFAVYYCQQYDHSA | |
| | GWTFGQGTKVEIK | |
| 26 | EIVLTQSPGTLSLSPGERATLSCRASQSVSNSYLAWYQQKPGQAPRL | SCB-VL-53 |
| | LIYGASSRATGIPDRFSGSGSGTDFTLTISRLEPEDFAVYYCQQYDHS | |
| | AGWTFGQGTKVEIK | |
| 27 | EIVLTQSPGTLSLSPGERATLSCRASQSVSSSYLAWYQQKPGQAPRLL | SCB-VL-54 |
| | IFGASSRAPGIPDRFSGSGSGTDFTLTISRLEPEDFAVYYCQQYDHSA | |
| | GWTFGQGTKVEIK | |
| 28 | EIVLTQSPGTLSLSPGERATLSCRASQSVSSSYLAWYQQKPGQAPRLL | SCB-VL-55 |
| | IYGASSRATGIPDRFSGSGSGTDFTLTISRLEPEDFAVYYCQQYDHSA | |
| | GWTFGQGTKVEIK | |
| 29 | EIVLTQSPGTLSLSPGERATLSCRASQSVSNSYLAWYQQKPGQAPRL | SCB-VL-56 |
| | LIFGASSRATGIPDRFSGSGSGTDFTLTISRLEPEDFAVYYCQQYDHSA | |
| | GWTFGQGTKVEIK | |
| 30 | EIVLTQSPGTLSLSPGERATLSCRASQSVSSSYLAWYQQKPGQAPRLL | SCB-VL-57 |
| | IFGASSRATGIPDRFSGSGSGTDFTLTISRLEPEDFAVYYCQQYGSSPP | |
| | WTFGQGTKVEIK | |
| 31 | EIVLTQSPGTLSLSPGERATLSCRASQSVSNSYLAWYQQKPGQAPRL | SCB-VL-58 |
| | LIYGASSRAPGIPDRFSGSGSGTDFTLTISRLEPEDFAVYYCQQYDHS | |
| | AGWTFGQGTKVEIK | |
| 32 | EVQLVESGGGLVHPGGSLRLSCAGSGFTFSRNAMFWVRQAPGKGLE | SCB-VH-59 |
| | WVSGIGTGGATNYADSVKGRFTISRDNAKNSLYLQMNSLRAEDMA | |
| | VYYCARGRYYFDYWGQGTLVTVSS | |
| 33 | EVQLVQSGGGLVQPGGSLRLSCAGSGFTFSRNAMFWVRQAPGKGLE | SCB-VH-60 |
| | WVSGIGTGGATNYADSVKGRFTISRDNAKNSLYLQMNSLRAEDMA | |
| | VYYCARGRYYFDYWGQGTLVTVSS | |
| 34 | EVQLVQSGGGLVHPGGSLRLSCAASGFTFSRNAMFWVRQAPGKGLE | SCB-VH-61 |
| | WVSGIGTGGATNYADSVKGRFTISRDNAKNSLYLQMNSLRAEDMA | |
| | VYYCARGRYYFDYWGQGTLVTVSS | |
| 35 | EVQLVQSGGGLVHPGGSLRLSCAGSGFTFSRNAMFWVRQAPGKGLE | SCB-VH-62 |
| | WVSGIGTGGATSYADSVKGRFTISRDNAKNSLYLQMNSLRAEDMA | |
| | VYYCARGRYYFDYWGQGTLVTVSS | |
| 36 | EVQLVQSGGGLVHPGGSLRLSCAGSGFTFSRNAMFWVRQAPGKGLE | SCB-VH-63 |
| | WVSGIGTGGATNYADSVKGRFTISRDNAKNSLYLQMNSLRAEDTAV | |
| | YYCARGRYYFDYWGQGTLVTVSS | |
| 37 | EVQLVESGGGLVQPGGSLRLSCAGSGFTFSRNAMFWVRQAPGKGLE | SCB-VH-64 |
| | WVSGIGTGGATNYADSVKGRFTISRDNAKNSLYLQMNSLRAEDMA | |
| | VYYCARGRYYFDYWGQGTLVTVSS | |
| 38 | EVQLVESGGGLVHPGGSLRLSCAASGFTFSRNAMFWVRQAPGKGLE | SCB-VH-65 |
| | WVSGIGTGGATNYADSVKGRFTISRDNAKNSLYLQMNSLRAEDMA | |
| | VYYCARGRYYFDYWGQGTLVTVSS | |
| 39 | EVQLVQSGGGLVQPGGSLRLSCAASGFTFSRNAMFWVRQAPGKGLE | SCB-VH-66 |
| | WVSGIGTGGATNYADSVKGRFTISRDNAKNSLYLQMNSLRAEDMA | |
| | VYYCARGRYYFDYWGQGTLVTVSS | |
| 40 | EVQLVQSGGGLVHPGGSLRLSCAGSGFTFSRNAMFWVRQAPGKGLE | SCB-VH-67 |
| | WVSGIGTGGATSYADSVKGRFTISRDNAKNSLYLQMNSLRAEDTAV | |
| | YYCARGRYYFDYWGQGTLVTVSS | |
| 41 | EVQLVQSGGGLVHPGGSLRLSCAGSGFTFSRNAMFWVRQAPGKGLE | SCB-VH-68 |
| | WVSGIGTGGATNYADSVKGRFTISRDNAKNSLYLQMNSLRAEDMA | |
| | VYYCARGRYYFPWWGQGTLVTVSS | |
| 42 | EVQLVESGGGLVHPGGSLRLSCAGSGFTFSRNAMFWVRQAPGKGLE | SCB-VH-69 |
| | WVSGIGTGGATNYADSVKGRFTISRDNAKNSLYLQMNSLRAEDMA | |
| | VYYCARGRYYFPWWGQGTLVTVSS | |
| 43 | EVQLVQSGGGLVQPGGSLRLSCAGSGFTFSRNAMFWVRQAPGKGLE | SCB-VH-70 |
| | WVSGIGTGGATNYADSVKGRFTISRDNAKNSLYLQMNSLRAEDMA | |
| | VYYCARGRYYFPWWGQGTLVTVSS | |
| 44 | EVQLVQSGGGLVHPGGSLRLSCAASGFTFSRNAMFWVRQAPGKGLE | SCB-VH-71 |
| | WVSGIGTGGATNYADSVKGRFTISRDNAKNSLYLQMNSLRAEDMA | |
| | VYYCARGRYYFPWWGQGTLVTVSS | |
| 45 | EVQLVQSGGGLVHPGGSLRLSCAGSGFTFSRNAMFWVRQAPGKGLE | SCB-VH-72 |
| | WVSGIGTGGATSYADSVKGRFTISRDNAKNSLYLQMNSLRAEDMA | |
| | VYYCARGRYYFPWWGQGTLVTVSS | |
| 46 | EVQLVQSGGGLVHPGGSLRLSCAGSGFTFSRNAMFWVRQAPGKGLE | SCB-VH-73 |
| | WVSGIGTGGATNYADSVKGRFTISRDNAKNSLYLQMNSLRAEDTAV | |
| | YYCARGRYYFPWWGQGTLVTVSS | |
| 47 | EVQLVQSGGGLVHPGRSLRLSCAGSGFTFSRNAMFWVRQAPGKGLE | SCB-VH-74 |
| | WVSGIGTGGATNYADSVKGRFTISRDNAKNSLYLQMNSLRAEDMA | |
| | VYYCARGRYYFDYWGQGTLVTVSS | |
| 48 | EVQLVQSGGGLVHPGGSLRLTCAGSGFTFSRNAMFWVRQAPGKGL | SCB-VH-75 |
| | EWVSGIGTGGATNYADSVKGRFTISRDNAKNSLYLQMNSLRAEDM | |
| | AVYYCARGRYYFDYWGQGTLVTVSS | |
| 49 | EVQLVQSGGGLVHPGGSLRLSCAGSGFTFSRNAMHWVRQAPGKGL | SCB-VH-76 |
| | EWVSGIGTGGATNYADSVKGRFTISRDNAKNSLYLQMNSLRAEDM | |
| | AVYYCARGRYYFDYWGQGTLVTVSS | |
| 50 | EVQLVQSGGGLVHPGGSLRLSCAGSGFTFSRNAMFWVRQAPGEGLE | SCB-VH-77 |
| | WVSGIGTGGATNYADSVKGRFTISRDNAKNSLYLQMNSLRAEDMA | |
| | VYYCARGRYYFDYWGQGTLVTVSS | |
| 51 | EVQLVQSGGGLVHPGGSLRLSCAGSGFTFSRNAMFWVRQAPGKGLE | SCB-VH-78 |
| | WVSGIGTGGATNYADSVKGRFTISRDEAKNSLYLQMNSLRAEDMA | |
| | VYYCARGRYYFDYWGQGTLVTVSS | |
| 52 | EVQLVQSGGGLVHPGGSLRLSCAGSGFTFSRNAMFWVRQAPGKGLE | SCB-VH-79 |
| | WVSGIGTGGATNYADSVKGRFTISRDNAKNSLYLQMNSLRAGDMA | |
| | VYYCARGRYYFDYWGQGTLVTVSS | |
| 53 | EVQLVQSGGGLVHPGGSLRLSCAGSGFTFDDYAMFWVRQAPGKGL | SCB-VH-80 |
| | EWVSGIGTGGATNYADSVKGRFTISRDNAKNSLYLQMNSLRAEDM | |
| | AVYYCARGRYYFDYWGQGTLVTVSS | |
| 54 | EVQLVQSGGGLVQPGGSLRLSCAASGFTFSRNAMFWVRQAPGKGLE | SCB-VH-81 |
| | WVSGIGTGGATSYADSVKGRFTISRDNAKNSLYLQMNSLRAEDTAV | |
| | YYCARGRYYFPWWGQGTLVTVSS | |
| 55 | EVQLVESGGGLVHPGGSLRLSCAASGFTFSRNAMFWVRQAPGKGLE | SCB-VH-82 |
| | WVSGIGTGGATSYADSVKGRFTISRDNAKNSLYLQMNSLRAEDTAV | |
| | YYCARGRYYFPWWGQGTLVTVSS | |
| 56 | EVQLVESGGGLVQPGGSLRLSCAGSGFTFSRNAMFWVRQAPGKGLE | SCB-VH-83 |
| | WVSGIGTGGATSYADSVKGRFTISRDNAKNSLYLQMNSLRAEDTAV | |
| | YYCARGRYYFPWWGQGTLVTVSS | |
| 57 | EVQLVESGGGLVQPGGSLRLSCAASGFTFSRNAMFWVRQAPGKGLE | SCB-VH-84 |
| | WVSGIGTGGATNYADSVKGRFTISRDNAKNSLYLQMNSLRAEDTAV | |
| | YYCARGRYYFPWWGQGTLVTVSS | |
| 58 | EVQLVESGGGLVQPGGSLRLSCAASGFTFSRNAMFWVRQAPGKGLE | SCB-VH-85 |
| | WVSGIGTGGATSYADSVKGRFTISRDNAKNSLYLQMNSLRAEDMA | |
| | VYYCARGRYYFPWWGQGTLVTVSS | |
| 59 | EVQLVQSGGGLVHPGGSLRLSCAASGFTFSRNAMFWVRQAPGKGLE | SCB-VH-86 |
| | WVSGIGTGGATSYADSVKGRFTISRDNAKNSLYLQMNSLRAEDTAV | |
| | YYCARGRYYFPWWGQGTLVTVSS | |
| 60 | EVQLVQSGGGLVQPGGSLRLSCAGSGFTFSRNAMFWVRQAPGKGLE | SCB-VH-87 |
| | WVSGIGTGGATSYADSVKGRFTISRDNAKNSLYLQMNSLRAEDTAV | |
| | YYCARGRYYFPWWGQGTLVTVSS | |
| 61 | EVQLVESGGGLVHPGGSLRLSCAGSGFTFSRNAMFWVRQAPGKGLE | SCB-VH-88 |
| | WVSGIGTGGATSYADSVKGRFTISRDNAKNSLYLQMNSLRAEDTAV | |
| | YYCARGRYYFPWWGQGTLVTVSS | |
| 62 | EVQLVQSGGGLVHPGGSLRLSCAGSGFTFSRNAMFWVRQAPGKGLE | SCB-VH-89 |
| | WVSGIGTGGATSYADSVKGRFTISRDNAKNSLYLQMNSLRAEDTAV | |
| | YYCARGRYYFPWWGQGTLVTVSS | |
| 63 | EVQLVESGGGLVQPGGSLRLSCAASGFTFSRNAMFWVRQAPGKGLE | SCB-VH-90 |
| | WVSGIGTGGATNYADSVKGRFTISRDNAKNSLYLQMNSLRAEDMA | |
| | VYYCARGRYYFPWWGQGTLVTVSS | |
| 64 | EVQLVESGGGLVQPGGSLRLSCAASGFTFSRNAMFWVRQAPGKGLE | SCB-VH-91 |
| | WVSGIGTGGATSYADSVKGRFTISRDNAKNSLYLQMNSLRAEDTAV | |
| | YYCARGRYYFDYWGQGTLVTVSS | |
| 65 | EVQLVQSGGGLVHPGGSLRLSCAGSGFTFSRNAMFWVRQAPGKGLE | SCB-VH-92 |
| | WVSGIGTGGATNYADSVKGRFTISRDNAKNSLYLQMNSLRAEDMA | |
| | VYYCARGRYYFDYWGQGTLVTVSS | |
| 66 | EVQLVESGGGLVQPGGSLRLSCAASGFTFSRNAMFWVRQAPGKGLE | SCB-VH-93 |
| | WVSGIGTGGATSYADSVKGRFTISRDNAKNSLYLQMNSLRAEDTAV | |
| | YYCARGRYYFPWWGQGTLVTVSS | |
| 67 | QVQLVQSGAEVKKPGASVKVSCKASGYAFTSYYMHWARQAPGQG | MEDI-1-VH |
| | LEWMGIINPSGGSTSYAQKFQGRVTMTRDTSTSTVYMELSSLRSEDT | |
| | AVYYCARGKWWLDYWGKGTLVTVSS | |
| 68 | QSVLTQPPSVSAAPGQKVTISCSGGSSNIGNSYVSWYQQLPGTAPKL | MEDI-1-VL |
| | LIYDNNKRPSGIPDRFSGSKSGTSATLAITGLQTGDEADYYCGTWDT | |
| | SLSANYVFGTGTKLTVL | |
| 69 | QVQLVQSGAEVKKPGASVKVSCKASGYAFTSYYMHWARQAPGQG | MEDI-2-VH |
| | LEWMGIINPSGGSTSYAQKFQGRVTMTRDTSTSTVYMELSSLRSEDT | |
| | AVYYCARGKWWLYNWGKGTLVTVSS | |
| 70 | QSVLTQPPSVSAAPGQKVTISCSGGSSNIGNSYVSWYQQLPGTAPKL | MEDI-2-VL |
| | LIYDNNKRPSGIPDRFSGSKSGTSATLAITGLQTGDEADYYCGTWDT | |
| | SQPPNPLFGTGTKLTVL | |
| 71 | QVQLVQSGAEVKKPGASVKVSCKASGYAFTSYYMHWARQAPGQG | MEDI-3-VH |
| | LEWMGIINPSGGSTSYAQKFQGRVTMTRDTSTSTVYMELSSLRSEDT | |
| | AVYYCARGKLLKNPWGKGTLVTVSS | |
| 72 | QSVLTQPPSVSAAPGQKVTISCSGGSSNIGNSYVSWYQQLPGTAPKL | MEDI-3-VL |
| | LIYDNNKRPSGIPDRFSGSKSGTSATLAITGLQTGDEADYYCGTWFG | |
| | TPASNYVFGTGTKLTVL | |
| 73 | QVQLVQSGAEVKKPGASVKVSCKASGYAFTSYYMHWARQAPGQG | MEDI-4-VH |
| | LEWMGIINPSGGSTSYAQKFQGRVTMTRDTSTSTVYMELSSLRSEDT | |
| | AVYYCARGKWWLYNWGKGTLVTVSS | |
| 74 | QSVLTQPPSVSAAPGQKVTISCSGGSSNIGNSYVSWYQQLPGTAPKL | MEDI-4-VL |
| | LIYDNNKRPSGIPDRFSGSKSGTSATLAITGLQTGDEADYYCGTWDT | |
| | SSPPQPIFGTGTKLTVL | |
| 75 | QVQLVQSGAEVKKPGASVKVSCKASGYAFTSYYMHWARQAPGQG | MEDI-5-VH |
| | LEWMGIINPSGGSTSYAQKFQGRVTMTRDTSTSTVYMELSSLRSEDT | |
| | AVYYCARGKWWLYDWGKGTLVTVSS | |
| 76 | QSVLTQPPSVSAAPGQKVTISCSGGSSNIGNSYVSWYQQLPGTAPKL | MEDI-5-VL |
| | LIYDNNKRPSGIPDRFSGSKSGTSATLAITGLQTGDEADYYCGTWDT | |
| | SSPPQPIFGTGTKLTVL | |
| 77 | QVQLVQSGAEVKKPGASVKVSCKASGYAFTSYYMHWARQAPGQG | MEDI-6-VH |
| | LEWMGIINPSGGSTSYAQKFQGRVTMTRDTSTSTVYMELSSLRSEDT | |
| | AVYYCARGKYWMYDWGKGTLVTVSS | |
| 78 | QSVLTQPPSVSAAPGQKVTISCSGGSSNIGNSYVSWYQQLPGTAPKL | MEDI-6-VL |
| | LIYDNNKRPSGIPDRFSGSKSGTSATLAITGLQTGDEADYYCGTWDT | |
| | STTYHPIFGTGTKLTVL | |
| 79 | QVQLVQSGAEVKKPGASVKVSCKASGYAFTSYYMHWARQAPGQG | MEDI-7-VH |
| | LEWMGIINPSGGSTSYAQKFQGRVTMTRDTSTSTVYMELSSLRSEDT | |
| | AVYYCARGKWWWQYWGKGTLVTVSS | |
| 80 | QSVLTQPPSVSAAPGQKVTISCSGGSSNIGNSYVSWYQQLPGTAPKL | MEDI-7-VL |
| | LIYDNNKRPSGIPDRFSGSKSGTSATLAITGLQTGDEADYYCGTWDT | |
| | SSPPQPIFGTGTKLTVL | |
| 81 | QVQLVQSGAEVKKPGASVKVSCKASGYAFTSYYMHWARQAPGQG | MEDI-8-VH |
| | LEWMGIINPSGGSTSYAQKFQGRVTMTRDTSTSTVYMELSSLRSEDT | |
| | AVYYCARGKWWWQYWGKGTLVTVSS | |
| 82 | QSVLTQPPSVSAAPGQKVTISCSGGSSNIGNSYVSWYQQLPGTAPKL | MEDI-8-VL |
| | LIYDNNKRPSGIPDRFSGSKSGTSATLAITGLQTGDEADYYCGTWDT | |
| | STTYHPIFGTGTKLTVL | |
| 83 | QVQLVQSGAEVKKPGASVKVSCKASGYAFTSYYMHWARQAPGQG | MEDI-9-VH |
| | LEWMGIINPSGGSTSYAQKFQGRVTMTRDTSTSTVYMELSSLRSEDT | |
| | AVYYCARGKWWLYNWGKGTLVTVSS | |
| 84 | QSVLTQPPSVSAAPGQKVTISCSGGSSNIGNSYVSWYQQLPGTAPKL | MEDI-9-VL |
| | LIYDNNKRPSGIPDRFSGSKSGTSATLAITGLQTGDEADYYCGTWDT | |
| | STTMYPLFGTGTKLTVL | |
| 85 | QVQLVQSGAEVKKPGASVKVSCKASGYAFTSYYMHWARQAPGQG | MEDI-10-VH |
| | LEWMGIINPSGGSTSYAQKFQGRVTMTRDTSTSTVYMELSSLRSEDT | |
| | AVYYCARGKWWLYDWGKGTLVTVSS | |
| 86 | QSVLTQPPSVSAAPGQKVTISCSGGSSNIGNSYVSWYQQLPGTAPKL | MEDI-10-VL |
| | LIYDNNKRPSGIPDRFSGSKSGTSATLAITGLQTGDEADYYCGTWDT | |
| | STVLTPIFGTGTKLTVL | |
| 87 | QVQLVQSGAEVKKPGASVKVSCKASGYAFTSYYMHWARQAPGQG | MEDI-11-VH |
| | LEWMGIINPSGGSTSYAQKFQGRVTMTRDTSTSTVYMELSSLRSEDT | |
| | AVYYCARGKWWFYDWGKGTLVTVSS | |
| 88 | QSVLTQPPSVSAAPGQKVTISCSGGSSNIGNSYVSWYQQLPGTAPKL | MEDI-11-VL |
| | LIYDNNKRPSGIPDRFSGSKSGTSATLAITGLQTGDEADYYCGTWDT | |
| | SPSMIPLFGTGTKLTVL | |
| 89 | QVQLVQSGAEVKKPGASVKVSCKASGYAFTSYYMHWARQAPGQG | MEDI-12-VH |
| | LEWMGIINPSGGSTSYAQKFQGRVTMTRDTSTSTVYMELSSLRSEDT | |
| | AVYYCARGKWWFYDWGKGTLVTVSS | |
| 90 | QSVLTQPPSVSAAPGQKVTISCSGGSSNIGNSYVSWYQQLPGTAPKL | MEDI-12-VL |
| | LIYDNNKRPSGIPDRFSGSKSGTSATLAITGLQTGDEADYYCGTWDT | |
| | STTMYPLFGTGTKLTVL | |
| 91 | QVQLVQSGAEVKKPGASVKVSCKASGYAFTSYYMHWARQAPGQG | MEDI-13-VH |
| | LEWMGIINPSGGSTSYAQKFQGRVTMTRDTSTSTVYMELSSLRSEDT | |
| | AVYYCARGKWWLYDWGKGTLVTVSS | |
| 92 | QSVLTQPPSVSAAPGQKVTISCSGGSSNIGNSYVSWYQQLPGTAPKL | MEDI-13-VL |
| | LIYDNNKRPSGIPDRFSGSKSGTSATLAITGLQTGDEADYYCGTWDT | |
| | STTLQPLFGTGTKLTVL | |
| 93 | QVQLVQSGAEVKKPGASVKVSCKASGYAFTSYYMHWARQAPGQG | MEDI-14-VH |
| | LEWMGIINPSGGSTSYAQKFQGRVTMTRDTSTSTVYMELSSLRSEDT | |
| | AVYYCARGKWWLYNWGKGTLVTVSS | |
| 94 | QSVLTQPPSVSAAPGQKVTISCSGGSSNIGNSYVSWYQQLPGTAPKL | MEDI-14-VL |
| | LIYDNNKRPSGIPDRFSGSKSGTSATLAITGLQTGDEADYYCGTWDT | |
| | SPPTKPLFGTGTKLTVL | |
| 95 | QVQLVQSGAEVKKPGASVKVSCKASGYAFTSYYMHWARQAPGQG | MEDI-15-VH |
| | LEWMGIINPSGGSTSYAQKFQGRVTMTRDTSTSTVYMELSSLRSEDT | |
| | AVYYCARGKWWLYNWGKGTLVTVSS | |
| 96 | QSVLTQPPSVSAAPGQKVTISCSGGSSNIGNSYVSWYQQLPGTAPKL | MEDI-15-VL |
| | LIYDNNKRPSGIPDRFSGSKSGTSATLAITGLQTGDEADYYCGTWDT | |
| | STHRHPLFGTGTKLTVL | |
| 97 | QVQLVQSGAEVKKPGASVKVSCKASGYAFTSYYMHWARQAPGQG | MEDI-16-VH |
| | LEWMGIINPSGGSTSYAQKFQGRVTMTRDTSTSTVYMELSSLRSEDT | |
| | AVYYCARGKWWLYNWGKGTLVTVSS | |
| 98 | QSVLTQPPSVSAAPGQKVTISCSGGSSNIGNSYVSWYQQLPGTAPKL | MEDI-16-VL |
| | LIYDNNKRPSGIPDRFSGSKSGTSATLAITGLQTGDEADYYCGTWDT | |
| | STTYHPIFGTGTKLTVL | |
| 99 | QVQLVQSGAEVKKPGASVKVSCKASGYAFTSYYMHWARQAPGQG | MEDI-17-VH |
| | LEWMGIINPSGGSTSYAQKFQGRVTMTRDTSTSTVYMELSSLRSEDT | |
| | AVYYCARGKWWWQHWGKGTLVTVSS | |
| 100 | QSVLTQPPSVSAAPGQKVTISCSGGSSNIGNSYVSWYQQLPGTAPKL | MEDI-17-VL |
| | LIYDNNKRPSGIPDRFSGSKSGTSATLAITGLQTGDEADYYCGTWDT | |
| | SPVDRPIFGTGTKLTVL | |
| 101 | QVQLVQSGAEVKKPGASVKVSCKASGYAFTSYYMHWARQAPGQG | MEDI-18-VH |
| | LEWMGIINPSGGSTSYAQKFQGRVTMTRDTSTSTVYMELSSLRSEDT | |
| | AVYYCARGKWWWQHWGKGTLVTVSS | |
| 102 | QSVLTQPPSVSAAPGQKVTISCSGGSSNIGNSYVSWYQQLPGTAPKL | MEDI-18-VL |
| | LIYDNNKRPSGIPDRFSGSKSGTSATLAITGLQTGDEADYYCGTWDT | |
| | STTPMPVFGTGTKLTVL | |
| 103 | QVQLVQSGAEVKKPGASVKVSCKASGYAFTSYYMHWARQAPGQG | MEDI-19-VH |
| | LEWMGIINPSGGSTSYAQKFQGRVTMTRDTSTSTVYMELSSLRSEDT | |
| | AVYYCARGKWWWQHWGKGTLVTVSS | |
| 104 | QSVLTQPPSVSAAPGQKVTISCSGGSSNIGNSYVSWYQQLPGTAPKL | MEDI-19-VL |
| | LIYDNNKRPSGIPDRFSGSKSGTSATLAITGLQTGDEADYYCGTWDT | |
| | STTYHPIFGTGTKLTVL | |
| 105 | QVQLVQSGAEVKKPGASVKVSCKASGYAFTSYYMHWARQAPGQG | MEDI-20-VH |
| | LEWMGIINPSGGSTSYAQKFQGRVTMTRDTSTSTVYMELSSLRSEDT | |
| | AVYYCARGKYWMYDWGKGTLVTVSS | |
| 106 | QSVLTQPPSVSAAPGQKVTISCSGGSSNIGNSYVSWYQQLPGTAPKL | MEDI-20-VL |
| | LIYDNNKRPSGIPDRFSGSKSGTSATLAITGLQTGDEADYYCGTWDT | |
| | STVWEWPFGTGTKLTVL | |
| 107 | QVQLVQSGAEVKKPGASVKVSCKASGYAFTSYYMHWARQAPGQG | MEDI-21-VH |
| | LEWMGIINPSGGSASYAQKFQGRVTMTRDTSTSTVYMELSSLRSEDT | |
| | AVYYCARGKYWMYDWGKGTLVTVSS | |
| 108 | QSVLTQPPSVSAAPGQKVTISCSGGSSNIGNSYVSWYQQLPGTAPKL | MEDI-21-VL |
| | LIYDNNKRPSGIPDRFSGSKSGTSATLAITGLQTGDEAVYFCGTWDTS | |
| | TVWEWPFGTGTKLTVL | |
| 109 | QVQLVQSGAEVKKPGASVKVSCKASGYAFTSYYMHWARQAPGQG | MEDI-22-VH |
| | LEWMGIINPSGGSTSYAQKFQGRVTMTRDTSTSTVYMELSSLRSEDT | |
| | AVYYCARGKYWMYDWGKGTLVTVSS | |
| 110 | QPVLTQPPSVSAAPGQKVTISCSGGSSNIGNSYVSWYQQLPGTAPKL | MEDI-22-VL |
| | LIYDNNKRPSGIPDRFSGSKSGTSATLAITGLQTGDEADYFCGTWDTS | |
| | TVWEWPFGTGTKLTVL | |
| 111 | QVQLVQSGAEVRKPGASVKVSCKASGYAFTSYYMHWARQAPGQG | MEDI-23-VH |
| | LEWMGIINPSGGSTSYAQKFQGRVTMTRDTSTSTVYMELSSLRSEDT | |
| | AVYYCARGKYWMYDWGKGTLVTVSS | |
| 112 | QSVLTQPPSVSAAPGQKVTISCSGGSSNIGNNYVSWYQQLPGTAPKL | MEDI-23-VL |
| | LIYDNNKRPPGIPDRFSGSKSGTSATLAITGLQTGDEADYYCGTWDT | |
| | STVWEWPFGTGTKLTVL | |
| 113 | QVQLVQSGAEVKKPGASVKVSCKASGYAFTSYYMHWARQAPGQG | MEDI-24-VH |
| | LEWMGIINPRGGSTSYAQKFQGRVTMTRDTSTSTVYMELSSLRSEDT | |
| | AVYYCARGKYWMYDWGKGTLVTVSS | |
| 114 | QSVLTQPPSVSAAPGQKVTISCSGGSSNIGNSYVSWYQQLPGTAPKL | MEDI-24-VL |
| | LIYDNNKRPSGIPDRFSGSKSGTSATLAITGLQTGDEADYFCGTWDTS | |
| | TVWEWPFGTGTKLTVL | |
| 115 | QVQLVQSGAEVKKPGASVKVSCKASGYAFTSYYMHWARQAPGQG | MEDI-25-VH |
| | LEWMGIINPRGGSASYAQKFQGRVSMTRDTSTSTVYMELSSLRSEDT | |
| | AVYYCARGKYWMYDWGKGTLVTVSS | |
| 116 | QSVLTQPPSVSAAPGQKVTISCSGGSSNIGNSYVSWYQQLPGTAPKL | MEDI-25-VL |
| | LIYDNNKRPSGIPDRFSGSKSGTTATLAITGLQTGDEADYYCGTWVT | |
| | STVWEWPFGTGTKLTVL | |
| 117 | QVQLVQSGAEVKKPGASVKVSCKASGYAFTSYYMHWARQAPGQG | MEDI-26-VH |
| | LEWMGIINPSGGSTSYAQKFQGRVTMTRDTSTSTVYMELSSLRSEDT | |
| | AVYYCARGKYWMYDWGKGTLVTVSS | |
| 118 | QSVLTQPPSVSAAPGQKVTISCSGGSSNIGNSYVSWYQQLPGTAPKL | MEDI-26-VL |
| | LIYDNNKRPSGIPDRFSGSKSGTSATLAITGLQTGDEADYFCGTWDTS | |
| | TVWEWPFGTGTKLTVL | |
| 119 | QVQLVQSGAEVRKPGASVKVSCKASGYAFTSYYMHWARQAPGQG | MEDI-27-VH |
| | LEWMGIINPSGGSTSYAQKFQGRVTMTRDTSTSTVYMELSSLRPEDT | |
| | AVYYCARGKYWMYDWGKGTQVTVSS | |
| 120 | QSVLTQPPLVSAAPGQKVTISCSGGSSNIGNSYVSWYQRLPGTAPKL | MEDI-27-VL |
| | LIYDNNKRPSGIPDRFSGSKSGTSATLAITGLQTGDEADYYCGTWDT | |
| | STVWEWPFGTGTKLTVL | |
| 121 | QVQLVQSGAEVKKPGASVKVSCKASGYAFTSYYMHWARQAPGQG | MEDI-28-VH |
| | LEWMGIINPSGGSTSYAQKFQGRVTMTRDTSTSTVYMELSSLRSEDT | |
| | AVYYCARGKYWMYDWGNGTLVTVSS | |
| 122 | LPVLTQPPSVSAAPGQKVTISCSGGSSSIGNSYVSWYQQLPGAAPKLL | MEDI-28-VL |
| | IYDNNKRPSGIPDRFSGFRSGTSATLAITGLQTGDEADYYCGTWDTSP | |
| | VWEWPFGTGTKLTVL | |
| 123 | QVQLVQSGAEVKKPGASVKVSCKASGYAFTSYYMHWARQAPGQG | MEDI-29-VH |
| | LEWMGIINPSGGSTSYAQKFQGRVTMTRDTSTSTVYMELSSLRSEDT | |
| | AVYYCARGKYWMYDWGKGTRVTVSS | |
| 124 | QSVLTQPPSVSAAPGQKVTISCSGGSSNIGNSYVSWYQQLPGTAPKL | MEDI-29-VL |
| | LIYDNNKRPSGIPDRFSGSKSGTSATLAITGLQTGDEADYYCGTWDT | |
| | SPVWEWPFGTGTKLTVL | |
| 125 | QVQLVQSGAEVKKPGASVKVSCKASGYAFTSYYMHWARQAPGQG | MEDI-30-VH |
| | LEWMGIINPSGGSTSYAQKFQGRVTMTRDTSTSTVYMELSSLRSEDT | |
| | AVYYCARGKYWMYDWGKGTLVTVSS | |
| 126 | QSVLTQPPSVSAAPGQKVTISCSGGSSNIGNSYVSWYQRLPGAAPKL | MEDI-30-VL |
| | LIYDNNKRPSGIPDRFSGSKSGTSATLAITGLQTGDEADYYCGTWDT | |
| | STVWEWPFGTGTKLTVL | |
| 127 | QVQLVQSGAEVKKPGASVKVSCKASGYAFTSYYMHWARQAPGQG | MEDI-31-VH |
| | LEWMGIINPSGGSTSYAQKFQGRVTMTRDTSTSTVYMELSSLRSEDT | |
| | AVYYCARGKYWMYDWGKGTLVTVSS | |
| 128 | QSVLTQPPSVSAAPGQKVTISCSGGSSSIGNSYVSWYQQLPGTAPKLL | MEDI-31-VL |
| | IYDNNKRPSGIPDRFSGSKSGTSATLAITGLQTGDEADYYCGTWATS | |
| | PVWEWPFGTGTKLTVL | |
| 129 | QVQLVQSGAEVKKPGASVKVSCKASGYAFTSYYMHWARQAPGQG | MEDI-32-VH |
| | LEWMGIINPSGGSTSYAQKFQGRVTMTRDTSTSTVYMELSSLRSEDT | |
| | AVYYCARGKYWMYDWGKGTLVTVSS | |
| 130 | QSVLTQPPSVSAAPGQKVTISCSGGSSNIGNSYVSWYQQLPGTAPKL | MEDI-32-VL |
| | LIYDNNKRPSGIPDRFSGSKSGTSATLAITGLQTGDEADYFCGTWDTS | |
| | TAWEWPFGTGTKLTVL | |
| 131 | QVQLVQSGAEEKKPGASVKVSCKASGYAFTSYYMHWARQAPGQG | MEDI-33-VH |
| | LEWMGIINPSGGSTSYAQKFQGRVTMTRDTSTSTVYMELSSLRSEDT | |
| | AVYYCARGKYWMYDWGKGTLVTVSS | |
| 132 | QSALTQPPSVSAAPGQKVTISCSGGSSNIGNSYVSWYQQLPGTAPKL | MEDI-33-VL |
| | LIYDNNKRPSGIPDRFSGSKSGTSATLAITGLQTGDEADYFCGTWDTS | |
| | TVWEWPFGTGTKLTVL | |
| 133 | QVQLVQSGAEVKKPGASVKVSCKASGYAFTSYYMHWARQAPGQG | MEDI-34-VH |
| | LEWMGIINPSGGSTSYAQKFQGRVSMTRDTSTSTVYMELSSLRSEDT | |
| | AVYYCARGKYWMYDWGKGTLVTVSS | |
| 134 | QSVLTQPPSVSAAPGQKVTISCSGGSSNIGNSYVSWYQQLPGTAPKL | MEDI-34-VL |
| | LIYDNNKRPSGIPDRFSGSKSGTSATLAITGLQTGDEADYFCGTWDTS | |
| | TVWEWPFGTGTKLTVL | |
| 135 | QVQLVQSGAEVKKPGASVKVSCKASGYAFTSYYMHWARQAPGQG | MEDI-35-VH |
| | LEWMGIINPSGGSTSYAQKFQGRVTMTRDTSTSTVYMELSSLRSEDT | |
| | AVYYCARGKYWMYDWGKGTLVTVSS | |
| 136 | QSVLTQPPSVSAAPGQKVTISCSGGSSNIGNSYVSWYQQLPGTAPKL | MEDI-35-VL |
| | LIYDNNKRPSGIPDRFSGSKSGTSATLAITGLQTGDEADYYCGTWDT | |
| | SPVWEWPFGTGTKLTVL | |
| 137 | QVQLVQSGAEVKKPGASVKVSCKASGYAFTSYYMHWARQAPGQG | MEDI-36-VH |
| | LEWMGIINPSGGSASYAQKFQGRVTMTRDTSTSTVYMELSSLRSEDT | |
| | AVYYCARGKYWMYDWGKGTLVTVSS | |
| 138 | QSVLTQPPSVSAAPGQKVTISCSGGSSNIGNSYVSWYQQLPGTAPKL | MEDI-36-VL |
| | LIYDNNKRPSGIPDRFSGSKSGTSATLAITGLQTGDEADYYCGTWDS | |
| | STVWEWPFGTGTKLTVL | |
| 139 | QVQLVQSGAEVKKPGASVKVSCKASGYAFTSYYMHWARQAPGQG | MEDI-37-VH |
| | LEWMGIINPRGGSTSYAQKFQGRVAMTRDTSTSTVYMELSSLRPEDT | |
| | AVYYCARGKYWMYDWGKGTLVTVSS | |
| 140 | QSVLTQPPSVSAAPGQKVTISCSGGGSSIGNSYVSWYQQLPGTAPKL | MEDI-37-VL |
| | LIYDNNKRPSGVPDRFSGSKSGTSATLAITGLQTGDEADYYCGTWDT | |
| | SPVWEWPFGTGTKLTVL | |
| 141 | QVQLVQSGAEVKKPGASVKVSCKASGYAFTSYYMHWARQAPGQG | MEDI-38-VH |
| | LEWMGIINPSGGSASYAQKFQGRVTMTRDTSTSTVYMELSSLRSEDT | |
| | AVYYCARGKYWMYDWGKGTLVTVSS | |
| 142 | QSVLTQPPSVSAAPGQKVTISCSGGSSNIGNSYVSWYQQLPGTAPKL | MEDI-38-VL |
| | LIYDNNKRPSGIPDRFSGSKSGTSATLAITGLQTGDEADYFCGTWDTS | |
| | TVWEWPFGTGTKLTVL | |
| 143 | QVQLVQSGAEVKKPGASVKVSCKASGYAFTSYYMHWARQAPGQG | MEDI-39-VH |
| | LEWMGIINPRGGSTSYAQKFQGRVTMTRDTSTSTVYMELSSLRSEDT | |
| | AVYYCARGKYWMYDWGKGTLVTVSS | |
| 144 | QSVLTQPPSVSAAPGQKVTISCSGGSSNIGNSYVSWYQQLPGTAPKL | MEDI-39-VL |
| | LIYDNNKRPSGIPDRFSGSKSGTSATLAITGLQTGDEADYYCGTWDT | |
| | STAWEWPFGTGTKLTVL | |
| 145 | QVQLVQSGAEVKKPGASVKVSCKASGYAFTSYYMHWARQAPGQG | MEDI-40-VH |
| | LEWMGIINPSGGSTSYAQKFQGRVTMTRDTSTSTVYMELSSLRSEDT | |
| | AVYYCARGKYWMYDWGKGTLVTVSS | |
| 146 | QSVLTQPPSVSAAPGQKVTISCSGGSSNIGNSYVSWYQQLPGTAPKL | MEDI-40-VL |
| | LIYDNNKRPSGIPDRFSGSKSGTSATLAITGLQTGDEADYYCGTWDS | |
| | STVWEWPFGTGTKLTVL | |
| 147 | QVQLVQSGAEVRKPGASVKVSCKASGYAFTSYYMHWARQAPGQG | MEDI-41-VH |
| | LEWMGIINPSGGSTSYAQKFQGRVTMTRDTSTSTVYMELSSLRPEDT | |
| | AVYYCARGKYWMYDWGKGTLVTVSG | |
| 148 | QSVLTQPPSVSAAPGQKVTISCSGGSTNIGNSYVSWYQRLPGTAPKL | MEDI-41-VL |
| | LIYDNNKRPPGIPDRFSGSKSGTSATLAITGLQTGDEADYYCGTWDT | |
| | STVWEWPFGTGTKLTVL | |
| 149 | QVQLVQSGAEVKKPGASVKVSCKASGYAFTSYYMHWARQAPGQG | MEDI-42-VH |
| | LEWVGIINPSGGSTSYAQKFQGRVTMTRDTSTSTVYMELSSLRSGDT | |
| | AVYYCARGKYWMYDWGKGTLVTVSS | |
| 150 | QAVLTQPPSVSAAPGQKVTISCSGGSSNIGNSYVSWYQRLPGAAPKL | MEDI-42-VL |
| | LIYDNNKRPSGIPDRFSGSKSGTSATLAITGLQTGDEADYYCGTWDT | |
| | STGWEWPFGTGTKLTVL | |
| 151 | QVQLVQSGAEVKKPGASVKVSCKASGYAFTSYYMHWVRQAPGQG | MEDI-37GL-VH |
| | LEWMGIINPRGGSTSYAQKFQGRVTMTRDTSTSTVYMELSSLRSEDT | |
| | AVYYCARGKYWMYDWGKGTLVTVSS | |
| 152 | QSVLTQPPSVSAAPGQKVTISCSGGGSSIGNSYVSWYQQLPGTAPKL | MEDI-37GL-VL |
| | LIYDNNKRPSGIPDRFSGSKSGTSATLGITGLQTGDEADYYCGTWDT | |
| | SPVWEWPFGTGTKLTVL | |
| 153 | EVQLLESGGGLVQPGGSLRLSCAVSGFTFSNYAMSWVRQAPGKGLE | AJOU-1-VH |
| | WVSAISSGGGNIYYADSVKGRFTISRDNSKNTLYLQMNSLRAEDTA | |
| | VYYCAKLRRYFDYWGQGTLVTVSS | |
| 154 | EVQLLESGGGLVQPGGSLRLSCAASGFTFSDYAMSWVRQAPGKGLE | AJOU-2-VH |
| | WVSAISSGGSSIYYADSVKGRFTISRDNSKNTLHLQMNSLRAEDTAV | |
| | YYCARGPQRSATAVFDYWGQGTLVTVSS | |
| 155 | EVQLLESGGGLVQPGGSLRLSCAASGFTFSNYAMSWVRQAPGKGLE | AJOU-3-VH |
| | WVSWISPNSGNIYYADSVKGRFTISRDNSKNTLYLQMNSLRAEDTA | |
| | VYYCARRPLSAAWSHSSYYNAMDVWGQGTLVTVSS | |
| 156 | EVQLLESGGGLVQPGGSLRLSCAASGFTFSGYAMSWVRQAPGKGLE | AJOU-4-VH |
| | WVSLISHSGSNTYYADSVKGRFTISRDNSKNTLYLQMNSLRAEDTA | |
| | VYYCARPHRAFDYWGQGTLVTVSS | |
| 157 | EVQLLESGGGLVQPGGSLRLSCAASGFTFSNYAMSWVRQAPGKGLE | AJOU-5-VH |
| | WVSGISHGSGSIYYADSVKGRFTISRDNSKNTLYLQMNSLRAEDTAV | |
| | YYCARPHRAFDYWGQGTLVTVSS | |
| 158 | EVQLLESGGGLVQPGGSLRLSCAASGFTFSNYAMSWVRQAPGKGLE | AJOU-6-VH |
| | WVSGISHGNGSIYYADSVKGRFTISRDNSKNTLYLQMNSLRAEDTA | |
| | VYYCAKTGRHFDYWGQGTLVTVSS | |
| 159 | EVQLLESGGGLVQPGGSLRLSCAASGFTFSNYAMSWVRQAPGKGLE | AJOU-7-VH |
| | WVSSISPSGSSIYYADSVKGRFTISRDNSKNTLYLQMNSLRAEDTAV | |
| | YYCARSYRAFDYWGQGTLVTVSS | |
| 160 | EVQLLESGGGLVQPGGSLRLSCAASGFTFSNYAMSWVRQAPGKGLE | AJOU-8-VH |
| | WVSAISPSGGSIYYADSVKGRFTISRDNSKNTLYLQMNSLRAEDTAV | |
| | YYCARAKRAFDYWGQGTLVTVSS | |
| 161 | EVQLLESGGGLVQPGGSLRLSCAASGFTFSNYAMSWVRQAPGKGLE | AJOU-9-VH |
| | WVSAISPGSGSTYYADSVKGRFTISRDNSKNTLYLQMNSLRAEDTAV | |
| | YYCAKFRRHFDYWGQGTLVTVSS | |
| 162 | EVQLLESGGGLVQPGGSLRLSCAASGFTFSNYAMSWVRQAPGKGLE | AJOU-10-VH |
| | WVSAISSGGGNIYYADSVKGRFTISRDNSKNTLYLQMNSLRAEDTA | |
| | VYYCARVHRAFDYWGQGTLVTVSS | |
| 163 | EVQLLESGGGLVQPGGSLRLSCAASGFTFSNYAMSWVRQAPGKGLE | AJOU-69-VH |
| | WVSAITSSGRSIYYADSVKGRFTISRDNSKNTLYLQMNSLRAEDTAV | |
| | YYCARVHRAFDYWGQGTLVTVSS | |
| 164 | EVQLLESGGGLVQPGGSLRLSCAASGFTFSNYAMSWVRQAPGKGLE | AJOU-70-VH |
| | WVSAITSSGANIYYADSVKGRFTISRDNSKNTLYLQMNSLRAEDTAV | |
| | YYCARVHRAFDYWGQGTLVTVSS | |
| 165 | EVQLLESGGGLVQPGGSLRLSCAASGFTFSNYAMSWVRQAPGKGLE | AJOU-71-VH |
| | WVSAITSSGGNIYYADSVKGRFTISRDNSKNTLYLQMNSLRAEDTAV | |
| | YYCARVHRAFDYWGQGTLVTVSS | |
| 166 | EVQLLESGGGLVQPGGSLRLSCAASGFTFSNYAMSWVRQAPGKGLE | AJOU-72-VH |
| | WVSAITAGGGSIYYADSVKGRFTISRDNSKNTLYLQMNSLRAEDTA | |
| | VYYCARVHRAFDYWGQGTLVTVSS | |
| 167 | EVQLLESGGGLVQPGGSLRLSCAASGFTFSRHAMAWVRQAPGKGLE | AJOU-83-VH |
| | WVSAITSSGRSIYYADSVKGRFTISRDNSKNTLYLQMNSLRAEDTAV | |
| | YYCARVHRAFDYWGQGTLVTVSS | |
| 168 | QSVLTQPPSASGTPGQRVTISCSGSSSNIGNNYVNWYQQLPGTAPKL | AJOU-33-VL |
| | LIYDNSHRPSGVPDRFSGSKSGTSASLAISGLRSEDEADYYCGTWDA | |
| | SLSAYVFGGGTKLTVL | |
| 169 | QSVLTQPPSASGTPGQRVTISCSGSSSNIGNNNVSWYQQLPGTAPKLL | AJOU-34-VL |
| | IYANSKRPSGVPDRFSGSKSGTSASLAISGLRSEDEADYYCGSWDDS | |
| | LSAYVFGGGTKLTVL | |
| 170 | QSVLTQPPSAPGTPGQRVTISCTGSSSNIGSNSVNWYQQLPGTAPKLL | AJOU-35-VL |
| | IYDDSHRPSGVPDRFSGSKSGTSASLAISGLRSEDEADYYCDAWDSS | |
| | LSAYVFGGGTKLTVL | |
| 171 | QSVLTQPPSASGTPGQRVTLSCTGSSSNIGSNYVSWYQQLPGTAPKL | AJOU-36-VL |
| | LIYADSQRPSGVPDRFSGSKSGTSASLAISGLRSEDEADYYCGTWDD | |
| | SLSGYVFGGGTKLTVL | |
| 172 | QSVLTQPPSASGTPGQRVTISCSSSSSNIGSNYVSWYQQLPGTAPKLLI | AJOU-37-VL |
| | YSDSHRPSGVPDRFSGSKSGTSASLAISGLRSEDEADYYCGSWDYSL | |
| | SAYVFGGGTKLTVL | |
| 173 | QSVLTQPPSASGTPGQRVTISCTGSSSNIGNNTVSWYQQLPGTAPKLL | AJOU-38-VL |
| | IYDNSHRPSGVPDRFSGSKSGTSASLAISGLQSEDEADYYCGSWDYS | |
| | LSAYVFGGGTKLTVL | |
| 174 | QSVLTQPPSASGTPGQRVTISCTGSSSNIGNNDVNWYQQLPGTAPKL | AJOU-39-VL |
| | LIYYDSQRPSGVPDRFSGSKSGTSASLAISGLRSEDEADYYCATWDA | |
| | SLSAYVFGGGTKLTVL | |
| 175 | QSVLTQPPSASGTPGQRVTISCSGSSSNIGSNAVNWYQQLPGTAPKLL | AJOU-40-VL |
| | IYYDNQRPSGVPDRFSGSKSGTSASLAISGLRSEDEADYYCGTWDDS | |
| | LNGYVFGGGTKLTVL | |
| 176 | QSVLTQPPSASGTPGQRVTISCSGSSSNIGNNAVTWYQQLPGTAPKLL | AJOU-41-VL |
| | IYDDSHRPSGVPDRFSGSKSGTSASLAISGLRSEDEADYYCGSWDYS | |
| | LSAYVFGGGTKLTVL | |
| 177 | QSVLTQPPSASGTPGQRVTISCSGSSSNIGSNTFNWYQQLPGTAPKLLI | AJOU-42-VL |
| | YADSHRPSGVPDRFSGSKSGTSASLAISGLRSEDEADYYCGTWDYSL | |
| | SGYVLGGGTKLTVL | |
| 178 | QSVLTQPPSASGTPGQRVTISCSGSSSNIGSNTFNWYQQLPGTAPKLLI | AJOU-77-VL |
| | YADSHRPSGVPDRFSGSKSGTSASLAISGLRSEDEADYYCGTWDYSL | |
| | SGYVLGGGTKLTVL | |
| 179 | QSVLTQPPSASGTPGQRVTISCSGSSSNIGSNTFNWYQQLPGTAPKLLI | AJOU-78-VL |
| | YADSHRPSGVPDRFSGSKSGTSASLAISGLRSEDEADYYCGTWDYSL | |
| | RGYVLGGGTKLTVL | |
| 180 | QSVLTQPPSASGTPGQRVTISCSGSSSNIGSNTFNWYQQLPGTAPKLLI | AJOU-79-VL |
| | YADSHRPSGVPDRFSGSKSGTSASLAISGLRSEDEADYYCGYWDYSL | |
| | SGYVLGGGTKLTVL | |
| 181 | QSVLTQPPSASGTPGQRVTISCSGSSSNIGSNTFNWYQQLPGTAPKLLI | AJOU-80-VL |
| | YADSHRPSGVPDRFSGSKSGTSASLAISGLRSEDEADYYCGTWDYSL | |
| | SGYVLGGGTKLTVL | |
| 182 | QSVLTQPPSASGTPGQRVTISCSGSSANSRTDGFNWYQQLPGTAPKL | AJOU-86-VL |
| | LIYADSHRPSGVPDRFSGSKSGTSASLAISGLRSEDEADYYCGTWDY | |
| | SLSGYVLGGGTKLTVLG | |
| 183 | QSVLTQPPSASGTPGQRVTISCSGSAQFGSRDNFNWYQQLPGTAPKL | AJOU-87-VL |
| | LIYADSHRPSGVPDRFSGSKSGTSASLAISGLRSEDEADYYCGTWDY | |
| | SLSGYVLGGGTKLTVLG | |
| 184 | QSVLTQPPSASGTPGQRVTISCSGSTKQMHNYQFNWYQQLPGTAPK | AJOU-88-VL |
| | LLIYADSHRPSGVPDRFSGSKSGTSASLAISGLRSEDEADYYCGTWD | |
| | YSLSGYVLGGGTKLTVLG | |
| 185 | QSVLTQPPSASGTPGQRVTISCSGSLLRGENLQFNWYQQLPGTAPKL | AJOU-89-VL |
| | LIYADSHRPSGVPDRFSGSKSGTSASLAISGLRSEDEADYYCGTWDY | |
| | SLSGYVLGGGTKLTVLG | |
| 186 | QSVLTQPPSASGTPGQRVTISCSGSPLFPDSGSFNWYQQLPGTAPKLL | AJOU-90-VL |
| | IYADSHRPSGVPDRFSGSKSGTSASLAISGLRSEDEADYYCGTWDYS | |
| | LSGYVLGGGTKLTVLG | |
| 187 | QSVLTQPPSASGTPGQRVTISCSGSAALDLSPSFNWYQQLPGTAPKLL | AJOU-91-VL |
| | IYADSHRPSGVPDRFSGSKSGTSASLAISGLRSEDEADYYCGTWDYS | |
| | LSGYVLGGGTKLTVLG | |
| 188 | QVQLVQSGAEVKKPGASVKVSCKASGYTFTNYGISWVRQAPGQGL | REGN-VH-3 |
| | EWMGWISVYNGKTNYAQKLQGRVTMTTDTSTTTAYMEMRSLRSD | |
| | DTAVYYCARGSGYDLDYWGQGTLVSVSS | |
| 189 | EVQLVESGGGLVQPGGSLRLSCAASGFTFSSFWMTWVRQAPGKGLE | REGN-VH-19 |
| | WVANIKQDGSEKYYVDSVKGRFTISRDNAKNSLYLQMNSLRAEDT | |
| | AVYYCARDPGRTMVRGGIRYYYGMDVWGQGTTVTVSS | |
| 190 | EVKLAESGGGLVQPGGSLRLSCAASGFTFSSHWMNWVRQAPGKGL | REGN-VH-35 |
| | EWVANIKQDGSDKYYVDSVKGRFTISRDNAKNSLYLQLNSLIAEDT | |
| | AVYYCARDRGVRPPRGAFDIWGQGTMVTVSS | |
| 191 | QVQLVQSGAEVKKPGASVKVSCKASGYTFNSYGISWVRQAPGQGL | REGN-VH-51 |
| | EWMGWIRTYNGNTNYAQKLQGRVTMTTDTSTSTAYMELRSLRSDD | |
| | TAVYYCARDEARIVVAGTTPYYYGMDVWGQGTTVTVSS | |
| 192 | QVQLVESGGGLVQPGGSLRLSCAVSGFTISDHYMSWIRQAPGKGLE | REGN-VH-67 |
| | WISYISSSGSKIYYADSVKGRFTISRDNAKNSLFLQMNSLRAEDTAVY | |
| | YCARTRQLVGDYWGQGTLVTVSS | |
| 193 | EVQLVESGGGLVQPGRSLRLSCAASGFTFDNYAMHWVRQAPGKGL | REGN-VH-83 |
| | EWVSGIRWNSGSIGYADSVKGRFTISRDNAKNSLYLQMNSLRAEDT | |
| | ALYYCAKEGGYSGYRPGPFFDYWGQGTLVTVSS | |
| 194 | QVQLVQSGAEVKKPGASVKVSCKASGYTFTNYGISWVRQAPGQGL | REGN-VH-99 |
| | EWMGWISVYNGHTNYAQKLQGRVTMTTDTSTSTAYMELRSLRSDD | |
| | TAVYYCARGSGYDFDSWGQGTLVTVSS | |
| 195 | QVQLVQSGAEVKKPGASVKVSCKASRYTFTSYDINWVRQATGQGL | REGN-VH-115 |
| | EWMGWMNPNSGNTGYAQKFQGRVTMTRNTSTSTAYMELSSLRSE | |
| | DTAVYYCARVRRFFDYWGQGTLVTVSS | |
| 196 | QVQLVQSGPEVKKPGASVKVSCKASGYTFTNYGISWVRQAPGQGLE | REGN-VH-147 |
| | WMGWISVYNGNINYAQKLQGRVTMTTDTSTSTAYMDLRSLRSDDT | |
| | AVYYCARGSGYDFDYWGQGTLVTVSS | |
| 197 | QVQLVQSGAEVKKPGASVKVSCKDSAYTFNRYGISWVRQAPGQGL | REGN-VH-163 |
| | EWMGWISAYTGNTVYAQKLQGRVTMTTDNSTSTAYMELRSLRSDD | |
| | TAVYYCARDKSIFGVVRGFDYWGQGTLVTVSS | |
| 198 | AIQMTQSPSSLSASVGDRVTITCRASQGIRNALGWYQQKPGKAPKLL | REGN-VL-11 |
| | IYAASSLQSGVPSRFSGSGSGTDFTLTFSSLQPEDFATYYCLQDFNYP | |
| | YTFGQGTKLEIK | |
| 199 | DIQMTQSPSSVSASVGDRVTISCRASQGVSSWLAWYQQKPGNAPKL | REGN-VL-27 |
| | LISAASSIQSGVPSRFSGSGSGTDFTLTISSLQPEDFATYYCQQANSFPL | |
| | TFGGGTKVEIK | |
| 200 | DIQMTQSPSSVSASVGDRVTITCRASQGISSWLAWYQQKPGKAPKLL | REGN-VL-43 |
| | IYAASSFQSGVPSRFSGSGSGTDFTLTISSLQPEDFATYFCQQANSFPL | |
| | TFGGGTTVEIK | |
| 201 | DIQMTQSPSSVSASVGDRVTITCRASQDISIWLAWYQQSPGKAPKLLI | REGN-VL-59 |
| | NVASRLQSGVPSRFSGSGSGTDFTLTINSLQPEDFVTYYCQQANSFPI | |
| | TFGQGTRLATK | |
| 202 | DIQLTQSPSFLSASVGDRVTITCWASQGISSYLAWYQQKPGKAPKLLI | REGN-VL-75 |
| | FAASTLQSGVPSRFSGSGSGTEFTLTISSLQPEDFATYYCQQLNSYPLT | |
| | FGGGTKVEIR | |
| 203 | EIVMTQSPATLSVSPGERATLSCRASQSVNYNLAWYQHKPGQAPRL | REGN-VL-91 |
| | LIYGASTRATGIPARFSGSGSGTEFTLTISSLQSEDFAVYYCQQYNNW | |
| | PLTFGGGTKVEIK | |
| 204 | AIQMTQSSSSLSASVGDRVTITCRASQAIRNALGWYQQKPGKAPKVL | REGN-VL-107 |
| | IYAASSLQSGIPSRFSGSGSGTDFTLTISSLQPEDFATYYCLQDYDYPY | |
| | TFGQGTKLEIK | |
| 205 | DIQLTQSPSFLSASVGDRVTITCWASQGIISYLAWYQQKPGKAPKLLI | REGN-VL-123 |
| | YAASTLHSGVPSRFSGSGSGTEFTLTISSLQPEDFATYYCHQLKSYPIT | |
| | FGQGTRLEIK | |
| 206 | AIQMTQSPSSLSASVGDRVTITCRASQDIRNALGWYQQKPGKAPKLL | REGN-VL-155 |
| | IYAASSLQSGVPSRFSGSASGTDFTLTISSLQPEDFAAYYCLQDYNYP | |
| | YTFGQGTKLEIK | |
| 207 | EIVMTQSPVTLSLSPGERATLPCRASQSVSSSLAWYQQKAGQSPRLLI | REGN-VL-171 |
| | YGASTRATGIPARFSGSGSGTEFTLTISNLQSEDFAVYYCQQYNNWP | |
| | LTFGGGTKVEIK | |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Determination of Subvisible Particles

For the determination of subvisible particles, suitable methods include “Method 1” (Light Obscuration Particle Count Test) and “Method 2” (Microscopic Particle Count Test). Using light obscuration, the FDA requirement for subvisible particulates in parenteral drug product is ≤6,000 particles per container for particles≥10 micrometers in diameter, and ≤600 particles per container for particles>25 micrometers in diameter. Using the microscopic method, the FDA requirement for subvisible particulates in parenteral drug product is ≤3,000 particles per container for particles≥10 micrometers in diameter, and ≤300 particles per container for particles≥25 micrometers in diameter. Presently, no specification exists for particles of less than 10 micrometers in diameter, but the FDA has requested that particles of 2 to 10 micrometers be measured.

Particles of greater than 1 micrometer in diameter were measured using HIAC light obscuration and Brightwell micro-flow imaging (MFI). HIAC combines light obscuration with laser light scattering enabling the detection and counting of particles ranging from 500 nm-350 μm in a moving fluid stream. Particles were sized based on voltage response generated in the detector and sorted into pre-determined size ranges based on voltage response.

For HIAC assays, samples from a manufacturing line (GMP lots) containing a monoclonal antibody at 150 mg/mL were pooled to a total volume of 25 mL. For each pooled sample, three readings of five milliliters per sample were made. Laboratory samples of the same 150 mg/mL antibody formulation were also examined by HIAC. Samples from at least three vials (2.5 mL/vial), seven 1-mL syringes (1.14 mL/syringe), or five 2.25-mL syringes (2 mL/syringe) were pooled, and three reading of one milliliter per reading were made. HIAC 9703 and HIAC 8000A instruments (Hach Company, Loveland, Colo.) using the HRLD 400 probe (which reads up to 18,000 cumulative counts per mL) and MC05 probe (which reads up to 10,000 cumulative counts per mL) respectively, were used to make the light obscuration readings.

The MFI method used less material (i.e., 1 mL of formulation, or 1 stability vial or syringe) than HIAC light obscuration and yielded higher particulate numbers than HIAC. Since MFI is microscopy-based, that method was more sensitive to the translucent protein particulates and was able to differentiate silicone oil droplets/air bubbles from protein particulates for prefilled syringe samples. MFI was conducted on a laboratory sample containing 150 mg/mL of a monoclonal antibody (as in the HIAC analyses). For MFI, one reading of one milliliter per reading was made.

### Determination of Polysorbate Degradation

Degradation of polysorbate was examined using one or more of several methods. The first method employed an enzymatic colorimetric assay to quantify non-esterified fatty acids (NEFA). The NEFA-HR(2) kit (Wako Diagnostics, Richmond, Va.) was used to detect fatty acids in formulated drug substance containing polysorbate. Briefly, the samples were combined with ATP and coenzyme A (CoA) in the presence of acyl-CoA synthetase (ACS). Available (free) fatty acids reacted with the CoA to form acyl-CoA. The acyl-CoA product was reacted with oxygen and acyl-CoA oxidase to produce trans-2,3-dehydroacyl-CoA and hydrogen peroxide. Peroxidase catalyzed the reaction of the hydrogen peroxide with 4-aminoantipyrine and 3-methyl-N-ethyl-N-(B-hydroxyethyl)-aniline to form a blue purple pigment (maximum absorbance at 550 nm). The amount of NEFA in the sample is proportional to the amount of pigment. For a detailed description of the NEFA colorimetric assay, see Duncombe, “The Colorimetric Micro-Determination of Non-Esterified Fatty Acids in Plasma,” Clin Chim Acta. 9:122-5 (1964); Itaya and Ui, “Colorimetric Determination of Free Fatty Acids in Biological Fluids,” J. Lipid Res. 6:16-20 (1965); Novak, M., “Colorimetric Ultramicro Method for the Determination of Free Fatty Acids,” J. Lipid Res. 6:431-3 (1965); and Elphick, M. C., “Modified Colorimetric Ultramicro Method for Estimating NEFA in Serum,” J. Clin. Pathol. 21(5):567-70 (1968).

The test sample containing the protein of interest (and putative host cell protein contaminant) was applied to a 10 kDa molecular weight cut-off filter. The retentate was recovered in 10 mM histidine (pH 6.0) at greater than 100 g/L protein and spiked with polysorbate to give a test sample of 100 g/L protein, 0.8% (w/v) polysorbate, 10 mM histidine, pH 6.0 (tᵢₙᵢₜᵢₐₗ). The test sample was subjected to 45° C. for 44 hours (t_{final}). Some samples were spiked with oleic acid to evaluate the recovery efficiency of NEFA in the samples. Percent polysorbate degradation was calculated as follows:

$$\frac{( [ N E F A ] {t}_{final} - [ N E F A ] {t}_{initial} )}{[ p o l y s o r b a t e ] - [ N E F A ] {t}_{final}} \times 100 \%$$

The second method for determining polysorbate degradation was based on mass spectroscopy. Using LC/MS analysis, this assay allowed the measurement and comparison of the initial percentage of esters and remaining percentage of esters in polysorbates after incubation at 45° C. at different time points. MAb1 produced according to process 6 (without HIC and with PS degradation activity) and mAb1 produced according to process 3 (with HIC step and without PS degradation activity) (see Example 4 and Table 9) were included as negative and positive controls, respectively.

Briefly, 15 mg of antibody sample (on the order of 5-10 mg/mL, or 7 mg/mL±1.5 mg/mL) was applied to an ultra-filter (Amicon Ultra 50K, Millipore, Billerica, Mass.) and centrifuged at 14,000×g for 15 minutes or until the remaining volume was slightly below the 100 μL marking on the device. 1 μL of 10% polysorbate was added into the spin filter with the concentrated protein followed with vortexing. The sample was recovered by inverted centrifugation for 5 minutes at 1000 g to recover the full volume in the collection tube.

The recovered volume was measured and the concentration of polysorbate calculated. 1 μL of each recovered sample was and diluted 100-fold in a separate tube, and the protein concentration measured with Nanodrop 1000 (Thermo Fisher Scientific, Inc., Wilmington, Del.). The samples were then diluted in histidine buffer (10 mM, pH 6.0) and polysorbate stock to achieve 150 mg/mL protein concentration and 0.2% (w/w) polysorbate concentration.

Time zero (TO) sample (2 μL) was reserved from each sample and stored at −80° C. until used. Samples to be tested were sealed under argon and incubated at 45° C. to induce degradation, and removed for testing at the prescribed time points. 2 μL was taken from each of the samples at each time point and diluted with water to 100 μL. Each diluted time point sample was stored at −80° ° C. storage. After collection of each time point, the head space of the sample tube was filled with argon gas, the sample container resealed, and the sample returned to the incubator to resume incubation.

The time point samples were analyzed using an anion exchange column (Oasis MAX column, 30 μm, 2.1 mm×20 mm; Waters Corporation, Milford, Mass.) followed at t=5 minutes with reverse phase chromatography (ACQUITY UPLC® BEH 130 C4 column, 1.7 μm, 2.1 mm×50 mm; Waters Corporation, Milford, Mass.). The reverse phase output was connected to a mass spectrometer (Thermo Q-Exactive mass spectrometer; Thermo Fisher Scientific, Inc., Wilmington, Del.). The chromatographic conditions are described in Table 2.

The system was equilibrated with 99% mobile phase A (0.1% formic acid in water) at a flow rate of 0.1 mL/minute for 40 minutes prior to first injection. Water was used as a blank injection. The mass spectrometer parameters were as follows: mass range 150-2000 m z; heater temperature at 250° C.; voltage 3.8 kv; sheath gas 40; auxiliary gas 10; capillary temperature 350° C.; and S-lens 50. When mass spectrometry-based identification was not necessary, charged aerosol detection (CAD) was used an analytical flow rate and a desolvation temperature at 100° C.(Lisa et al., “Quantitation of triacylglycerols from plant oils using charged aerosol detection with gradient compensation,” J Chromatogr A. 1176(1-2): 135-42 (2007); Plante et al., “The use of charged aerosol detection with HPLC for the measurement of lipids,” Methods Mol Biol. 579:469-82 (2009)).

TABLE 2
Chromatography conditions for determination
of polysorbate degradation

| UPLC System | Waters ACQUITY UPLC I-Class/Dionex UltiMate 3000 | | | | |
|---|---|---|---|---|---|
| Mobile Phase | A: 0.1% formic acid in water | | | | |
| | B: 0.1% formic acid in acetonitrile | | | | |
| Column | Waters Oasis ® MAC 30 μm, 2.1 × 20 mm, | | | | |
| | Part No. 186002052 ACQUITY UPLC ® BEH130 | | | | |
| | C4 column, 1.7 μm, 2.1 mm × 50 mm from Waters, | | | | |
| | Part No. 186004496 | | | | |
| Column | 40° C. ± 1° C. | | | | |
| Temperature | | | | | |
| Autosampler | 5° C. ± 2° C. | | | | |
| Temperature | | | | | |
| Injection | 20.0 μL | | | | |
| Volume | | | | | |
| | Time | | | Flow | |
| | (minute) | % A | % B | (μL/minute) | Curve |
| Gradient | Initial | 99.0 | 1.0 | 100 | Initial |
| | 1.0 | 99.0 | 1.0 | 100 | Linear |
| | 5.0 | 85.0 | 15.0 | 100 | Linear |
| | 40.0 | 1.0 | 99.0 | 100 | Linear |
| | 45.0 | 1.0 | 99.0 | 100 | Linear |
| | 45.1 | 99.0 | 1.0 | 100 | Linear |
| | 50.0 | 99.0 | 1.0 | 100 | Linear |

To estimate the total amount of polyoxyethylene (POE), the mass chromatogram was extracted using the 300-800 m z range to avoid interference from degraded proteins, and the cluster of peaks from about 8-15 minutes was integrated. For CAD chromatograms, the first cluster of POE peaks was directly integrated from about 8-15 minutes (again, retention time may shift slightly). When there were other species co-eluting with the POE, the baseline was adjusted to minimize their impact on the peak area.

To estimate the total amount of POE esters, the mass chromatogram was extracted using the 300-2000 m z range, and the cluster of peaks from about 17-40 minutes was integrated. For the CAD chromatograms, the POE esters peak cluster was directly integrated from about 17-40 minutes.

Percentage of POE esters was calculated according to the following equation:

$$\frac{POE esters peak area}{POE esters peak area + POE area} \times 100 \%$$

Percentage of remaining POE esters was calculated according to the following equation:

$$\frac{\% POE esters at tn}{\% POE esters at t 0}$$

wherein n=2, 4, or 10 days.

### Example 1. Failure of Particulate Specification

Two GMP lots of the 150 mg/mL antibody formulation were assessed for subvisible particles via HIAC light obscuration after at least six months storage at 5° C., as described in U.S. Pat. No. 10,342,876, which is herein incorporated by reference. The formulation comprised 0.02% polysorbate 20 from supplier A, and 150 mg/mL anti-IL-4R antibody. The antibody was purified from CHO cell culture using a combination of affinity capture and ion exchange chromatography. The results are presented in Table 3.

TABLE 3
Number of particulates ≥10 μm in size after storage

| | | | | | 25° C. |
|---|---|---|---|---|---|
| | 5° C. Storage | | | | Storage |
| Lot | 0 months | 6 months | 9 months | 12 months | 6 months |
| 1 | 125 | 6,299 | 13,361 | 29,505 | 32,744 |
| 2 | 18 | 353 | 8,027 | 10,797 | 18,602 |

### Example 2. Quality and Purity of Fatty Acid Ester Affects SVP Formation

The effect of the nature and quality of the non-ionic detergent (polysorbate 20 and polysorbate 80) on subvisible particle formation in a protein formulation was tested by formulating an antibody in either (i) polysorbate 20 from supplier A (PS20-A), (ii) polysorbate 20 from suppler B (PS20-B), or (iii) polysorbate 80 (PS80), as described in U.S. Pat. No. 10,342,876, which is herein incorporated by reference. Table 4 shows HIAC SVP (≥10 μm SVPs) data from the formulated drug substance of the following formula: 20 mM histidine (pH 5.9), 12.5 mM acetate, 0.02% non-ionic detergent (polysorbate), 5% sucrose (w/v), 25 mM arginine, and 150 mg/mL antibody, stored as 2.5 mL fill in a 5 mL Type 1 borosilicate glass vial with a West S2-F451 4432/50 GRY B2-40 stopper.

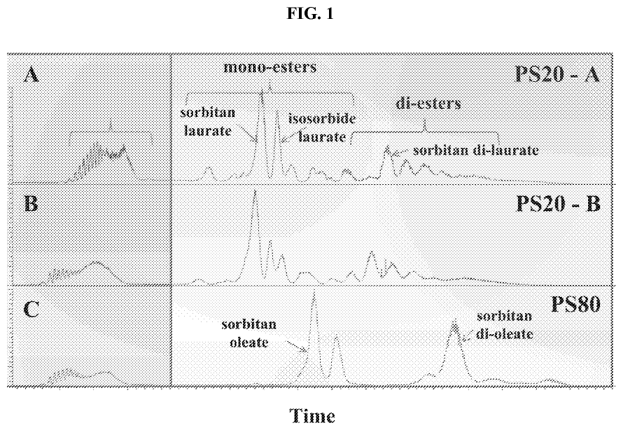

Here, formulated drug substance (“mAb1”) containing polysorbate 80 showed significantly less SVP formation over time than those formulations containing polysorbate 20. Furthermore, formulations containing polysorbate 20 from supplier B (PS20-B), which is a higher grade of polysorbate 20, showed less SVP formation than those formulations containing polysorbate 20 from supplier A (PS20-A; a lower grade of polysorbate 20). A comparative analysis of PS20-A and PS20-B shows that PS20-B has 5-10% more overall esters than PS20-A, and that PS20-A has more isosorbide laurate ester than does PS20-B, as shown in FIG. 1.

TABLE 4
Number of particulates ≥10 μm in size
after storage with varying detergents

| | | 5° C. Storage | | | | 25° C. |
|---|---|---|---|---|---|---|
| | Non-ionic | 0 | 6 | 9 | 12 | Storage |
| Lot | detergent | months | months | months | months | 6 months |
| 1 | PS20-A | 125 | 6,299 | 13,361 | 29,505 | 32,744 |
| 2 | PS20-A | 18 | 353 | 8,027 | 10,797 | 18,602 |
| 1 | PS20-B | 25 | 175 | NA | 1,138 | 4,221 |
| 2 | PS20-B | 8 | 108 | NA | 1,198 | 5,682 |
| 1 | PS80 | 19 | 26 | NA | 22 | 92 |

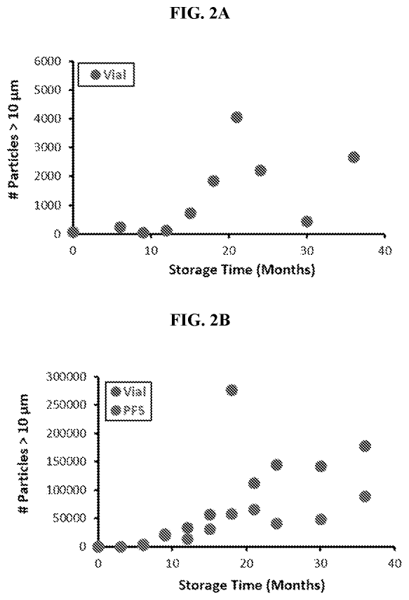

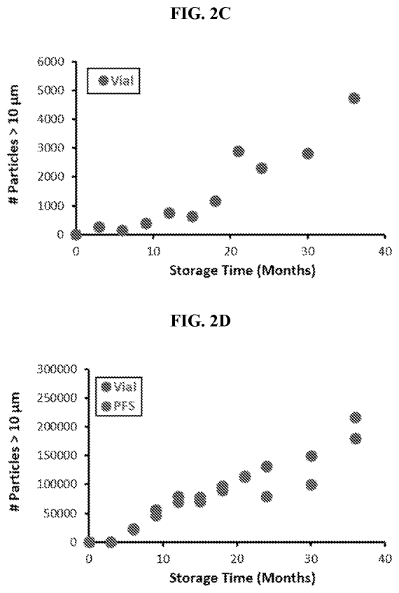

Formulated drug substance comprising HP-PS20 or SR-PS80 was further assessed using a membrane microscopy method or MFI after storage for up to 36 months, as shown in FIG. 2. Number of particles>10 μm was additionally compared between formulations stored in a glass vial compared to formulations stored in a pre-filled syringe, as shown in FIGS. 2A, 2B, 2C and 2D. In all cases, the number of particles increased substantially over time.

It was hypothesized that degradation of the fatty acid ester of polysorbate in the formulations may promote protein instability and consequently result in SVP formation. In order to assess polysorbate degradation, the stability of polysorbate 20 and polysorbate 80 in the 150 mg/mL antibody (mAb1) formulation containing 0.02% non-ionic detergent (polysorbate) prepared without HIC (process 3, see below and Table 9)) were compared. The relative amounts of remaining esters (mono- and di-esters) were determined by mass spectroscopy. Significant degradation of the ester components of polysorbate 20 was observed after the samples were stored at 5° C. for six months or 45° C. for two months. Less extensive degradation was observed for polysorbate 80 under the same conditions (see Table 5). These results correlate with the SVP particle formation observations.

The rates of degradation of polysorbate 20 and polysorbate 80 formulated with 150 mg/mL antibody (mAb1) (as described above for Table 5) were determined under identical conditions using mass spectroscopy to measure relative amounts of free fatty acids and fatty acid esters. Percent ester degradation was determined using the following formula:

$$\frac{\% POE esters at T 0 - \% POE esters at T 1}{\% POE esters at T 0}$$

wherein T0=time zero, T1=time at experimental condition (i.e., 2 months at 45° C.; 6 months at 5° C.), and POE-polyoxyethylene. Table 6 shows percent degradation of polysorbate 20 and polysorbate 80 in 150 mg/mL antibody formulations. The degradation rate of polysorbate 80 was consistently lower for mAb1 (but not for all antibodies tested) than the degradation rate of polysorbate 20 in otherwise identical antibody formulations.

TABLE 5
Percent remaining esters after storage

| | | | 6 months | 2 months |
|---|---|---|---|---|
| Detergent/chemical entity | | 0 months | at 5° C. | at 45° C. |
| PS20 | Monoester | 100% | 60% | 30% |
| | Diester | 100% | 30% | 10% |
| PS80 | Monoester | 100% | 80% | 35% |
| | Diester | 100% | 75% | 25% |

TABLE 6
Percent ester degradation after storage

| | | 6 months at | 2 months at |
|---|---|---|---|
| Polyoxyethylene ester | 0 months | 5° C. | 45° C. |
| PS20 | 0% | 22% | 63% |
| PS80 | 0% | 13% | 47% |

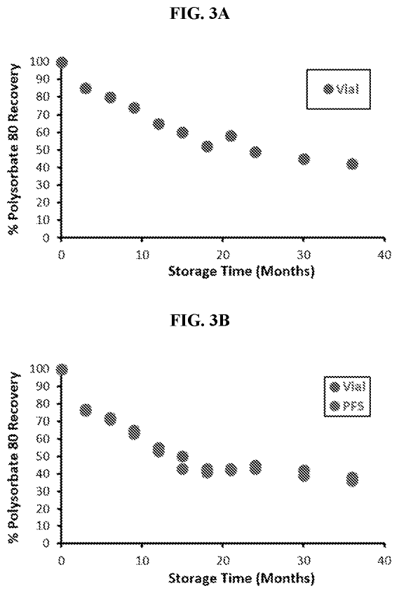

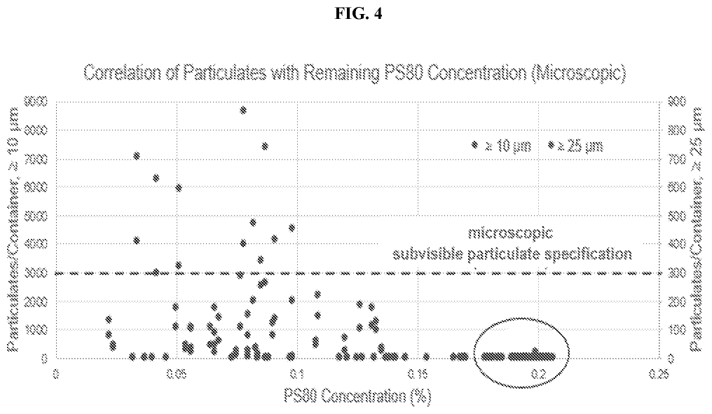

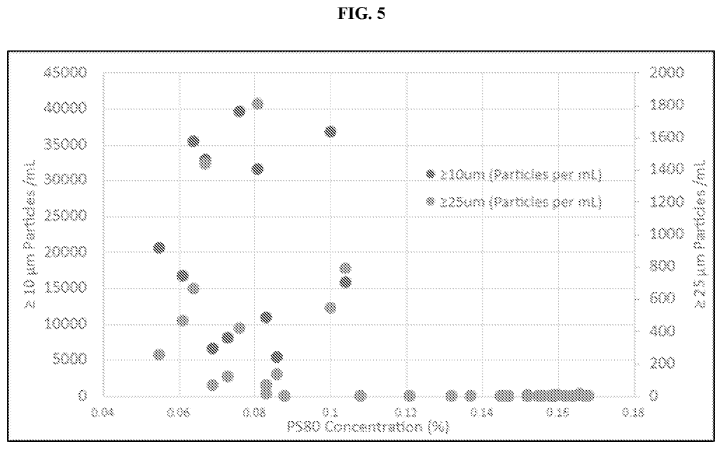

Polysorbate degradation in formulations comprising SR-PS80 or HP-PS20, stored in glass vials or pre-filled syringes, was further compared using CAD-UHPLC, as shown in FIGS. 3A and 3B. In all cases, there was appreciable polysorbate degradation over time. The presence of SVPs correlated with remaining polysorbate concentration in a formulation, as shown in FIG. 4. The same relationship was found using a formulation comprising another monoclonal antibody, mAb5, as shown in FIG. 5. Using Raman spectroscopy, it was confirmed that particulates in polysorbate-containing formulations matched the characteristics of fatty acids. Therefore, in order to solve the problem of SVP formation, the phenomenon of polysorbate degradation and free fatty acid particle formation was further investigated.

### Example 3. Polysorbate Degradation Activity

To determine the etiological agent responsible for polysorbate degradation, the buffered mAb1 antibody (150 mg/mL) was separated into two fractions by 10 kDa filtration: a protein fraction, and a buffer fraction, as described in U.S. Pat. No. 10,342,876, which is herein incorporated by reference. These two fractions, as well as intact buffered antibody, were spiked with 0.2% (w/v) of super refined polysorbate 20 (PS20-B) and stressed at 45° C. for up to 14 days. The study showed that the protein fraction, not the buffer fraction, had an effect on the degradation of sorbitan laurate (i.e., the major component of polysorbate 20), as shown in Table 7, and that the degradation of polysorbate 20 was correlated with the concentration of the antibody, as shown in Table 8.

TABLE 7
Degradation of sorbitan laurate in each mAb1 fraction

| Fraction | Sorbitan laurate % ester remaining (14 days at 45° C.) |
|---|---|
| Drug substance | 75% |
| Protein fraction | 75% |
| Buffer fraction | 100% |

TABLE 8
Degradation of polysorbate in each mAb1 fraction
correlates to antibody concentration

| | Antibody | Polysorbate 20% ester remaining |
|---|---|---|
| Fraction | concentration (mg/mL) | (12 days at 45° C.) |
| Drug | 150 | 82% |
| substance | | |
| Protein | 75 | 92% |
| fraction | | |
| Buffer fraction | 25 | 98% |

### Example 4. Hydrophobic Interaction Chromatography

Antibody was produced in a CHO cell host and purified using one of two processes (see Table 9), as described in U.S. Pat. No. 10,342,876, which is herein incorporated by reference. In one case, the antibody was purified using ion exchangers as polishing steps (capture step, ion exchange 1, ion exchange 2; “Process 3”). In the other case, one of the polishing steps used to purify the antibody was hydrophobic interaction chromatography as an additional polishing step (capture step, ion exchange, hydrophobic interaction; “Process 6”). The antibody, purified by either process 3 or process 6, was formulated at 150 mg/mL in 20 mM histidine (pH 5.9), 12.5 mM acetate, 5% sucrose, 25 mM arginine, and 0.02% polysorbate 20, and subjected to forced degradation at 45° C. for up to 14 days. At day 14, about 98% of the sorbitan laurate (i.e., intact ester) remained in the formulation containing the antibody purified using process 6, whereas only about 28% of the sorbitan laurate remained in the formulation containing the antibody purified using process 3. Therefore, the hydrophobic interaction chromatography (HIC) step likely removed an activity contributing to polysorbate degradation.

TABLE 9
Polysorbate recovery using various antibody purification processes

| Process | | % intact |
|---|---|---|
| no. | Purification steps | PS20 |
| 1 | Protein A affinity capture (PA) | 54% |
| 2 | PA > cation exchange (CEX) | 25% |
| 3 | PA > CEX > anion exchange (AEX) | 86% |
| 4 | PA > CEX > hydrophobic interaction (HIC) | 90% |
| 5 | PA > AEX | 83% |
| 6 | PA > AEX > HIC | 92% |

The role of bulk process steps in removing the putative polysorbate degradation factor (putative esterase activity) was evaluated. Antibody produced from CHO cells was subjected to sequential purification steps, and the stability of polysorbate 20 was assessed at each step. The results from one set of experiments are presented in Table 9, which reports on the percent intact polysorbate 20 at each step or sequence of steps. Percent intact polysorbate 20 is predicted to be inversely proportional to the amount of contaminant esterase activity.

Multiple different antibodies were tested for an associated polysorbate degrading activity (esterase) and the effect of HIC on that activity. In each case, polysorbate 20 degradation activity was detected, and that activity was virtually ablated by the incorporation of a HIC purification step (Table 10).

TABLE 10
Polysorbate degradation with and without HIC purification

| Antibody | HIC or no HIC | PS20 degradation at day 15 |
|---|---|---|
| mAb1 (IgG4) | No HIC | 72% |
| mAb1 (IgG4) | HIC | 2.0% |
| mAb2 (IgG1) | No HIC | 41% |
| mAb2 (IgG1) | HIC | 2.0% |
| mAb3 (IgG4) | No HIC | 40% |
| mAb3 (IgG4) | HIC | 5.0% |
| mAb4 (IgG4) | No HIC | ND |
| mAb4 (IgG4) | HIC | 1.0% |

The role of HIC in subvisible particle formation was explored. Without meaning to be limited by theory, it was hypothesized that the stability of the non-ionic detergent in a protein (e.g., antibody) formulation is directly correlated to the formation of subvisible particles. Loss of surfactant activity may allow protein to aggregate and form subvisible particles. Additionally or alternatively, the fatty acids released by the degrading sorbitan fatty acid esters may also contribute to subvisible particle formation as immiscible fatty acid droplets. Therefore, levels of subvisible particles≥ 10 micrometers in diameter were counted in drug substance (150 mg/mL antibody in 20 mM histidine (pH 5.9), 12.5 mM acetate, 5% sucrose, 25 mM arginine, and 0.02% polysorbate 20) produced with HIC (e.g., process 6) or without HIC (e.g., process 3). The results (presented in Table 11 and Table 12) show that the application of a HIC step significantly reduced the formation of SVPs in the drug substance (on the order of ten-fold less), even though the lower quality PS20-A was used in these experiments.

TABLE 11
Number of particles ≥10 μm in size
with and without HIC purification

| | | | | | 25° C. |
|---|---|---|---|---|---|
| | 5° C. Storage | | | | Storage |
| Process | 0 months | 6 months | 12 months | 24 months | 6 months |
| HIC used | 129 | 189 | 140 | 178 | 136 |
| No HIC | 35 | 769 | 1,342 | 14,346 | 1,951 |

TABLE 12
Number of particles ≥25 μm in size
with and without HIC purification

| | | | | | 25° C. |
|---|---|---|---|---|---|
| | 5° C. Storage | | | | Storage |
| Process | 0 months | 6 months | 12 months | 24 months | 6 months |
| HIC used | 15 | 12 | 28 | 28 | 6 |
| No HIC | 3 | 59 | 30 | 429 | 70 |

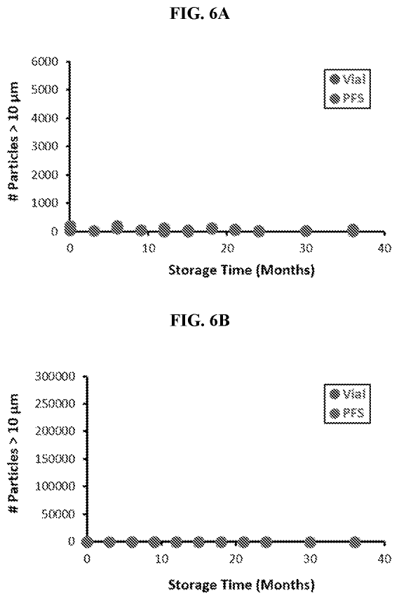

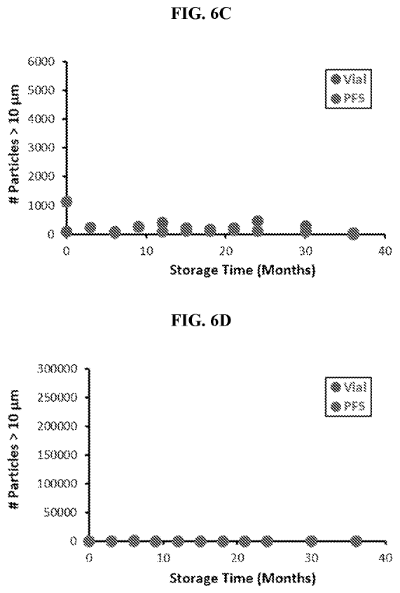

mAb1 formulations comprising PS20 or PS80 and produced with HIC were further characterized over time for the formation of particles, as shown in FIGS. 6A, 6B, 6C and 6D. Formulations comprising either PS20 or PS80, as measured by either membrane microscopy or MFI, did not show an increase in particle formation over time.

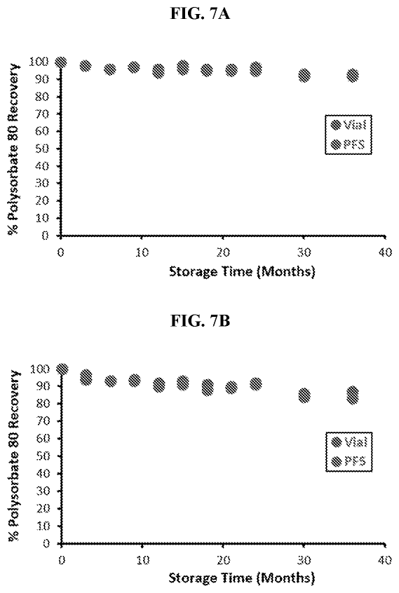

mAb1 formulations produced with HIC were further evaluated for polysorbate degradation over time, as shown in FIGS. 7A and 7B. In agreement with the SVP results described above, formulations subjected to the HIC process did not show appreciable polysorbate degradation even over 36 months of storage.

### Example 5. Putative Phospholipase B-Like 2 Activity

Polysorbate degradation activity was followed during HIC purification of an exemplar antibody produced in CHO cell culture, as described in U.S. Pat. No. 10,342,876, which is herein incorporated by reference. Partially purified CHO cell extract was applied to HIC (phenyl-sepharose). The flow-through, which contained almost all of the antibody, was collected and analyzed for polysorbate degradation activity. No polysorbate degradation activity was observed in this flow-through fraction. The HIC bound fraction was stripped from the HIC media and subsequently subjected to 100 kDa cut-off ultrafiltration/diafiltration. The unfiltered stripped fraction contained 9.9% polysorbate degradation activity, the filter permeate contained 1.3% polysorbate degradation activity and 5% antibody yield, and the filter retentate contained 7.4% polysorbate degradation activity and 95% antibody yield.

TABLE 13
Percent reduction in polysorbate 20 degradation
by concentration of lipase inhibitor

| Lipase inhibitor | 0 mM | 0.001 mM | 0.01 mM | 0.1 mM |
|---|---|---|---|---|
| Orlistat | 0% | 0% | 27.8% | 67% |
| Diethylumbelliferyl | 0% | 48% | 100% | >95% |
| phosphate | | | | |
| URB602 | 0% | 0% | 20% | 0% |
| 2-Butoxyphenyl boronic | 0% | 0% | 28% | 0% |
| acid | | | | |

Whether the polysorbate degrading activity is a lipase was tested by combining a lipase inhibitor with the polysorbate degrading activity fraction spiked with polysorbate 20. Table 13 presents the data showing a reduction of polysorbate degrading activity due to lipase inhibitor relative to the control (antibody with associated polysorbate degrading activity plus polysorbate 20 without lipase inhibitor). Lipase inhibitors reduced or eliminated the polysorbate degradation activity associated with the antibody.

A CHO-produced recombinant antibody HIC strip fraction (not the flow-through), which contained the polysorbate degradation activity, was subjected to additional HIC in bind/elution mode, wherein the antibody was eluted with a shallow gradient. Elution fractions were tested for PS20 degradation activity and those fractions having that activity were subjected to (i) intact mass spectrometry analysis, (ii) native size exclusion chromatography UV analysis (SEC-UV), and (iii) tryptic digestion followed with LC-MS and proteomic search analysis. Intact mass spectrometry analysis of reverse phase liquid chromatography fractions revealed an unknown species in hydrophobic fraction L8 (the most hydrophobic fraction). Formulated antibody samples containing polysorbate 20 and spiked with L8 (1:100) showed 20% polysorbate degradation by day eight. Antibody monomer and free light chain were detected in less hydrophobic fractions L3-L7, as well as L8. Antibody dimer was detected in fractions L5-L8.

HIC strip fractions L3-L9 were subjected to SEC-UV under native conditions. Fraction L8 separated into three major peaks coming off first, and two minor peaks coming off later and representing smaller species. The first peak off the column contained antibody dimer and other oligomers. The second peak contained antibody monomer. The third peak contained the species having polysorbate degradation activity. Thus, the degradation activity is separable from the antibody and is of smaller molecular rotation than the antibody monomer.

HIC fraction L8 was also subjected to shotgun proteomics analysis. Briefly, the L8 fraction was sequentially (i) retained on a 10 kDa filter, (ii) reconstituted in 6M guanidine-HCl, 100 mM Tris-HCl, pH 7.5, (iii) treated for 30 minutes at 50° C. in 10 mM Tris(2-carboxythyl)phosphine hydrochloride) (TCEP) followed by 30 minutes in the dark at room temperature in 20 mM indole-3-acetic acid (IAA), (iv) diluted eight-fold, had trypsin added at 1 part trypsin to 20 parts sample, and incubated at 37° C. for four hours, and then (v) subjected to LC-MS/MS analysis. Proteomic searching of the resultant peptide sequences revealed five proteins associated with L8: (i) putative phospholipase B-like 2 (representing 15% of the peak fraction), (ii) peroxiredoxin-1, (iii) heat shock 27 kDa protein 1, (iv) anaphase-promoting complex subunit 1, and (v) U3 small ribonucleoprotein protein MPP10.

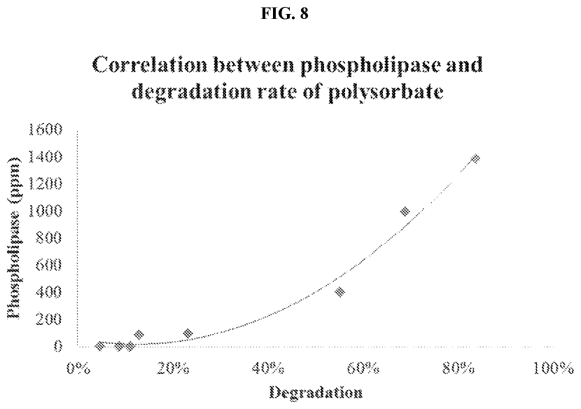

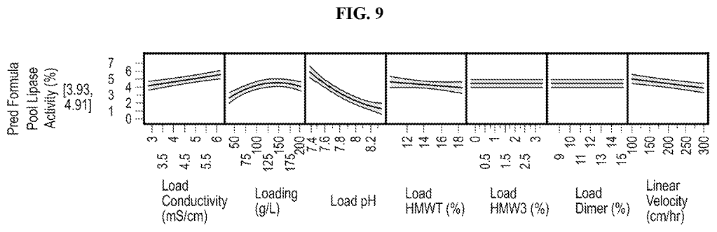

The amount of polysorbate degradation activity correlated with the abundance of phospholipase B-like 2 protein (PLBL2) present. At various purification steps, the amount of PLBL2 was determined via nanoLC-MS or LC-MS and the rate of polysorbate degradation (PS20 spiked fractions) was determined. The abundance of PLBL2 was calculated based on the ratio of peptide intensity from the lipase and drug substance (i.e., antibody). The results are presented in FIG. 8 and Table 14.

TABLE 14
Correlation of PLBL2 and polysorbate degradation

| | | Relative amount |
|---|---|---|
| Fraction | % PS20 degradation | PLBL2 (ppm)² |
| ProA pool | 69.02% | 991 |
| CEX pool | 55.24% | 403 |
| AEX pool | 12.85% | 84 |
| HIC pool | 8.67% | 0 |
| HIC pool 2 | 10.96% | 0 |
| HIC strip | 83.75% | 1384 |
| mAb1 process 3 (example 4) | 4.60% | 0 |
| mAb1 process 6 (example 4)¹ | 29.12% | 92 |

¹Degradation rate adjusted by concentration.
²Abundance of phopholipase calculated based on the ratio of peptide intensity from the lipase and drug substance.

### Example 6. Reduction of Lipase Activity Using AEX

The ability of an anion exchange (AEX) chromatography unit operation to reduce levels of lipases in a drug formulation was investigated. AEX was performed in flow through mode, where negatively charged impurities are adsorbed to the immobilized, positively charged ligand (column), and the product flows through. Transfer functions were generated using stepwise regression for process parameters considered most likely to influence lipase activity. Visualizations of the resulting models are shown in FIG. 9. This model can be used for rational set point and range selection using an optimization algorithm to maximize desirability and robustness of the process. It was surprisingly discovered that lipase activity inversely correlated to AEX load pH, providing a new method for minimized lipase activity by optimizing AEX chromatography conditions.

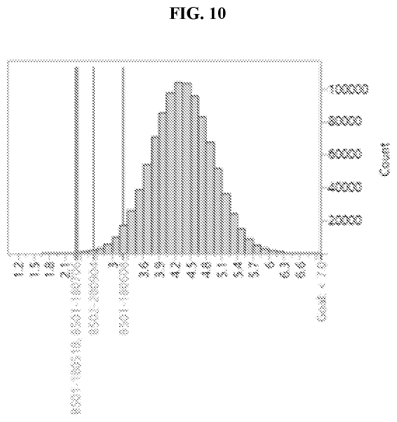

Performance of an optimized AEX process was verified in four pilot-scale (500 L) Confirmation Batches, as shown in Table 15. Lipase activity was measured by stressing concentrated samples at 45° C. for 44 hours after a 0.8% w/v polysorbate 20 spike and measuring change in non-esterified fatty acids (NEFA). Anion exchange process performance during these batches was compared to small-scale model predictions derived from Monte Carlo simulations of the process run at set point with estimated variation in input parameters, as shown in FIG. 10. Confirmation batch responses are indicated with a solid orange line. All responses were within the predicted range generated from the scale-down multivariate model for an anti-IL-4R antibody produced using the process of the present invention, illustrating process robustness to scale-up and appropriateness of the small-scale model to predict pilot scale performance.

TABLE 15
Summary of anion exchange step 500 L scale performance
during process confirmation batches

| | | | | | Confirmation | Scale- |
|---|---|---|---|---|---|---|
| | | | | | Batch | down |
| | | | | | (Mean ± | (Mean ± |
| Bioreactor | B1 | B2 | B3 | B4 | SD, N = 4) | SD) |
| AEX Load Lipase | 6.31 | 6.30 | 6.29 | 7.28 | 6.55 ± 0.49 | N/A |
| Activity (%) | | | | | | |
| AEX Pool Lipase | 2.29 | 3.21 | 2.33 | 2.64 | 2.62 ± 0.42 | 4.3 ± 0.6 |
| Activity (%) | | | | | | |

SD, standard deviation

### Example 7. Optimized Fatty Acid Composition to Reduce Particle Formation

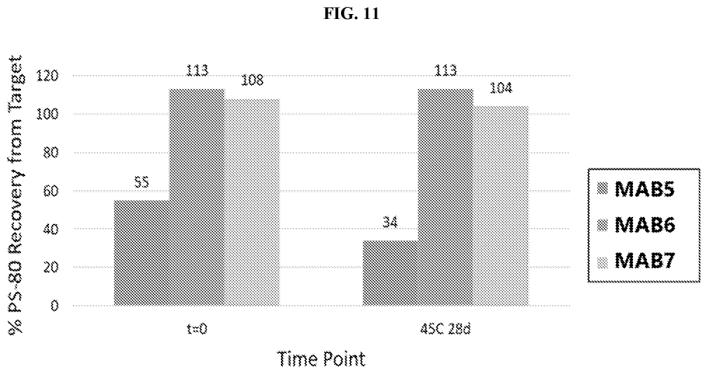

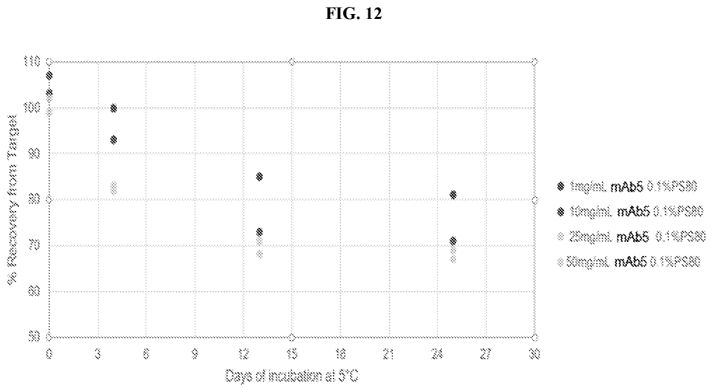

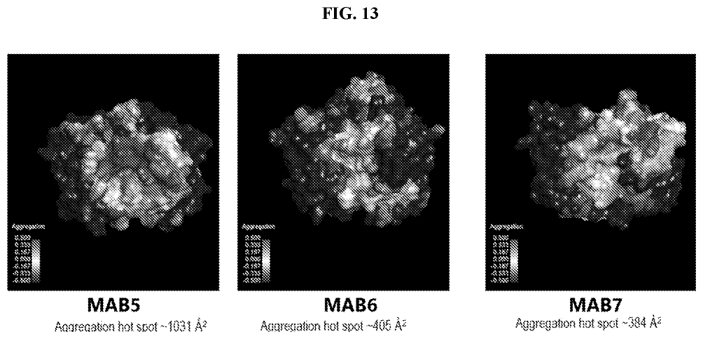

Proteins of interest may present additional challenges to lipase removal based on their structure and physicochemical characteristics. mAb5 was formulated following typical HCP removal techniques, but still showed polysorbate degradation over time in storage, as shown in FIG. 11 and FIG. 12. Homology modeling demonstrated that mAb5 features a large hydrophobic patch compared to mAb6 and mAb7, as shown in FIG. 13, which could cause co-elution of mAb5 with hydrophobic lipases. Therefore, further approaches to reducing polysorbate degradation and SVP formation were investigated.

Fatty acid composition and percent distribution of high melting point components are useful considerations for predicting particulate formation. Free fatty acids with high melting points can likely form insoluble particles that can be detected at room temperature and during analytical assessment. The composition of fatty acids in different polysorbates, along with their respective melting points, is shown in Table 16. Commercially available polysorbates may have fatty acid compositions with a wide range of melting points, with PS80 featuring a higher concentration of low melting point fatty acids, in particular oleic acid.

TABLE 16
Fatty acid compositions of commercially available polysorbates

| | | | | | | Croda High | |
|---|---|---|---|---|---|---|---|
| Fatty | % (from | SR- | Seppic | Croda | NOF PS80 | Oleic SR- | Melting |
| acids | CoA) | PS20 | PS80 | SR-PS80 | (ChP) | PS80 (ChP) | Point (° C.) |
| Capric | ≤10 | 1.7 | — | — | — | — | 31.6 |
| Lauric | 40.0-60.0 | 55.6 | — | — | — | — | 43.2 |
| Myristic | ≤5.0 | 22.8 | 0 | 0 | NMT0.1 | 0 | 54.4 |
| Palmitic | ≤16.0 | 10.8 | 12 | 1.4 | 0.2 | 0.1 | 62.9 |
| Stearic | ≤6.0 | 0.3 | 2.8 | 2.3 | NMT0.1 | 0 | 69.3 |
| Oleic | ≥58.0 (≥98.0 | 6.9 | 70.6 | 87.8 | 99.3 | 99.5 | 13.4/16.3 |
| | ChP) | | | | | | |

The storage stability of mAb1 drug product (DP) prepared without a HIC step was evaluated across samples comprising different grades of PS80, as described in U.S. Patent Application Publication No. 20190083618 A1, which is herein incorporated by reference. Each DP sample had a volume of 2.136 mL, contained the same concentration of mAb1 (150 mg/mL), and 0.2% (w/v) of one of several lots of PS80. Each PS80 lot had one of three different percentage contents of oleic acid ester (70%, 87%, and ≥ 99%). Table 17 summarizes the percentage content of oleic acid ester in the PS80 in each FDS sample.

TABLE 17

| Sample | % oleic acid ester content in PS80 (Lot) | |
|---|---|---|
| DP A | 87% | |
| DP B | ≥99% | (Lot 1) |
| DP C | ≥99% | (Lot 2) |
| DP D | 70% | (Lot 1) |
| DP E | 70% | (Lot 2) |
| DP F | 70% | (Lot 3) |

The DP samples were stored at 2-8° C. in glass pre-filled syringes for up to 24 months. Particulates were measured in each DP sample every six months for a total of 24 months, by both membrane microscopy method and micro-flow imaging (MFI).

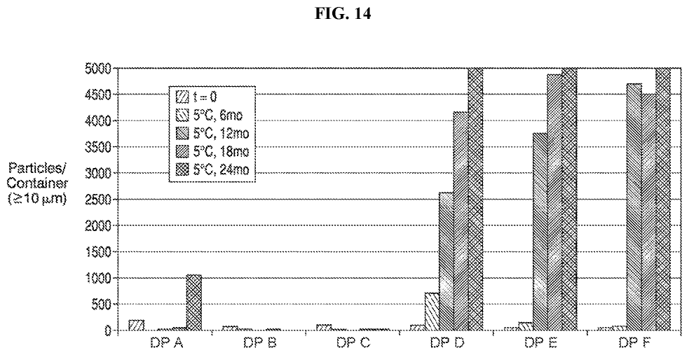

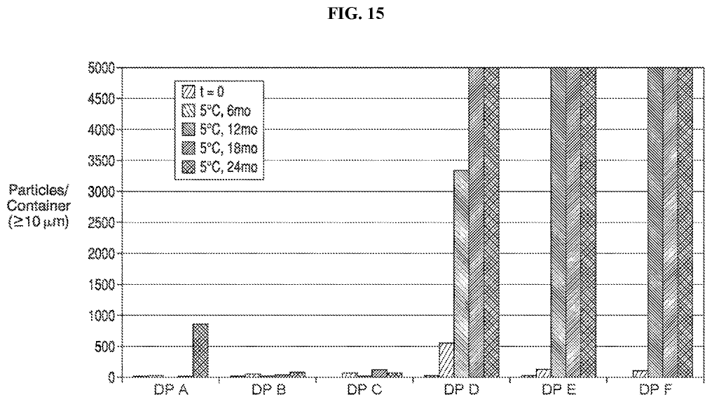

FIG. 14 shows the number of SVPs per container having a diameter of ≥ 10 μm, as measured by membrane microscopy. FIG. 15 shows, in chart form, the number of SVPs per container having a diameter of >10 μm, as measured by MFI. As shown in FIG. 14 and FIG. 15, DP B and DP C (the two DP samples containing PS80 having a≥ 99% content of oleic acid esters) displayed the lowest numbers of SVPs across the full 24-month period, as measured by both membrane microscopy (FIG. 14) and MFI (FIG. 15). DP A, containing PS80 having an 87% content of oleic acid esters, displayed the next lowest number of subvisible particulates across the 24-month period (in particular, showing between 800 and 1200 particles by 24 months). DPs D, E, and F all showed well over 3000 particles per container (as measured by both methods) by at least the 18-month mark.

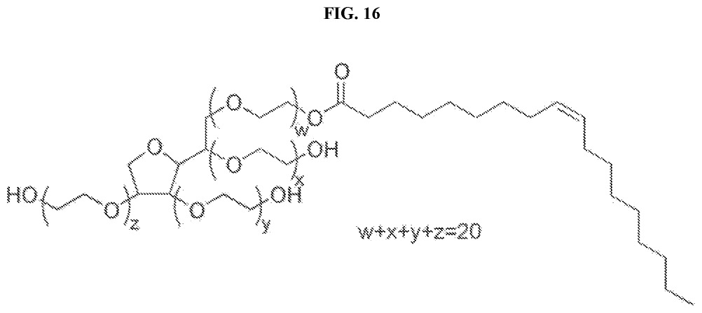

It was hypothesized that the lower numbers of particles in DPs A, B, and C (as compared to the more numerous particles in DPs D, E, and F) were a result of the use of PS80 having a higher percentage content of oleic acid (or long-chain fatty acid) esters. Oleic acid is a longer chain fatty acid, with one unsaturated bond (see FIG. 16). Therefore, it has a sub-ambient melting temperature of about 13° C. A precursor to subvisible and visible free fatty acid (FFA) particulate formation is the agglomeration of individual FFA chains into aggregates, which then precipitate in the form of particles. Oleic acid may be generated during storage of the formulations at 5° C. by, e.g., enzymatic hydrolysis of the fatty acid esters in polysorbate 80. This oleic acid may form SVPs, but due to its low melting temperature, such particles are more likely to exist as an oily liquid in protein formulations at room temperature (about 22° C.) where analysis is performed, and therefore does not persist as subvisible particulates at room temperature. As a contrast, higher amounts of non-oleic acid ester content in the formulation will lead to formation of their corresponding FFA upon hydrolysis, and due to their higher melting temperatures the subvisible and visible amorphous particulates thus formed persist at ambient temperature during analysis.

Additionally, oleic acid esters are better solubilizing/stabilizing agents than esters of shorter chain fatty acids due to oleic acid esters' higher hydrophobicity, which enables oleic acid esters to solubilize free fatty acid and protein particulates, thereby maintaining product stability. Therefore, polysorbate 80 with higher contents of oleic acid esters (>98%) can provide improved stability to protein formulations and drug products as compared to polysorbate 80 with lower contents of oleic acid esters.

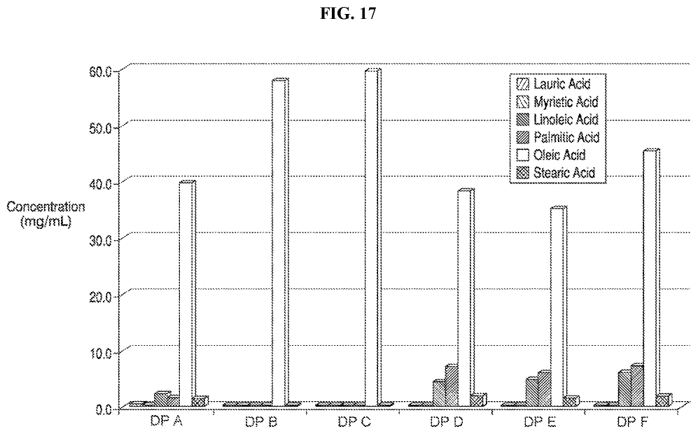

A concentration of each type of free fatty acid (in micrograms/mL) in each sample DP (DP A-F) was evaluated after storage of the samples at 5° ° C. for 18 months. Sample DP A-F were prepared as described above. Free fatty acid concentrations were measured at 18 months by LC-MS, as shown in FIG. 17. DPs B and C (the two DP samples containing PS80 having a≥ 99% content of oleic acid esters displayed the highest concentration of oleic acid, and the lowest concentrations of other FFAs. This indicates the homogeneity of the FFAs (i.e., oleic acids) in DPs B and C. This further indicates that the use of polysorbate comprising a high oleic acid concentration can reduce the formation of free fatty acid particles at ambient temperature even in a formulation comprising a lipase that causes free fatty acid production.

### Example 8. Prevention of Particle Formation in Lipase-Containing Formulations with PEG3350 or Poloxamer 188

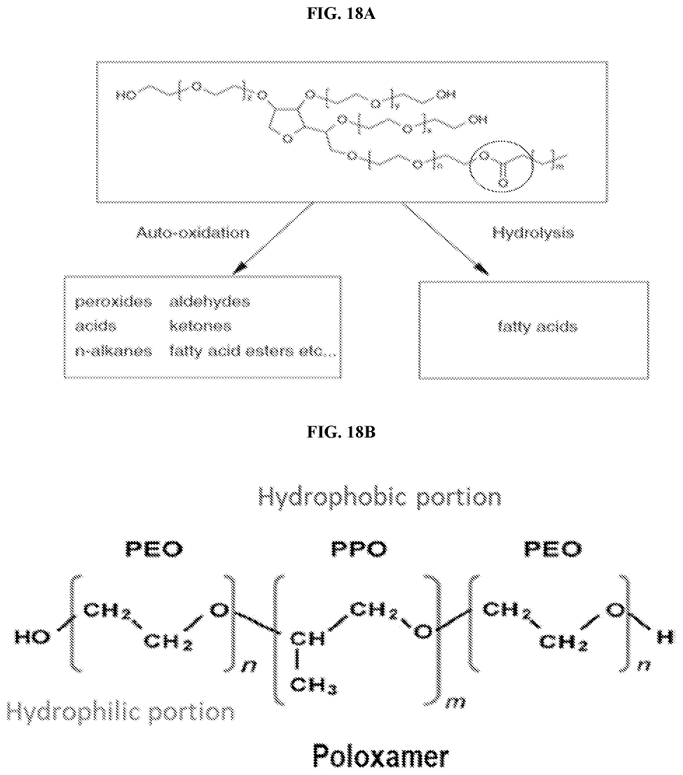

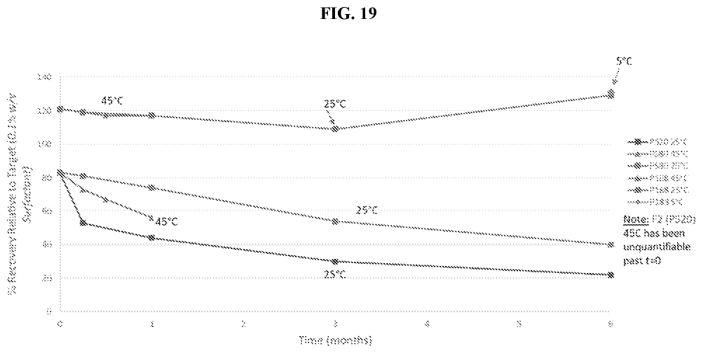

Esterases or lipases can hydrolyze polysorbates by enzymatic hydrolysis of the ester bond, as shown in FIG. 18A. Alternative surfactants such as PEG3350 and poloxamer 188 don't contain ester bonds and therefore are not targets for esterases, as shown in FIG. 18B. mAb5 was formulated with PS20, PS80, or poloxamer 188, and the recovery of surfactant was compared, as shown in FIG. 19. Unlike the PS20 and PS80 formulations, poloxamer formulations did not experience recovery losses, at all temperatures tested.

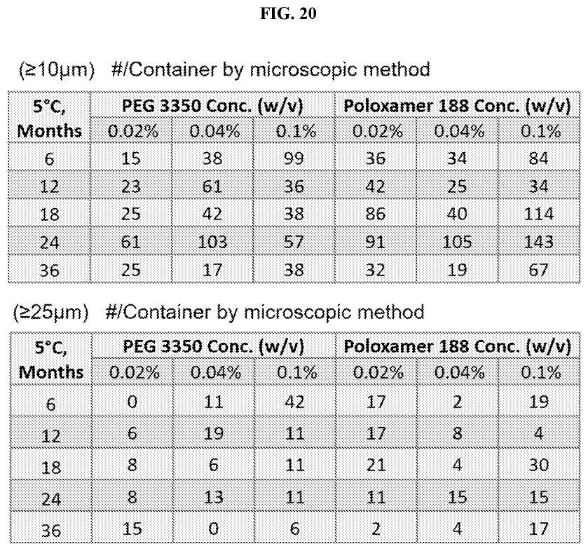

In order to investigate whether the use of alternative surfactants could reduce the formation of SVPs in a formulated DP, formulations of IL-4R antibody were prepared using alternative surfactants, and particulate formation was measured over time, as described in U.S. Provisional Application No. 63/337,532, which is herein incorporated by reference. An anti-IL-4R antibody comprising the HCVR/LCVR amino acid sequence pair of SEQ ID NOs: ½ at a concentration of 150 mg/mL was formulated with 20 mM histidine, 12.5 mM sodium acetate, 25 mM arginine-HCl, 5% w/v sucrose, and either PEG3350 or poloxamer 188 at varying concentrations, at pH 5.9. The formulations were stored in syringes at 5° ° C. for up to 36 months with periodic measurements taken of the number of particles (≥ 10 μm and ≥ 25 μm) present in the formulations, as determined by microscopy. As shown in FIG. 20, few particles of ≥ 10 μm or ≥ 25 μm were identified in the formulations over the 36 month observation period. Moreover, no appreciable differences or changes in the number of subvisible particles among the different PEG3350—or poloxamer 188-containing formulations were observed over the course of the storage period.

These results demonstrate that alternative surfactants such as poloxamer 188 are resistant to degradation in a lipase-containing formulation, and that low concentrations of PEG3350 or poloxamer 188 can prevent particle formation in a DP formulation over long storage periods.

In order to assess the capacity of alternative surfactants to promote therapeutic protein stability, formulations comprising PEG3350 or poloxamer 188 were further subjected to assessments of agitation stress stability and thermal stress stability. The agitation stress stability was tested for IL-4R antibody formulations containing different concentrations of the surfactant PEG3350 or poloxamer 188. An anti-IL-4R antibody comprising the HCVR/LCVR amino acid sequence pair of SEQ ID NOs: ½ at a concentration of 150 mg/mL was formulated with 20 mM histidine, 12.5 mM sodium acetate, 25 mM arginine-HCl, 5% w/v sucrose, and PEG3350 or poloxamer 188 at varying concentrations, at pH 5.9. The formulations were stored in glass vials and agitated by vortexing (speed setting=4) for 30 minutes, 60 minutes, or 120 minutes. The percentage of high molecular weight (HMW) species was then determined by size exclusion ultra high performance liquid chromatography (SE-UPLC).

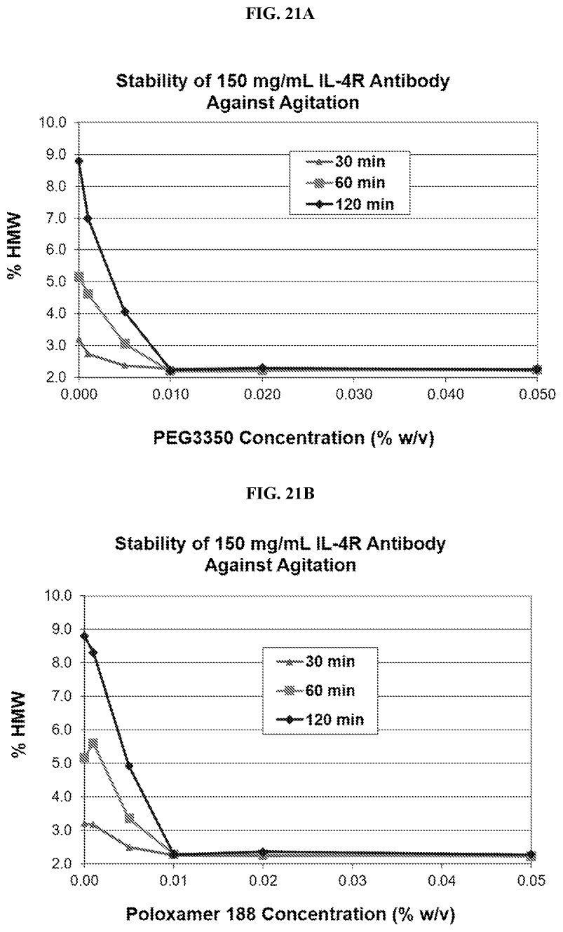

As shown in FIG. 21A, formulation of the antibody with at least 0.01% (w/v) PEG3350 prevented an observable increase of HMW species (quantitated by SE-UHPLC) due to the agitation. Lower amounts of PEG3350 (0.001% or 0.005%) were insufficient to prevent formation of HMW. Similarly, as shown in FIG. 21B, formulation of the antibody with at least 0.01% (w/v) poloxamer 188 prevented an observable increase of HMW species, but HMW species were observed with lower amounts of poloxamer 188 (0.001% or 0.005%).

The thermal stress stability of IL-4R antibody formulations containing PEG3350 or poloxamer 188 was additionally tested and compared to various IL-4R antibody formulations containing polysorbate. An anti-IL-4R antibody comprising the HCVR/LCVR amino acid sequence pair of SEQ ID NOs: ½ at a concentration of 150 mg/mL was formulated with 20 mM histidine, 12.5 mM sodium acetate, 25 mM arginine-HCl, 5% w/v sucrose, and polysorbate 20, polysorbate 80, PEG3350 or poloxamer 188 at varying concentrations, at pH 5.9. These formulations were subjected to thermal stress (45° C.)for a period of up to 56 days, and the percentage of high molecular weight (HMW) species was determined by size exclusion ultra high performance liquid chromatography (SE-UPLC) at 7 days, at 14 days, at 28 days, at 42 days, and at 56 days.

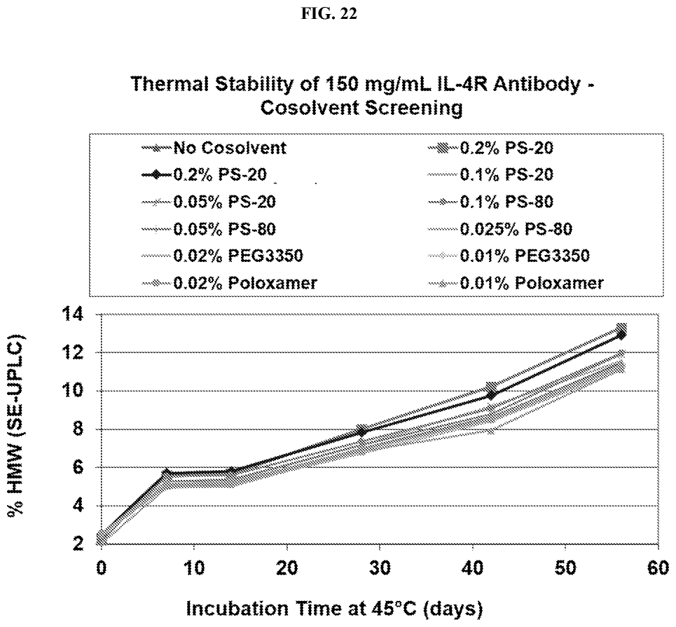

As shown in FIG. 22, antibody formulations comprising PEG3350 or poloxamer 188 at a concentration of 0.01% or 0.02% showed similar thermal stability to antibody formulations containing lower amounts of polysorbate (up to 0.1% w/v). By 28 days, the PEG3350—or poloxamer 188-containing formulations exhibited a lower percentage of HMW species than control formulations comprising 0.2% polysorbate 20.

Additional studies were carried out comparing the thermal stability of antibody formulations containing 0.2% polysorbate 80 (Table 18), 0.01% poloxamer 188 (Table 19), 0.1% poloxamer 188 (Table 20), 0.5% poloxamer 188 (Table 21), 0.01% PEG3350 (Table 22), 0.1% PEG3350 (Table 23), or 0.5% PEG3350 (Table 24). Each formulation comprised 150 mg/mL anti-IL4R antibody, 25 mM L-arginine-HCl, 20 mM L-histidine, 12.5 mM sodium acetate, 5% (w/v) sucrose, and the surfactant, at a pH of 5.9. Formulations comprising poloxamer 188 or PEG3350 showed stability benefits compared to a formulation comprising polysorbate 80.

TABLE 18
Thermal stability of formulations with 0.2% PS80

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.02 | 0.16 | 0.33 |
| pH | | 6.0 | 6.0 | 6.0 | 6.0 |
| % antibody Recovered by RP-UPLC | | 100 | 98 | 97 | 94 |
| Purity by SE-UPLC | % HMW | 2.1 | 10.3 | 18.6 | 26.6 |
| | % Main | 97.2 | 88.7 | 79.0 | 70.7 |
| | % LMW | 0.7 | 1.0 | 2.4 | 2.7 |
| | % Acidic | 27.7 | 53.7 | 77.9 | 86.9 |
| Charge Variant Analysis | % Main | 62.5 | 41.0 | 20.9 | 9.5 |
| by CEX-UPLC | % Basic | 9.9 | 5.3 | 1.3 | 3.6 |
| MFI (# particles/mL) | 2-10 μm | 125 | 1428 | 1785 | 377 |
| | ≥10 μm | 23 | 29 | 209 | 19 |
| | ≥25 μm | 4 | 2 | 23 | 4 |

TABLE 19
Thermal stability of formulations with 0.01% poloxamer 188

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.02 | 0.18 | 0.28 |
| pH | | 6.1 | 6.0 | 6.1 | 6.0 |
| % antibody Recovered by RP-UPLC | | 100 | 100 | 97 | 98 |
| Purity by SE-UPLC | % HMW | 2.1 | 9.1 | 16.3 | 23.7 |
| | % Main | 97.2 | 90.0 | 81.4 | 73.5 |
| | % LMW | 0.7 | 0.9 | 2.3 | 2.7 |
| Charge Variant Analysis | % Acidic | 28.4 | 51.7 | 75.5 | 86.6 |
| by CEX-UPLC | % Main | 62.1 | 43.1 | 21.0 | 8.9 |
| | % Basic | 9.5 | 5.3 | 3.6 | 4.5 |
| MFI (# particles/mL) | 2-10 μm | 79 | 142 | 648 | 396 |
| | ≥10 μm | 17 | 17 | 23 | 8 |
| | ≥25 μm | 0 | 6 | 2 | 0 |

TABLE 20
Thermal stability of formulations with 0.1% poloxamer 188

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.01 | 0.14 | 0.21 |
| pH | | 6.1 | 6.0 | 6.1 | 6.0 |
| % antibody Recovered by RP-UPLC | | 100 | 99 | 97 | 100 |
| Purity by SE-UPLC | % HMW | 2.1 | 9.1 | 16.4 | 23.7 |
| | % Main | 97.3 | 90.0 | 81.2 | 73.6 |
| | % LMW | 0.6 | 0.9 | 2.4 | 2.8 |
| Charge Variant Analysis | % Acidic | 28.1 | 51.9 | 74.9 | 84.0 |
| by CEX-UPLC | % Main | 62.1 | 46.2 | 21.0 | 8.7 |
| | % Basic | 9.8 | 1.9 | 4.1 | 7.4 |
| MFI (# particles/mL) | 2-10 μm | 69 | 540 | 993 | 811 |
| | ≥10 μm | 0 | 17 | 38 | 25 |
| | ≥25 μm | 0 | 0 | 2 | 0 |

TABLE 21
Thermal stability of formulations with 0.5% poloxamer 188

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.02 | 0.06 | 0.22 |
| pH | | 6.1 | 6.1 | 6.1 | 6.0 |
| % antibody Recovered by RP-UPLC | | 100 | 100 | 97 | 100 |
| Purity by SE-UPLC | % HMW | 2.1 | 9.1 | 16.8 | 24.2 |
| | % Main | 97.3 | 90.0 | 80.9 | 73.1 |
| | % LMW | 0.6 | 0.9 | 2.4 | 2.7 |
| Charge Variant Analysis | % Acidic | 29.5 | 50.8 | 77.1 | 86.6 |
| by CEX-UPLC | % Main | 61.5 | 44.5 | 21.5 | 9.0 |
| | % Basic | 9.1 | 4.8 | 1.4 | 4.4 |
| MFI (# particles/mL) | 2-10 μm | 179 | 805 | 4089 | 1887 |
| | ≥10 μm | 10 | 8 | 671 | 307 |
| | ≥25 μm | 0 | 2 | 94 | 61 |

TABLE 22
Thermal stability of formulations with 0.01% PEG3350

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.03 | 0.14 | 0.23 |
| pH | | 6.1 | 6.1 | 6.1 | 6.0 |
| % antibody Recovered by RP-UPLC | | 100 | 102 | 98 | 100 |
| Purity by SE-UPLC | % HMW | 2.1 | 9.3 | 16.5 | 23.6 |
| | % Main | 97.4 | 89.9 | 81.2 | 73.6 |
| | % LMW | 0.6 | 0.9 | 2.4 | 2.7 |
| Charge Variant Analysis | % Acidic | 29.4 | 51.6 | 77.3 | 86.4 |
| by CEX-UPLC | % Main | 62.2 | 46.1 | 21.5 | 11.9 |
| | % Basic | 8.5 | 2.4 | 1.1 | 1.8 |
| MFI (# particles/mL) | 2-10 μm | 42 | 494 | 1216 | 2085 |
| | ≥10 μm | 6 | 15 | 44 | 206 |
| | ≥25 μm | 0 | 6 | 6 | 40 |

TABLE 23
Thermal stability of formulations with 0.1% PEG3350

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.03 | 0.15 | 0.23 |
| pH | | 6.0 | 6.0 | 6.1 | 6.1 |
| % antibody Recovered by RP-UPLC | | 100 | 97 | 92 | 92 |
| Purity by SE-UPLC | % HMW | 2.1 | 9.3 | 16.1 | 22.4 |
| | % Main | 97.4 | 89.8 | 81.6 | 74.9 |
| | % LMW | 0.6 | 0.9 | 2.4 | 2.8 |
| Charge Variant Analysis | % Acidic | 32.1 | 51.0 | 77.6 | 85.7 |
| by CEX-UPLC | % Main | 60.6 | 45.6 | 21.5 | 10.9 |
| | % Basic | 7.4 | 3.4 | 0.9 | 3.5 |
| MFI (# particles/mL) | 2-10 μm | 94 | 313 | 1541 | 2058 |
| | ≥10 μm | 2 | 15 | 90 | 159 |
| | ≥25 μm | 0 | 4 | 0 | 21 |

TABLE 24
Thermal stability of formulations with 0.5% PEG3350

| | | | Length of Storage at 45° C. | | |
|---|---|---|---|---|---|
| | | | (months) | | |
| Assay | | t = 0 | 1 | 2 | 3 |
| Visual Appearance | | Pass | Pass | Pass | Pass |
| Increase in Turbidity (OD 405 nm) | | — | 0.02 | 0.15 | 0.23 |
| pH | | 6.1 | 6.1 | 6.1 | 6.0 |
| % antibody Recovered by RP-UPLC | | 100 | 100 | 97 | 98 |
| Purity by SE-UPLC | % HMW | 2.1 | 9.2 | 16.7 | 23.9 |
| | % Main | 97.3 | 89.8 | 81.0 | 73.4 |
| | % LMW | 0.6 | 0.9 | 2.4 | 2.7 |
| Charge Variant Analysis | % Acidic | 29.7 | 52.0 | 75.6 | 83.6 |
| by CEX-UPLC | % Main | 62.0 | 45.7 | 21.3 | 10.5 |
| | % Basic | 8.4 | 2.3 | 3.1 | 6.0 |
| MFI (# particles/mL) | 2-10 μm | 121 | 1289 | 528 | 2654 |
| | ≥10 μm | 0 | 44 | 42 | 279 |
| | ≥25 μm | 0 | 25 | 15 | 31 |

CEX, cation exchange; FCP, final concentrated pool; HMW, high molecular weight; LMW, low molecular weight; MFI, Micro-Flow Imaging; NR, not required; OD, optical density; RH, relative humidity; RP, Reverse Phase; SE, size exclusion; UPLC, ultra-performance liquid chromatography

### Example 9. Reduction of Lipase Activity Using Agitation Stress and Heat Stress

In order to further reduce esterase and lipase activity, free fatty acid particle formation and/or polysorbate degradation in therapeutic products, additional methods for reducing esterase and lipase activity in formulated drug substance were investigated. It was surprisingly discovered that subjecting drug substance to stress conditions, such as agitation stress or heat stress, could be used to reduce esterase and lipase activity and increase the long-term stability of therapeutic molecules and surfactants in drug substance and subsequent drug products.

mAb1 drug substance produced without a HIC step was subjected to agitation stress. 30 mL of 200 mg/mL mAb1 DS was used. Two 125 mL polycarbonate bottles were each filled with 10 mL of DS and agitated on an orbital shaker at 250 rpm for 0, 24, or 48 hours. The remaining 10 mL of DS were transferred to a 15 mL Falcon tube and served as the unstressed control. 2 mL of each DS was analyzed as either pre-stressed (control) DS (0 hours of agitation) or post-stressed DS (24 or 48 hours of agitation).

The control or stressed DS was used to prepare 150 mg/mL final drug substance (FDS), comprising 150 mg/mL mAb1, 20 mM histidine, 12.5 mM acetate, 5% sucrose, 0.2% high purity PS20, and 25 mM arginine HCl, at pH 5.9. The FDS was filter sterilized and then stored for 0 weeks, 4 weeks, or 8 weeks at 45° C. Storage at 45° C. was selected in order to accelerate the stability study. Physical stability of every sample collected above was analyzed using visual inspection for visible particles and aggregates, SE-UPLC for high and low molecular weight species, micro-flow imaging for subvisible particulate (2-300 μm) analysis, and CAD-UPLC for determining PS20 levels.

mAb1 drug substance subjected to the agitation stress and filtering steps showed less particle formation and greater polysorbate retention over time, as shown in Table 25.

TABLE 25
Thermal stability of formulations subjected to agitation stress

| | | Length of Agitation at 250 rpm (hours) | | | | | | | | |
|---|---|---|---|---|---|---|---|---|---|---|
| | | 0 | | | 24 | | | 48 | | |
| Length of Storage at 45° C. (weeks) | | 0 | 4 | 8 | 0 | 4 | 8 | 0 | 4 | 8 |
| Visual Appearance | | Pass | Pass | Pass | Pass | Pass | Pass | Pass | Pass | Pass |
| Polysorbate Quantification % by CAD | | 100 | 32 | 25 | 100 | 59 | 48 | 100 | 65 | 33 |
| Purity by SE-UPLC | % HMW | 1.7 | | | 16.6 | | | 30.9 | | |
| | % Main | 98.3 | | | 83.5 | | | 69.1 | | |
| | % LMW | 0 | | | 0 | | | 0 | | |
| MFI (# particles/mL) | 2-10 μm | 1036 | 3260 | 503066 | 739 | 3955 | 7792 | 1269 | 13974 | 14415 |
| | ≥10 μm | 54 | 53 | 81146 | 15 | 53 | 39 | 73 | 140 | 125 |
| | ≥25 μm | 10 | 6 | 17563 | 3 | 3 | 0 | 7 | 14 | 3 |

^{a} MFI USP specifications: ≥10 μm sizes ≤3000 particles/container; ≥25 μm sizes ≤300 particles/container
CAD, charged aerosol detector; CEX, cation exchange; FCP, final concentrated pool; HMW, high molecular weight; LMW, low molecular weight; MFI, Micro-Flow Imaging; NR, not required; OD, optical density; RH, relative humidity; RP, Reverse Phase; SE, size exclusion; UPLC, ultra-performance liquid chromatography.

Additional forms of stress were also investigated. mAb1 drug substance produced without a HIC step was subjected to heat stress. DS was stored at 45° C. for 0, 0.5, or 1 months in order to cause lipases to degrade, aggregate and/or inactivate. The control and stressed DS were used to prepare 150 mg/mL FDS, comprising 150 mg/mL mAb1, 20 mM histidine, 12.5 mM acetate, 5% sucrose, 0.2% high purity polysorbate 20, and 25 mM arginine HCl, at pH 5.9. The FDS was filter sterilized and then stored under conditions set forth in Table 26.

TABLE 26
Incubation/storage conditions for FDS from heat stressed DS

| Stress Condition | Length of Incubation |
|---|---|
| No storage | t = 0 |
| 45° C. | 0.5, 1, 1.5, 2 months |
| 25° C. | 3, 6 months |
| 5° C. | 3, 6, 12, 18, 24, 36 months & 3 backups |

Physical stability of the thermally stressed and control non-stressed samples described above was analyzed using visual inspection for visible particles and aggregates, SE-UPLC for high and low molecular weight species, micro-flow imaging for subvisible particulate (2-300 μm) analysis, and CAD-UPLC for determining PS20 levels. Results are set forth below in Tables 27-32. Subjecting DS to thermal stress prior to formulation resulted in a significant decrease of lipase activity, improving polysorbate recovery and decreasing free fatty acid particle formation. Non-stressed samples showed a significant increase in subvisible particles over time that was not observed in stressed samples. Subjecting DS to thermal stress did result in a significant increase in HMW level, but that level remained stable during further incubation.

TABLE 27
Stability of a formulation from DS with 0 months of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | |
| Increase in Turbidity (OD | | — | | | | | | |
| 405 nm) | | | | | | | | |
| pH | | | | | | | | |
| Polysorbate Quantification | | 100 | | 53 | | | | |
| % by CAD | | | | | | | | |
| Purity by SE- | % HMW | 2.2 | 2.2 | 2.5 | 2.5 | 2.6 | 2.7 | 2.8 |
| UPLC | % Main | 96.6 | 96.6 | 95 | 96.6 | 96.5 | 95.9 | 95.4 |
| | % LMW | 1.2 | 1.2 | 2.5 | 0.9 | 0.9 | 1.5 | 1.8 |
| MFI (# | 2-10 μm | 264 | 12265 | 81,203 | 141583 | 243895 | 273027 | 371554 |
| particles/mL) | ≥10 μm | 20 | 2273 | 8446 | 16902 | 26739 | 3042 | 38956 |
| | ≥25 μm | 3 | 9 | 47 | 530 | 1780 | 113 | 3602 |

TABLE 28
Stability of a formulation from DS with 0.5 months of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | |
| Increase in Turbidity (OD | | — | | | | | | |
| 405 nm) | | | | | | | | |
| pH | | | | | | | | |
| Polysorbate Quantification | | 100 | | 89 | | | | |
| % by CAD | | | | | | | | |
| Purity by SE- | % HMW | 10.9 | 10.7 | 11 | 12 | 12 | 11.6 | 11.7 |
| UPLC | % Main | 86.3 | 86.6 | 85.7 | 85.8 | 85.7 | 85.7 | 84.9 |
| | % LMW | 2.8 | 2.7 | 3.4 | 2.2 | 2.3 | 2.7 | 3.4 |
| MFI (# | 2-10 μm | 349 | 6691 | 5828 | 1794 | 3052 | 21842 | 258288 |
| particles/mL) | ≥10 μm | 16 | 216 | 61 | 34 | 222 | 3641 | 3586 |
| | ≥25 μm | 2 | 21 | 8 | 6 | 36 | 248 | 119 |

TABLE 29
Stability of a formulation from DS with 1 month of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | |
| Increase in Turbidity (OD | | — | | | | | | |
| 405 nm) | | | | | | | | |
| pH | | | | | | | | |
| Polysorbate Quantification | | 100 | | 90 | | | | |
| % by CAD | | | | | | | | |
| Purity by SE- | % HMW | 17.3 | 17.4 | 17.7 | 19 | 18.9 | 18.6 | 18.5 |
| UPLC | % Main | 78.5 | 78.4 | 77.6 | 77.6 | 77.6 | 77.2 | 76.7 |
| | % LMW | 4.2 | 4.2 | 4.7 | 3.5 | 3.5 | 4.2 | 4.8 |
| MFI (# | 2-10 μm | 160 | 3898 | 2081 | 35220 | 4806 | 824 | 5189 |
| particles/mL) | ≥10 μm | 7 | 52 | 14 | 1017 | 119 | 39 | 109 |
| | ≥25 μm | 2 | 6 | 1 | 30 | 5 | 2 | 8 |

TABLE 30
Stability of a formulation from DS with 0 months of thermal stress

| | | | Length of Storage at | | Length of Storage at | | | |
|---|---|---|---|---|---|---|---|---|
| | | | 25° C. (months) | | 45° C. (months) | | | |
| Assay | | t = 0 | 3 | 6 | 0.5 | 1 | 1.5 | 2 |
| Visual Appearance | | Pass | Pass | Pass | Pass | | Pass | |
| Increase in Turbidity (OD | | — | | | | | | |
| 405 nm) | | | | | | | | |
| pH | | | | | | | | |
| PS-20 level (CAD-UPLC) | | 100 | | 16 | | | | 33 |
| Purity by SE- | % HMW | 2.2 | 3 | 3.5 | 6.2 | 8.5 | 13.3 | 17.4 |
| UPLC | % Main | 96.6 | 95.8 | 94.2 | 92.2 | 89.2 | 84.4 | 79.9 |
| | % LMW | 1.2 | 1.2 | 2.3 | 1.6 | 2.3 | 2.3 | 2.7 |
| MFI (# | 2-10 μm | 264 | 19609 | 146335 | 25146 | 2532 | 8226 | 263410 |
| particles/mL) | ≥10 μm | 20 | 492 | 23326 | 750 | 47 | 206 | 41033 |
| | ≥25 μm | 3 | 19 | 3825 | 60 | 5 | 20 | 9790 |

TABLE 31
Stability of a formulation from DS with 0.5 months of thermal stress

| | | | Length of Storage at | | Length of Storage at | | | |
|---|---|---|---|---|---|---|---|---|
| | | | 25° C. (months) | | 45° C. (months) | | | |
| Assay | | t = 0 | 3 | 6 | 0.5 | 1 | 1.5 | 2 |
| Visual Appearance | | Pass | Pass | Pass | Pass | | Pass | |
| Increase in Turbidity (OD | | — | | | | | | |
| 405 nm) | | | | | | | | |
| pH | | | | | | | | |
| PS-20 level (CAD-UPLC) | | 100 | | 68 | | | | 75 |
| Purity by SE- | % HMW | 10.9 | 11 | 11.5 | 14.5 | 14.6 | 21.2 | 24.8 |
| UPLC | % Main | 86.3 | 86.2 | 84.4 | 82.3 | 81 | 74.5 | 70.3 |
| | % LMW | 2.8 | 2.8 | 4.2 | 3.2 | 4.4 | 4.4 | 4.9 |
| MFI (# | 2-10 μm | 349 | 6232 | 8660 | 4838 | 9249 | 18683 | 7797 |
| particles/mL) | ≥10 μm | 16 | 52 | 169 | 60 | 172 | 426 | 272 |
| | ≥25 μm | 2 | 4 | 3 | 8 | 0 | 29 | 6 |

TABLE 32
Stability of a formulation from DS with 1 month of thermal stress

| | | | Length of Storage at | | Length of Storage at | | | |
|---|---|---|---|---|---|---|---|---|
| | | | 25° C. (months) | | 45° C. (months) | | | |
| Assay | | t = 0 | 3 | 6 | 0.5 | 1 | 1.5 | 2 |
| Visual Appearance | | Pass | Pass | Pass | Pass | | Pass | |
| Increase in Turbidity (OD | | — | | | | | | |
| 405 nm) | | | | | | | | |
| pH | | | | | | | | |
| PS-20 level (CAD-UPLC) | | 100 | | 77 | | | | 84 |
| Purity by SE- | % HMW | 17.3 | 17.5 | 17.6 | 20.8 | 19.4 | 27 | 30.2 |
| UPLC | % Main | 78.5 | 78.4 | 77.2 | 74.6 | 74.4 | 67.4 | 63.6 |
| | % LMW | 4.2 | 4.2 | 5.2 | 4.6 | 6.2 | 5.6 | 6.3 |
| MFI (# | 2-10 μm | 160 | 5799 | 11893 | 3258 | 10647 | 18440 | 4921 |
| particles/mL) | ≥10 μm | 7 | 84 | 236 | 38 | 359 | 453 | 70 |
| | ≥25 μm | 2 | 3 | 19 | 10 | 18 | 28 | 4 |

HCPs are expected to disproportionately inactivate, degrade and aggregate in response to stress due to a reduced stability compared to a biotherapeutic, for example a therapeutic antibody. HMW species in DS subjected to agitation stress or thermal stress, whether formed from HCPs, drug protein, or a combination, can be removed using further processing steps such as filtration or chromatography. Thermally stressed DS was subjected to cation exchange (CEX) chromatography to remove HMW species that formed during stress conditions. HMW species were efficiently depleted from stressed DS, as shown in Table 33.

TABLE 33
Clearance of HMW species from stressed drug substance

| | | | Thermal stress of 1 | | Thermal stress of 2 | |
|---|---|---|---|---|---|---|
| | | | week | | weeks | |
| Assay | | t = 0 | 45° C. | 50° C. | 45° C. | 50° C. |
| Purity by SE-UPLC before | % HMW | 1.7 | 10.2 | 26.2 | 17.0 | 33.6 |
| CEX purification | % Main | 98.3 | 89.8 | 73.8 | 83.0 | 66.4 |
| Purity by SE-UPLC after | % HMW | 1.3 | 2.3 | 2.3 | 2.5 | 2.5 |
| CEX purification | % Main | 98.7 | 97.7 | 97.7 | 97.5 | 97.5 |

HMW-depleted DS was formulated into FDS comprising 150 mg/mL mAb1, 20 mM histidine, 12.5 mM acetate, 5% sucrose, 0.2% PS80, and 25 mM arginine HCl, at pH 5.9. The stability of FDS from HMW-depleted thermally stressed DS was compared to FDS from HMW-depleted non-stressed DS, as shown in Table 34. After only one week (0.25 months), FDS from HMW-depleted non-stressed DS showed greatly improved PS80 recovery compared to HMW-depleted non-stressed DS. Further improvements may be measured at later time points; the measurable formation of free fatty acid particles from accumulated free fatty acids is expected to follow in time after PS80 degradation in HMW-depleted non-stressed DS. It should be noted that a difference in lipase activity between stressed and non-stressed DS after HMW depletion may also be masked due to HCP lipase depletion from the non-stressed DS as a result of the chromatography step. These results demonstrate that the disclosed methods of subjecting drug substance to agitation stress or heat stress, optionally followed by HMW depletion, produce an improved pharmaceutical composition with reduced lipase activity, reduced polysorbate degradation, and reduced free fatty acid particle formation.

TABLE 34
Thermal stability of a formulation from HMW-depleted thermally stressed DS

| | | Drug Substance Thermal Stress | | | | | |
|---|---|---|---|---|---|---|---|
| | | Control | | 1 week at 45° C. | | 1 week at 50° C. | |
| Length of Storage at 45° C. (months) | | 0 | 0.25 | 0 | 0.25 | 0 | 0.25 |
| Visual Appearance | | Pass | Pass | Pass | Pass | Pass | Pass |
| Increase in Turbidity (OD 405 nm) | | 0.0 | 0.03 | 0.0 | 0.01 | 0.0 | 0.0 |
| pH | | 6.2 | 6.2 | 6.2 | 6.2 | 6.1 | 6.0 |
| PS-80 level (CAD-UPLC) | | 100 | 68 | 100 | 97 | 100 | 101 |
| Purity by SE-UPLC | % HMW | 2.2 | 4.0 | 2.7 | 3.8 | 3.0 | 4.0 |
| | % Main | 97.7 | 95.7 | 97.0 | 95.9 | 96.7 | 95.7 |
| | % LMW | 0.2 | 0.3 | 0.3 | 0.3 | 0.3 | 0.3 |
| % mAb1 recovered by RP-UPLC | | 100 | 103 | 100 | 103 | 100 | 103 |
| MFI (# particles/mL) | 2-10 μm | 876 | 438 | 323 | 646 | 469 | 947 |
| | ≥10 μm | 48 | 31 | 6 | 27 | 29 | 148 |
| | ≥25 μm | 2 | 4 | 0 | 2 | 8 | 48 |

The present invention is not to be limited in scope by the specific aspects described herein. Indeed, various modifications of the invention in addition to those described herein will become apparent to those skilled in the art from the foregoing description. Such modifications are intended to fall within the scope of the appended claims.

### Enumerated Examples

The following Enumerated Example set forth below provide additional aspects of the present disclosure.
1. A method for producing a pharmaceutical composition with reduced lipase, comprising: subjecting a sample including a protein of interest and a lipase to anion exchange chromatography, wherein a pH of the sample loaded to the chromatography column is between about 7.8 and about 8.3.
2. A method for producing a pharmaceutical composition with reduced lipase activity, comprising:
   (a) subjecting a sample including a protein of interest and a lipase to stress conditions to form a sample with inactivated lipase; and
   (b) formulating said sample with inactivated lipase to produce a pharmaceutical composition with reduced lipase activity.
3. The method of example 2, wherein said protein of interest is an antibody, an antibody-derived protein, an antibody fragment, a monoclonal antibody, a bispecific antibody, a fusion protein, an antibody-drug conjugate, or a therapeutic protein.
4. The method of example 2 or 3, wherein said formulating step comprises adding a fatty acid ester to said sample, wherein said fatty acid ester is polysorbate 20, polysorbate 40, polysorbate 60, or polysorbate 80.
5. The method of example 4, wherein said fatty acid ester is polysorbate 80 and a concentration of oleic acid esters in said polysorbate 80 is at least 80%.
6. The method of example 5, wherein a concentration of oleic acid esters in said polysorbate 80 is at least 98% or at least 99%.
7. The method of any one of examples 2-6, wherein said stress conditions include agitation stress and/or heat stress.
8. The method of example 7, wherein said agitation stress comprises shaking said sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm.
9. The method of example 7, wherein said agitation stress comprises shaking said sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.
10. The method of example 7, wherein said heat stress comprises storing said sample at from about 30° C. to about 60° C., from about 35° C. to about 55° C., from about 40° C. to about 50 ºC, from about 44° C. to about 46° C., about 30° C., about 35° C., about 40° ° C., about 45° C., about 50° C., about 55° C., or about 60° C.
11. The method of example 10, wherein said storage is from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.
12. The method of any one of example 2-11, further comprising subjecting said sample with inactivated lipase to filtration, enrichment, or chromatographic separation to remove high molecular weight (HMW) species prior to step (b).
13. The method of example 12, wherein said chromatographic separation comprises cation exchange chromatography.
14. The method of example 12, wherein said chromatographic separation comprises size exclusion chromatography.
15. The method of any one of examples 2-14, wherein said formulating step comprises adding excipients to said sample.
16. A method for producing a pharmaceutical composition with reduced lipase activity:
   (a) subjecting harvested antibody to affinity chromatography;
   (b) subjecting said antibody pooled from eluate of step (a) to viral inactivation at a pH from about 3 to about 4 and then adjusting the pH to from about 5 to about 8;
   (c) subjecting said antibody pooled from step (b) to anion exchange chromatography in flowthrough mode;
   (d) subjecting said antibody pooled from eluate of step (c) to hydrophobic interaction chromatography in flowthrough mode;
   (e) subjecting said antibody pooled from flowthrough fractions of step (d) to virus retentive filtration;
   (f) subjecting a sample from step (e) including an antibody of interest and a lipase to agitation stress or heat stress to form a pharmaceutical composition with reduced lipase activity.
17. A method for producing a pharmaceutical composition with reduced lipase activity:
   (a) subjecting harvested antibody to affinity chromatography;
   (b) subjecting said antibody pooled from eluate of step (a) to viral inactivation at a pH from about 3 to about 4 and then adjusting the pH to from about 5 to about 8;
   (c) subjecting said antibody pooled from step (b) to anion exchange chromatography in flowthrough mode;
   (d) subjecting said antibody pooled from flowthrough fractions of step (c) to virus retentive filtration;
   (e) subjecting a sample from step (d) including an antibody of interest and a lipase to agitation stress or heat stress to form a pharmaceutical composition with reduced lipase activity.
18. The method of example 16 or 17, further comprising subjecting said pharmaceutical composition to filtration, enrichment, or chromatographic separation to remove HMW species.
19. The method of example 18, wherein said chromatographic separation comprises ion exchange chromatography, cation exchange chromatography, or size exclusion chromatography.
20. The method of any one of claims 16-19, wherein a pH of the sample loaded to the AEX column is between about 7.8 and about 8.3.
21. A method for producing a pharmaceutical composition with increased stability, comprising: reducing lipase activity in a composition by subjecting a sample including a protein of interest and a lipase to anion exchange (AEX) chromatography, wherein a pH of the sample loaded to the AEX column is between about 7.8 and about 8.3.
22. The method of example 21, further comprising the step of subjecting a sample formed after AEX chromatography to agitation stress or heat stress to reduce lipase activity.
23. The method of any one of claims 16-22, further comprising the addition of poloxamer 188 or PEG3350 to produce a formulation comprising a pharmaceutical composition with reduced lipase activity and increased stability.
24. The method of example 23, wherein the formulation is substantially free of polysorbate.
25. The method of example 23 or 24, wherein a concentration of said poloxamer 188 is from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%.
26. The method of cl example aim 23 or 24, wherein a concentration of said PEG3350 is from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%.
27. A method for producing a pharmaceutical composition with increased stability, comprising: (a) subjecting a harvested recombinant protein to anion exchange chromatography in flowthrough mode;
   (b) subjecting flowthrough fractions from step (a) to hydrophobic interaction chromatography in flowthrough mode; and
   (c) formulating recombinant protein isolated from step (b) with polysorbate 80, wherein a concentration of oleic acid esters in said polysorbate 80 is at least 80%.
28. The method of example 27, wherein a concentration of said oleic acid esters at least 98% or at least 99%.
29. The method of any one of examples 1 or 16-28, wherein a pH of the sample loaded to the anion exchange chromatography column is from about 7.8 to about 8.3, from about 7.9 to about 8.2, about 7.8, about 7.9, about 8.0, about 8.1, about 8.2, or about 8.3.

What is claimed is:

1. A method for producing a pharmaceutical composition with reduced lipase activity, comprising:
(a) subjecting a sample including a protein of interest and a lipase to stress conditions to form a sample with inactivated lipase; and
(b) formulating said sample with inactivated lipase to produce a pharmaceutical composition with reduced lipase activity.

2. The method of claim 1, wherein said protein of interest is an antibody, an antibody-derived protein, an antibody fragment, a monoclonal antibody, a bispecific antibody, a fusion protein, an antibody-drug conjugate, or a therapeutic protein.

3. The method of claim 1, wherein said formulating step comprises adding a fatty acid ester to said sample, wherein said fatty acid ester is polysorbate 20, polysorbate 40, polysorbate 60, or polysorbate 80.

4. The method of claim 3, wherein said fatty acid ester is polysorbate 80 and a concentration of oleic acid esters in said polysorbate 80 is at least 80%.

5. The method of claim 4, wherein a concentration of oleic acid esters in said polysorbate 80 is at least 98% or at least 99%.

6. The method of claim 1, wherein said stress conditions include agitation stress and/or heat stress.

7. The method of claim 6, wherein said agitation stress comprises shaking said sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm.

8. The method of claim 6, wherein said agitation stress comprises shaking said sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.

9. The method of claim 6, wherein said heat stress comprises storing said sample at from about 30° ° C. to about 60° C., from about 35° ° C. to about 55° C., from about 40° ° C. to about 50° C., from about 44° C. to about 46° ° C., about 30° C., about 35° C., about 40° C., about 45° C., about 50° C., about 55° C., or about 60° C.

10. The method of claim 9, wherein said storage is from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.

11. The method of claim 1, further comprising subjecting said sample with inactivated lipase to filtration, enrichment, or chromatographic separation to remove high molecular weight (HMW) species prior to step (b).

12. The method of claim 11, wherein said chromatographic separation comprises cation exchange chromatography.

13. The method of claim 11, wherein said chromatographic separation comprises size exclusion chromatography.

14. The method of claim 1, wherein said formulating step comprises adding excipients to said sample.

15. The method of claim 1, wherein said sample is a drug substance.

16. A method for producing a pharmaceutical composition with reduced lipase activity, comprising:
(a) subjecting a drug substance including a protein of interest and a lipase to heat stress to form a drug substance with inactivated lipase;
(b) subjecting said drug substance with inactivated lipase to cation exchange chromatography to form a HMW-depleted drug substance; and
(c) formulating said HMW-depleted drug substance to produce a pharmaceutical composition with reduced lipase activity.

17. The method of claim 16, wherein said protein of interest is a monoclonal antibody.

18. The method of claim 16, wherein said formulating step comprises adding polysorbate 20 or polysorbate 80.

19. The method of claim 16, wherein said heat stress comprises storing said sample at from about 45° ° C. to about 50° C.

20. The method of claim 19, wherein said storage is from about 0.5 months to about 2 months.


SEQUENCE LISTING (188 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 16 aa, synthetic construct
AKDRLSITIRPRYYGL

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 46 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 52 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 53 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 58 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 64 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 65 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 66 - protein, 5 aa, synthetic construct
RNAMF

SEQ ID NO: 67 - protein, 16 aa, synthetic construct
GIGTGGATSYADSVKG

SEQ ID NO: 68 - protein, 7 aa, synthetic construct
GRYYFDY

SEQ ID NO: 69 - protein, 12 aa, synthetic construct
RASQSVSSSYLA

SEQ ID NO: 70 - protein, 7 aa, synthetic construct
GASSRAT

SEQ ID NO: 71 - protein, 10 aa, synthetic construct
QQYDHSAGWT

SEQ ID NO: 72 - protein, 7 aa, synthetic construct
GASSRAP

SEQ ID NO: 73 - protein, 10 aa, synthetic construct
QQYGSSPPWT

SEQ ID NO: 74 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 79 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 80 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 81 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 82 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 83 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 86 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 87 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 89 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 90 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 91 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 93 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 95 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 96 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 97 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 98 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 99 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 101 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 103 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 104 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 105 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 106 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 109 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 110 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 111 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 112 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 113 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 114 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 115 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 116 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 117 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 118 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 119 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 120 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 121 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 122 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 123 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 124 - protein, 5 aa, synthetic construct
SYYMH

SEQ ID NO: 125 - protein, 17 aa, synthetic construct
IINPRGGSTSYAQKFQG

SEQ ID NO: 126 - protein, 7 aa, synthetic construct
GKYWMYD

SEQ ID NO: 127 - protein, 13 aa, synthetic construct
SGGGSSIGNSYVS

SEQ ID NO: 128 - protein, 7 aa, synthetic construct
DNNKRPS

SEQ ID NO: 129 - protein, 12 aa, synthetic construct
GTWDTSPVWEWP

SEQ ID NO: 130 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 131 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 132 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 133 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 134 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 135 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 136 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 137 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 138 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 139 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 140 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 141 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 142 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 143 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 144 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 145 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 146 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 147 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 148 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 149 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 150 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 151 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 152 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 153 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 154 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 155 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 156 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 157 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 158 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 159 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 160 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 161 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 162 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 163 - protein, 5 aa, synthetic construct
RHAMA

SEQ ID NO: 164 - protein, 17 aa, synthetic construct
AITSSGRSIYYADSVKG

SEQ ID NO: 165 - protein, 7 aa, synthetic construct
VHRAFDY

SEQ ID NO: 166 - protein, 13 aa, synthetic construct
SGSPLFPDSGSFN

SEQ ID NO: 167 - protein, 7 aa, synthetic construct
ADSHRPS

SEQ ID NO: 168 - protein, 11 aa, synthetic construct
GTWDYSLSGYV

SEQ ID NO: 169 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 170 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 171 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 172 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 173 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 174 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 175 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 176 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 177 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 178 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 179 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 180 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 181 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 182 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 183 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 184 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 185 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 186 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 187 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 188 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK